SEQUENCE LISTING (37 sequences)

SEQ ID NO: 1 - DNA, 3716 bp, homo sapiens
  CDS (101)..(1468)
cccgaggagccgggccccggccgctgtccagccgctccgtgccccgcgcgtcctgcgccgccgccaccgcctcctggggagacgcagccacttgcccgccatggatactcccagggtcctgctctcggccgtcttcctcatcagttttctgtgggatttgcccggtttccagcaggcttccatctcatcctcctcgtcgtccgccgagctgggttccaccaagggcatgcgaagccgcaaggaaggcaagatgcagcgggcgccgcgcgacagtgacgcgggccgggagggccaggaaccacagccgcggcctcaggacgaaccccgggctcagcagccccgggcgcaggagccgccaggcaggggtccgcgcgtggtgccccacgagtacatgctgtcaatctacaggacttactccatcgctgagaagctgggcatcaatgccagctttttccagtcttccaagtcggctaatacgatcaccagctttgtagacaggggactagacgatctctcgcacactcctctccggagacagaagtatttgtttgatgtgtccatgctctcagacaaagaagagctggtgggcgcggagctgcggctctttcgccaggcgccctcagcgccctgggggccaccagccgggccgctccacgtgcagctcttcccttgcctttcgcccctactgctggacgcgcggaccctggacccgcagggggcgccgccggccggctgggaagtcttcgacgtgtggcagggcctgcgccaccagccctggaagcagctgtgcttggagctgcgggccgcatggggcgagctggacgccggggaggccgaggcgcgcgcgcggggaccccagcaaccgccgcccccggacctgcggagtctgggcttcggccggagggtgcggcctccccaggagcgggccctgctggtggtattcaccagatcccagcgcaagaacctgttcgcagagatgcgcgagcagctgggctcggccgaggctgcgggcccgggcgcgggcgccgaggggtcgtggccgccgccgtcgggcgccccggatgccaggccttggctgccctcgcccggccgccggcggcggcgcacggccttcgccagtcgccatggcaagcggcacggcaagaagtccaggctacgctgcagcaagaagcccctgcacgtgaacttcaaggagctgggctgggacgactggattatcgcgcccctggagtacgaggcctatcactgcgagggtgtatgcgacttcccgctgcgctcgcacctggagcccaccaaccacgccatcatccagacgctgatgaactccatggaccccggctccaccccgcccagctgctgcgtgcccaccaaattgactcccatcagcattctatacatcgacgcgggcaataatgtggtctacaagcagtacgaggacatggtggtggagtcgtgcggctgcaggtagcggtgcctttcccgccgccttggcccggaaccaaggtgggccaaggtccgccttgcaggggaggcctggctgcagagaggcggaggaggaagctggcgctgggggaggctgagggtgagggaacagcctggatgtgagagccggtgggagagaagggagcgcagccttcccagtaacttctacctgccagcccagagggaaatatggattttcacaccttgcctggccaccctggaaaaacaagccaaggaggatttcttttgttctgttttctctctctctctctctctctctctctctctctctctctctctctctattactgtggctttggatttccttatgtgtcttacaggctttgatagaaggggaggggaggagagatgcatacccgtttctcaactgctccatggattgaaaaaataacagtttaaaaagggaaacaatgtgggaggaagaatcaccgttgacgcatcttgatttggttggtttttacatgtgtaaagaaggtggggtctctggccatgtcatagcccatgtcttgtgccctcccacacagaaagtgttagatagggaaattggcaaaaagaatagttaagtcaggaatggtcctgcctatagaagagctttgagagaggtgggcccacgggtgcccctctcacccatttgtgtactctgtgagtttaccagctctgccctggcctctttcggtaccaggaactggcaaccttcatctcactcctgagggcccaggtctctgccttcattgttgctttttctggtgggggcaaggggagctggtatggatggaatgacaagaattagtccaaatggaaccccttgaaggataatgagaaaccacaaggcctgcctctgactggggctgacacggaggtgcattagcccaggctggaggtagcccacccaaatgccctttctgattctaattgatttctttcaacagaatttgccaaaattcagacatgcacttctaaggggaaggtgattttccagttcaaaaaaatgggcaggagtggggaacaaaacaattaacgtaagagctacaaaggagggaaaaggaaccaagaagtagaaggagtcccatcaggagggaagatggtgggcctcagggaggatggggatcaagggacaggccaggagccaggagtggggaagggagggatgaaaggggacacaagtccctgtctctgaagtttctttaaaatctgagttccctcccctctctttgacattcctgaaagattaccagccagcaatagcccagggctcccccaaaagaattggttcagattgtaattatcagttaggcaatgtttttaaaacttagtaatgagaaactgtgaaaagagccaagtgttacattgagcttggggtgggagatggggaacaggcagtgaggaaggagacaggggtggaattcgtcttctgggaggaagctggagagagcacagtgaaattgaaatacccattcccagatagtcaaaaacatgaactttcccccagcctgcaccagtattgttttcaaacattgcccatgagtaggccctttgaagagttagcttcctcctcatctttgactataaaattgtttaatcaatggaatttgtaccagccttttaaaaagttttagtttttcctaagtgattttgctctcttccaatctaaacctgttgcttgtttggttcagagaactacaaactgtcaaagaaagggtggggatgataagaaatgctaatataaaaatgctaagtgaaaaaaagacttggccaggagaaataatttaaaatgcacatttgctttggatgcactgttgttctgttaaggctgtatatatttgtttatttaaggtgactgaaagtgcaaagaggaaatggacagcatgcaattcatcctaatgtacaaaacgttatatgcactcaaatgttataatttctaatatttttaaagtttatattcgagttgtacaaagttaagcattaatcagatatttcattttttcataatgttaccattttcttaaatattattacaaaattttaagtctgtctaatggagagttttttttaaactgtctacctcatataatacaagtatttacaacgctaaagttaccagaggtcaatgaataatcaaaacattttttacagtacacctttcctggatgatatgcaatcgaatgctatattattaaacgcatttttctccttattaaaaaaaaaaaaaaaaa

SEQ ID NO: 2 - protein, 455 aa, homo sapiens
MDTPRVLLSAVFLISFLWDLPGFQQASISSSSSSAELGSTKGMRSRKEGKMQRAPRDSDAGREGQEPQPRPQDEPRAQQPRAQEPPGRGPRVVPHEYMLSIYRTYSIAEKLGINASFFQSSKSANTITSFVDRGLDDLSHTPLRRQKYLFDVSMLSDKEELVGAELRLFRQAPSAPWGPPAGPLHVQLFPCLSPLLLDARTLDPQGAPPAGWEVFDVWQGLRHQPWKQLCLELRAAWGELDAGEAEARARGPQQPPPPDLRSLGFGRRVRPPQERALLVVFTRSQRKNLFAEMREQLGSAEAAGPGAGAEGSWPPPSGAPDARPWLPSPGRRRRRTAFASRHGKRHGKKSRLRCSKKPLHVNFKELGWDDWIIAPLEYEAYHCEGVCDFPLRSHLEPTNHAIIQTLMNSMDPGSTPPSCCVPTKLTPISILYIDAGNNVVYKQYEDMVVESCGCR

SEQ ID NO: 3 - protein, 174 aa, Homo sapiens
TRSQRKNLFAEMREQLGSAEAAGPGAGAEGSWPPPSGAPDARPWLPSPGRRRRRTAFASRHGKRHGKKSRLRCSKKPLHVNFKELGWDDWIIAPLEYEAYHCEGVCDFPLRSHLEPTNHAIIQTLMNSMDPGSTPPSCCVPTKLTPISILYIDAGNNVVYKQYEDMVVESCGCR

SEQ ID NO: 4 - DNA, 1940 bp, Homo sapiens
  CDS (236)..(1147)
agggcccggagccggcgagtgctcccgggaactctgcctgcgcggcggcagcgaccggaggccaggcccagcacgccggagctggcctgctggggaggggcgggaggcgcgcgcgggagggtccgcccggccagggccccgggcgctcgcagaggccggccgcgctcccagcccgcccggagcccatgcccggcggctggccagtgctgcggcagaagggggggcccggctctgcatggccccggctgctgacatgacttctttgccactcggtgtcaaagtggaggactccgccttcggcaagccggcggggggaggcgcgggccaggcccccagcgccgccgcggccacggcagccgccatgggcgcggacgaggagggggccaagcccaaagtgtccccttcgctcctgcccttcagcgtggaggcgctcatggccgaccacaggaagccgggggccaaggagagcgccctggcgccctccgagggcgtgcaggcggcgggtggctcggcgcagccactgggcgtcccgccggggtcgctgggagccccggacgcgccctcttcgccgcggccgctcggccatttctcggtggggggactcctcaagctgccagaagatgcgctcgtcaaagccgagagccccgagaagcccgagaggaccccgtggatgcagagcccccgcttctccccgccgccggccaggcggctgagccccccagcctgcaccctccgcaaacacaagacgaaccgtaagccgcggacgcccttcaccaccgcgcagctgctggcgctggagcgcaagttccgccagaagcagtacctgtccatcgccgagcgcgcggagttctccagctcgctcagcctcactgagacgcaggtgaagatatggttccagaaccgccgcgccaaggcaaagagactacaagaggcagagctggagaagctgaagatggccgccaagcccatgctgccaccggctgccttcggcctctccttccctctcggcggccccgcagctgtagcggccgcggcgggtgcctcgctctacggtgcctctggccccttccagcgcgccgcgctgcctgtggcgcccgtgggactctacacggcccatgtgggctacagcatgtaccacctgacatagagggtcccaggtcgcccacctgtgggccagccgattcctccagccctggtgctgtacccccgacgtgctcccctgctcggcaccgccagccgccttccctttaaccctcacactgctccagtttcacctctttgctccctgagttcactctccgaagtctgatccctgccaaaaagtggctggaagagtcccttagtactcttctagcatttagatctacactctcgagttaaagatggggaaactgagggcagagaggttaacagatttatctaaggtccccagcagaattgacagttgaacagagctagaggccatgtctcctgcatagcttttccctgtcctgacaccaggcaagaaaagcgcagagaaatcggtgtctgacgattttggaaatgagaacaatctcaaaaaaaaaaaaaaaaaaaaaaaaaaaaaaaaaaaagaaaagagaaaaaaaagactagccagccaggaagatgaatcctagcttcttccattggaaaatttaagacaagttcaacaacaaaacatttgctctggggggcagggaaaacacagatgtgttgcaaaggtaggttgaagggacctctctcttaccagtaccagaaacacaattgtaaaattaaaaaaaaaaaaaaactctttctatttaacagtacatttgtgtggctctcaaacatccctttggaagggattgtgtgtactatgtaatatactgtatatttgaaattttattatcatttatattatagctatatttgttaaataaattaattttaagctacaaaaa

SEQ ID NO: 5 - protein, 303 aa, Homo sapiens
MAPAADMTSLPLGVKVEDSAFGKPAGGGAGQAPSAAAATAAAMGADEEGAKPKVSPSLLPFSVEALMADHRKPGAKESALAPSEGVQAAGGSAQPLGVPPGSLGAPDAPSSPRPLGHFSVGGLLKLPEDALVKAESPEKPERTPWMQSPRFSPPPARRLSPPACTLRKHKTNRKPRTPFTTAQLLALERKFRQKQYLSIAERAEFSSSLSLTETQVKIWFQNRRAKAKRLQEAELEKLKMAAKPMLPPAAFGLSFPLGGPAAVAAAAGASLYGASGPFQRAALPVAPVGLYTAHVGYSMYHLT

SEQ ID NO: 6 - DNA, 2224 bp, Homo sapiens
  CDS (89)..(892)
tcccgtctccgcagcaaaaaagtttgagtcgccgctgccgggttgccagcggagtcgcgcgtcgggagctacgtagggcagagaagtcatggcttctccgtccaaaggcaatgacttgttttcgcccgacgaggagggcccagcagtggtggccggaccaggcccggggcctgggggcgccgagggggccgcggaggagcgccgcgtcaaggtctccagcctgcccttcagcgtggaggcgctcatgtccgacaagaagccgcccaaggaggcgtccccgctgccggccgaaagcgcctcggccggggccaccctgcggccactgctgctgtcggggcacggcgctcgggaagcgcacagccccgggccgctggtgaagcccttcgagaccgcctcggtcaagtcggaaaattcagaagatggagcggcgtggatgcaggaacccggccgatattcgccgccgccaagacatatgagccctaccacctgcaccctgaggaaacacaagaccaatcggaagccgcgcacgccctttaccacatcccagctcctcgccctggagcgcaagttccgtcagaaacagtacctctccattgcagagcgtgcagagttctccagctctctgaacctcacagagacccaggtcaaaatctggttccagaaccgaagggccaaggcgaaaagactgcaggaggcagaactggaaaagctgaaaatggctgcaaaacctatgctgccctccagcttcagtctccctttccccatcagctcgcccctgcaggcagcgtccatatatggagcatcctacccgttccatagacctgtgcttcccatcccgcctgtgggactctatgccacgccagtgggatatggcatgtaccacctgtcctaaggaagaccagatcaatagactccatgatggatgcttgtttcaaagggtttcctctccctctccacgaaggcagtaccagccagtactcctgctctgctaaccctgcgtgcaccaccctaagcggctaggctgacagggccacacgacatagctgaaatttgttctgtaggcggaggcaccaagccctgttttcttggtgtaatcttccagatgcccccttttcctttcacaaagattggctctgatggtttttatgtataaatatatatatataataaaatataatacatttttatacagcagacgtaaaaattcaaattattttaaaaggcaaaatttatatacatatgtgctttttttctatatctcaccttcccaaaagacactgtgtaagtccatttgttgtattttcttaaagagggagacaaattatttgcaaaatgtgctaaagtcaatgatttttacgggattattgacttctgcttatggaaaacaaagaaacagacacaatgcacacagaaaatattagatatggagagattattcaaagtgaaggggacacatcatatttctgcattttacttgcattaaaagaaacctctttatatactacagttgttcctatctctcccccgccccccaccgccccaccacacacatatttttaaagtttttccttttttaagaatatttttgtaagaccaatacctgggatgagaagaatcctgagactgcctggaggtgaggtagaaaattagaaatacttcctaattcttctcaaggctgttggtaactttatttcagataattggagagtaaaatgttaaaacctgttgagaggaattgatggtttctgagaaatactaggtacattcatcctcacagattgcaaaggtgatttgggtgggggtttagtaattttctgcttaaaaaatgagtatcttgtaaccattacctatatgctaaatattcttgaacaattagtagatccagaaagaaaaaaaaatatgctttctctgtgtgtgtacctgttgtatgtcctaaacttattagaaaattttatatacttttttacatgttggggggcagaaggtaaagccatgttttgacttggtgaaaatgggattgtcaaacagcccattaagttccctggtatttcaccttcctgtccatctgtcccctccctccggtatacctttatccctttgaaagggtgcttgtacaatttgatatattttattgaagagttatctcttattctgaattaaattaagcatttgttttattgcagtaaagtttgtccaaactcacaattaaaaaaaaaaaaaaaaa

SEQ ID NO: 7 - protein, 267 aa, Homo sapiens
MASPSKGNDLFSPDEEGPAVVAGPGPGPGGAEGAAEERRVKVSSLPFSVEALMSDKKPPKEASPLPAESASAGATLRPLLLSGHGAREAHSPGPLVKPFETASVKSENSEDGAAWMQEPGRYSPPPRHMSPTTCTLRKHKTNRKPRTPFTTSQLLALERKFRQKQYLSIAERAEFSSSLNLTETQVKIWFQNRRAKAKRLQEAELEKLKMAAKPMLPSSFSLPFPISSPLQAASIYGASYPFHRPVLPIPPVGLYATPVGYGMYHLS

SEQ ID NO: 8 - protein, 10 aa, artificial sequence
  HIV-1 tat protien basic region (Protein Transduction Domain)
GRKKRRQRRR

SEQ ID NO: 9 - protein, 9 aa, artificial sequence
  Polyarginine Protein Transduction Domain
RRRRRRRRR

SEQ ID NO: 10 - DNA, 30 bp, artificial sequence
  Primer designated 1F for amplifying BAC nucleic acid
atcccttagttgaacacaaaaagcacaagc

SEQ ID NO: 11 - DNA, 30 bp, artificial sequence
  Primer designated 2F for amplifying BAC nucleic acid
ttctataaagatcatccatgctaaacactg

SEQ ID NO: 12 - DNA, 28 bp, artificial sequence
  Primer designated 1R for amplifying BAC nucleic acid
tgtatgagagttttggtggttccacatc

SEQ ID NO: 13 - DNA, 25 bp, artificial sequence
  Primer designated 2R for amplifying BAC nucleic acid
gataaggactgagatatgccctggt

SEQ ID NO: 14 - DNA, 20 bp, artificial
  Forward primer for amplifying Collagen Type-1
agacatcccaccaatcacct

SEQ ID NO: 15 - DNA, 20 bp, artificial
  Reverse primer for amplifying Collagen Type-1
agatcacgtcatcgcacaac

SEQ ID NO: 16 - DNA, 21 bp, artificial
  Forward primer for amplifying Collagen Type-2
aacactgccaacgtccagatg

SEQ ID NO: 17 - DNA, 21 bp, artificial
  Reverse primer for amplifying Collagen Type-2
tcgtccagataggcaatgctg

SEQ ID NO: 18 - DNA, 19 bp, artificial
  Forward primer for amplifying Aggrecan
acgtgatcctcacggcaaa

SEQ ID NO: 19 - DNA, 22 bp, artificial
  Reverse primer for amplifying Aggrecan
gtgaaaggctcctcaggttctg

SEQ ID NO: 20 - DNA, 20 bp, artificial
  Forward primer for amplifying GAPDH
acccagaagactgtggatgg

SEQ ID NO: 21 - DNA, 20 bp, artificial
  Reverse primer for amplifying GAPDH
agaggcagggatgatgttct

SEQ ID NO: 22 - protein, 120 aa, Artificial
  Active domain of Homo sapiens GDF-6
TAFASRHGKRHGKKSRLRCSKKPLHVNFKELGWDDWIIAPLEYEAYHCEGVCDFPLRSHLEPTNHAIIQTLMNSMDPGSTPPSCCVPTKLTPISILYIDAGNNVVYKQYEDMVVESCGCR

SEQ ID NO: 23 - DNA, 363 bp, Artificial
  Nucleotide sequence encoding active domain of human GDF-6
acggccttcgccagtcgccatggcaagcggcacggcaagaagtccaggctacgctgcagcaagaagcccctgcacgtgaacttcaaggagctgggctgggacgactggattatcgcgcccctggagtacgaggcctatcactgcgagggtgtatgcgacttcccgctgcgctcgcacctggagcccaccaaccacgccatcatccagacgctgatgaactccatggaccccggctccaccccgcccagctgctgcgtgcccaccaaattgactcccatcagcattctatacatcgacgcgggcaataatgtggtctacaagcagtacgaggacatggtggtggagtcgtgcggctgcaggtag

SEQ ID NO: 24 - protein, 120 aa, Artificial; identical to SEQ ID NO: 22
  Synthetic Construct

SEQ ID NO: 25 - protein, 136 aa, Artificial
  Active domain of Homo sapiens GDF-6 fused to N-terminal FLAG tag and TEV cleavage sequence
MDYKDDDDKENLYFQGTAFASRHGKRHGKKSRLRCSKKPLHVNFKELGWDDWIIAPLEYEAYHCEGVCDFPLRSHLEPTNHAIIQTLMNSMDPGSTPPSCCVPTKLTPISILYIDAGNNVVYKQYEDMVVESCGCR

SEQ ID NO: 26 - DNA, 20 bp, artificial
  Sequence of oligonucleotide for detecting collagen-type 1
agacatcccaccaatcacct

SEQ ID NO: 27 - DNA, 20 bp, artificial
  Sequence of oligonucleotide for detecting collagen-type 1
agatcacgtcatcgcacaac

SEQ ID NO: 28 - DNA, 20 bp, artificial
  Sequence of oligonucleotide for detecting collagen-type 2
gtgacaaaggagaggctgga

SEQ ID NO: 29 - DNA, 20 bp, Artificial
  Sequence of oligonucleotide for detecting collagen-type 2
acctctagggccagaaggac

SEQ ID NO: 30 - DNA, 20 bp, artificial
  Sequence of oligonucleotide for detecting aggrecan
tcaacaacaatgcccaagac

SEQ ID NO: 31 - DNA, 20 bp, artificial
  Sequence of oligonucleotide for detecting aggrecan
aaagttgtcaggctggttgg

SEQ ID NO: 32 - DNA, 20 bp, artificial
  Sequence of oligonucleotide for detecting GAPDH
aatcccatcaccatcttcca

SEQ ID NO: 33 - DNA, 20 bp, artificial
  Sequence of oligonucleotide for detecting GAPDH
tggactccacgacgtactca

SEQ ID NO: 34 - protein, 120 aa, artificial
  retro-peptide analog of active domain of GDF-6
RCGCSEVVMDEYQKYVVNNGADIYLISIPTLKTPVCCSPPTSGPDMSNMLTQIIAHNTPELHSRLPFDCVGECHYAEYELPAIIWDDWGLEKFNVHLPKKSCRLRSKKGHRKGHRSAFAT

SEQ ID NO: 35 - protein, 136 aa, artificial
  retro-peptide analog of an active domain of GDF-6 comprising a N-terminal retro FLAG tag
RCGCSEVVMDEYQKYVVNNGADIYLISIPTLKTPVCCSPPTSGPDMSNMLTQIIAHNTPELHSRLPFDCVGECHYAEYELPAIIWDDWGLEKFNVHLPKKSCRLRSKKGHRKGHRSAFATGQFYLNEKDDDDKYDM

SEQ ID NO: 36 - protein, 120 aa, artificial; identical to SEQ ID NO: 34
  retro-inverted peptide analog of active domain of GDF-6 wherein each amino acid other than glycine is a D-amino acid

SEQ ID NO: 37 - protein, 136 aa, artificial; identical to SEQ ID NO: 35
  retroinverted peptide analog of an active domain of GDF-6 comprising a N-terminal retro FLAG tag wherein each amino acid other than glycine is a D-amino acid

(19) United States
(12) Patent Application Publication
(10) Pub. No.: US 2012/0208753 A1
(43) Pub. Date: Aug. 16, 2012

(54) Composition and Method for the Treatment or Prevention of Spinal Disorders III

(76) Inventors: Ashish Diwan, Sydney (AU); Divya Diwan, Sydney (AU)

(21) Appl. No.: 13/389,589

(22) PCT Filed: May 24, 2010
(86) PCT No.: PCT/AU10/00627

§ 371 Date: Apr. 23, 2012

(30) Foreign Application Priority Data
Aug. 25, 2009 (AU) 2009904062

(51) Int. Cl. A61K 38/18; A61M 5/00; A61M 37/00; A61P 19/00

(52) U.S. Cl. 514/8.8; 604/506; 604/500; 604/22

(57) ABSTRACT

The present invention relates to compositions comprising a modulator of GDF-6 signaling for the prevention of and/or treatment of a spinal disorder and/or spinal pain, eg., caused by and/or associated with intervertebral disc degeneration and methods of treatment of a spinal disorder and/or spinal pain comprising administering a modulator of GDF-6 signaling, or a composition comprising thereof.

### CROSS-REFERENCE TO RELATED APPLICATIONS

The application claims the benefit of priority from Australian Patent Application No. 2009904062 filed on Aug. 25, 2009, the content of which is incorporated herein in its entirety.

### FIELD OF THE INVENTION

The present invention relates to compositions of matter for the prevention of and/or treatment of a spinal disorder and/or spinal pain, e.g., caused by and/or associated with intervertebral disc degeneration and methods of treatment of a spinal disorder and/or spinal pain.

### BACKGROUND OF THE INVENTION

General

The following publications provide conventional techniques of molecular biology. Such procedures are described, for example, in the following texts that are incorporated by reference:
1) Sambrook, Fritsch & Maniatis, Molecular Cloning: A Laboratory Manual, Cold Spring Harbor Laboratories, New York, Second Edition (1989), whole of Vols I, II, and III;
2) DNA Cloning: A Practical Approach, Vols. I and II (D. N. Glover, ed., 1985), IRL Press, Oxford, whole of text;
3) Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4) Animal Cell Culture: Practical Approach, Third Edition (John R. W. Masters, ed., 2000), ISBN 0199637970, whole of text;
5) J. F. Ramalho Ortigão, “The Chemistry of Peptide Synthesis” In: Knowledge database of Access to Virtual Laboratory website (Interactiva, Germany);
6) Sakakibara, D., Teichman, J., Lien, E. Land Fenichel, R. L. (1976). Biochem. Biophys. Res. Commun. 73 336-342
7) Merrifield, R. B. (1963). J. Am. Chem. Soc. 85, 2149-2154.
8) Barany, G. and Merrifield, R. B. (1979) in The Peptides (Gross, E. and Meienhofer, J. eds.), vol. 2, pp. 1-284, Academic Press, New York.
9) Bodanszky, M. (1984) Principles of Peptide Synthesis, Springer-Verlag, Heidelberg.
10) Bodanszky, M. & Bodanszky, A. (1984) The Practice of Peptide Synthesis, Springer-Verlag, Heidelberg.

Description of Related Art

Persistent back pain poses a significant economic burden to society, mainly in terms of the large number of work days lost by patients who develop chronic back pain. The major cause of persistent back pain is intervertebral disc (IVD) degeneration. In this respect, in USA alone approximately 5.7 million people are diagnosed with IVD degeneration each year.

Intervertebral Discs (IVDs)

An IVD is a specialized connective tissue composed of a pad of fibrocartilage found between the bony vertebrae of the spine. IVDs act as a shock absorber to cushion the compressive, rotational and tensile forces applied to the vertebral column. An IVD comprises at least three elements: a tough outer tissue called the annulus fibrosus (AF) comprising concentric layers of intertwined annular bands comprising primarily collagen type I fibers; a nucleus pulposus (NP) within the AF, comprising a viscous gel containing proteoglycan and water held loosely together by an irregular network of collagen type II and elastin fibers ; and the flat, circular vertebral endplates comprising cartilage that contacts the vertebrae above and below the disc and connects to the AF. The major proteoglycan found in the NP is the glucosaminoglycan aggrecan which is high in chondroitin sulfate and keratin sulfate. This proteoglycan provides osmotic properties needed to resist compression in the disc (Adams and Roughley, Spine 31: 2151-2161, 2006). Cells of the NP are initially notochord cells that are gradually replaced during childhood by rounded cells resembling the chondrocytes of articular cartilage. Cells of the AF are fibroblast-like, elongated parallel to the collagen fibers in the AF. Cell density declines with age and is extremely low in adults, especially in the NP.

Fibrocartilage found in an IVD differs to other forms of cartilage, e.g., hyaline cartilage or elastic cartilage. For example, the fibrocartilage found in IVDs contain cartilage-1 or type-1 cartilage, whereas this form of cartilage does not occur in hyaline cartilage or elastic cartilage. Moreover, the extracellular matrix within an IVD differs from that found in other cartilage, e.g. hyaline cartilage, in so far as it contains a high proteoglycan to collagen ratio, e.g., extracellular matrix of IVD has a ratio of proteoglycan to collagen of about 27:1, whereas hyaline cartilage has a ratio of about 2:1 (Mwale et al., European Cells and Materials, 8: 58-64, 2004). The increased level of proteoglycan relative to collagen in an IVD explains to some degree the gelatinous nature of an IVD, which is required for transmitting load applied to the IVD and providing the shock absorbing nature of these organs. In contrast to IVD, other forms of cartilage, e.g., hyaline cartilage or articular cartilage operate in isolation and must retain their own shape and, as a consequence, a higher concentration of collagen to proteoglycan is desired to provide such a firm and resilient nature (Mwale et al., supra).

At the microscopic level proteoglycans of IVD extracellular matrix also differ from those of other forms of cartilage, including articular cartilage, nasal cartilage, growth plate cartilage and menesci. For example, articular cartilage nasal cartilage, growth plate cartilage and menesci contain large aggregates of proteoglycan formed from hyaluronic acid central filaments in addition to large nonaggregated monomers. In contrast to these cartilages, IVDs contain short non-aggregated proteoglycan monomers and clusters of monomers without central filaments (Buckwalter et al., J. Orthop. Res., 7: 146-151, 1989). These differences in composition of IVDs and other forms of cartilage are indicative of significant differences in collagen and/or proteoglycan metabolism between these tissues.

IVD degeneration is associated with a series of biochemical and morphologic changes that combine to alter the biomechanical properties of the disc. During IVD degeneration, the concentration of proteoglycans in the NP and the water retaining potential of the disc decrease dramatically. There are also changes in the collagen content of the NP as the synthesis of type II collagen declines and the synthesis of less tensile type I collagen increases. Another change is a shift in phenotype of the differentiated chondrocyte of the NP into a more fibrotic type.

Vertebral endplates exist at the cranial and caudal ends of each IVD separating the vertebral bone from the disc and preventing the NP from bulging into the adjacent vertebrae. Another function of the of the endplates is to absorb hydrostatic pressures resulting from the mechanical loading of the spine. Vertebral endplates are generally less than 1 mm thick and are typically thinnest in the central region adjacent to the NP. The thin central region of the endplate is also more permeable due to the presence of microscopic blood vessels, which are less common in the outer margins of the endplate. The microscopic blood vessels within the endplate provide the main source of nutrition for the inner disc.

In contrast to articular cartilage of synovial joints, the endplates are not connected directly to vertebral bone, instead being interwoven into the AF. The outer fibres of the AF (i.e. Sharpey's Fibres) are connected to the Ring Apophysis, the exposed outer periphery of the vertebral bone.

Vertebral endplates comprise osseous as well as hyaline cartilaginous components. The cartilaginous component remains throughout maturation and does not undergo ossification unlike the adjacent vertebrae. The cartilaginous component consists of a gel of hydrated proteoglycan molecules reinforced by collagen fibrils which attach to the AF.

IVD Development

In adult life, events unfolding as a consequence of injury to the disc may mimic some of the molecular events that control the development of the disc. Development of the disc is under tight molecular control both temporally and spatially. Notochordal cells are involved in the development of the spinal cord and vertebra and they also contribute towards the patterning and differentiation of the IVDs. During gastrulation, the axial mesoderm gives rise to the notochord and somites develop into two parts: a schlerotome and a dermomyotome. The cells of the schlerotome are responsible for the formation of the spine and the IVD as the schlerotomes migrate toward and around the notochord and neural tube, and later separate into areas of loosely packed cells which go on to form the NP and a densely packed cells which form the AF.

IVD Related Disorders

Kippel Feil Syndrome (KFS) is a congenital condition characterized by the fusion of two or more cervical vertebrae (Type I-III; Kaplan et al., The Spine Journal 2005 5:564-576). This abnormality is the result of a failure of proper segmentation of vertebrae in the cervical region during embryonic development (Clark et al., 1998, Pediatr Radiol 28:967-974). In KFS the IVD(s) are not developed (hypo/oligogenesis) or there is an agenesis of the disc(s). Notwithstanding that a number of de novo PAX1 missense mutations, as well as PAX1 haploinsufficiency, i.e., reduced expression of PAX1, have been associated with KFS, no definitive genetic basis for KFS has yet been identified.

Fibrodysplasia ossificans progressive (FOP) is a rare autosomal dominant disorder of connective tissue whereby patients also present with cervical spine abnormalities. FOP, a condition where there is excessive bone formation is often misdiagnosed for KFS, which has been identified by the present inventor as being an hypo/oligogenesis of the disc. Knockout mice which do not express the bone morphogenetic protein (BMP) antagonist noggin, exhibit a phenotype almost identical to FOP patients. Whilst the noggin gene (NOG) is not mutated in FOP, overactivity of the BMP pathway (i.e., enhanced BMP signaling) has been suggested as the molecular pathogenesis of FOP (e.g., in incorrect development of IVDs) (Schaffer et al., Spine 2005 30 (12): 1379-1385).

Bone Morphogenetic Proteins

BMPs are low-molecular weight glycoproteins that control many developmental processes. BMPs are multi-functional growth factors that belong to a larger family of related secreted factors, the transforming growth factor (TGF)-β superfamily. To date, around 20 BMP family members have been identified and characterized. Members of the BMP family include, for example, BMP-2, BMP-4, BMP-5, BMP-6, the osteogenic proteins OP-1 (BMP-7) and OP-2 (BMP-8), osteogenin (BMP-3), and BMP-9 to BMP-12. Other names for BMPs include growth and differentiation factors (GDF) and cartilage-derived morphogenetic proteins, e.g., CDMP-1 and -2, also known as GDF-5 and GDF-6/GDF-6, respectively. Notwithstanding that BMPs were first identified by virtue of their ability to promote ectopic cartilage and bone formation, BMP signaling plays a critical role in heart, limb, kidney, and skeletal development, and control many key steps in the formation and differentiation of the vertebrate nervous system.

BMPs signal through a molecular pathway, which is initiated by contact of extracellular BMPs with a high-affinity complex of heteromeric type II and type I serine/threonine kinase receptors. The receptor complexes in turn phosphorylate receptor regulated R-Smads 1,5 and 8 which induces them to bind Smad4 (Co-Smad) and accumulate in the nucleus where they regulate transcription. The heteromeric BMP-regulated Smad complex can bind directly, or through other transcriptional partners to BMP response elements of gene promoters of xVent2, xVent2B, Msx1, Msx2, Hex, Smad7, and Id1. The pathway is further controlled by the action of inhibitory Smads 6 and 7 and by soluble antagonists that bind extracellular BMPs inhibiting binding to heteromeric complexes such as, Noggin, Chordin and Dan.

The manner in which BMPs regulate such diverse processes is largely determined by the cellular and tissue context in which the BMP signals are received. For example, although the molecular components of BMP signaling may be highly conserved, tissue and cell-type specificity ultimately determine which BMP and combinations of receptors, intracellular mediators, and extracellular antagonists control a particular process. BMP-regulated gene expression is further controlled by interaction of Smads with tissue-specific transcription factors and cross-talk with other signalling pathways to mediate the diverse transcriptional programs associated with BMP regulated processes.

Notwithstanding our increased understanding of the molecular events involved in development of an IVD, this understanding has yet to lead to the development of an effective treatment for a spinal disorder and/or spinal pain. Rather current treatment options for a spinal disorder and/or spinal pain require surgical intervention to replace a degenerated IVD and/or remove the IVD and fuse vertebrae. In this respect, spinal fusion is expensive because it requires prolonged hospitalisation and specialist surgical expertise. Furthermore, studies suggest that in the long-term, spinal fusion actually promotes degeneration at sites adjacent to the lumbar fusion. Furthermore, replacement of the disc is a major operation and despite potential benefits, many sufferers of repeated chronic neck pain and/or back pain avoid major spinal reconstruction. It is clear from the foregoing that there remains a need for compositions and methods for the treatment of spinal disorders and/or spinal pain, e.g. a spinal disorder associated with IVD degeneration, that does not require a prolonged period of hospitalization and/or that does not aggravate the spinal disorder and/or spinal pain. Ideally, this treatment should have the potential of regenerating disc tissue and/or preventing or slowing spinal degeneration.

### SUMMARY OF INVENTION

In work leading up to the present invention, the inventors sought to identify biochemical pathway(s) that is(are) involved in the development of and/or causative of Klippel Feil Syndrome (KFS). The inventors reasoned that, because subjects suffering from KFS do not form one or more IVD(s), modulation of biochemical pathway(s) involved in pathogenesis of this disease is(are) likely to be useful for the treatment of disorders associated with IVD degeneration. By mutational analysis of a panel of subjects suffering from KFS a number of alleles of the gene encoding growth differentiation factor 6 (GDF-6) (also known as bone morphogenetic protein-13 (BMP-13) or cartilage-derived morphogenic protein-2 (CDMP-2)) associated with development of KFS were identified. Using KFS as a model of abnormal IVD development and/or maintenance, in vivo evidence that GDF-6 signaling is involved in IVD development and/or maintenance has been provided (Tassabeji et. al., Human Mutation, Accepted 6 Feb. 2008).

The inventors also reason that GDF-6 stimulates, initiates or promotes chrondrogenesis at the cost of osteogenesis during spinal development, and more particularly, that GDF-6 initiates, promotes or activates mobilization and activation of proliferative chondrocytes e.g., that remain in or have an extended proliferative phase rather than becoming hypertrophic or ossified. This implies that GDF-6 is capable of maintaining IVDs in a cartilaginous state, and/or prevents or reduces ossification from the adjacent developing vertebral bodies. In the KFS model, mutations reducing activity and/or expression of GDF-6 permit ossification from the vertebral bodies to be extended into the discal region, thereby resulting in fusion of the vertebra. This suggested to the inventors that GDF-6 expression delineates the region of the IVD th a developing fetus, wherein external tissues undergo ossification. When GDF-6 is absent or reduced, there is progressive ossification of cartilage.

The inventors have also demonstrated that recombinant GDF-6 reduced, delayed or prevented IVD degeneration and/or enhanced IVD regeneration in an accepted animal model of IVD degeneration, i.e., a sheep annular tear model of IVD degeneration.

The inventors have also demonstrated that recombinant GDF-6 induces production of extracellular matrix proteins when administered to cells that are or become incorporated into an IVD, e.g., collagen type-1 and collagen type-2 and of a transcription factor involved in extracellular matrix synthesis, i.e., SOX-9. These results indicate that GDF-6 induces biological changes within IVD cells associated with IVD regeneration.

Moreover, the inventors have shown that, against conventional wisdom, the end-plate of an IVD provides a source of cells that may replenish avascular discal cells. Previously, the end-plate was considered to be similar to cartilage at the end of long bones e.g., a femur, comprising cartilage-like cells within a hard cartilaginous matrix that acts as a diffusion barrier. The inventors have found that exogenous administration of GDF-6 within nucleus pulposus, or alternatively, administration of GDF-6 in and/or adjacent an end-plate such as in a region of ring apophysis and/or in a region of sub-chondral bone, can lead to mobilization, activation or proliferation of the end-plate cells to replenish avascular discal cells. For example, mature cells can be “discharged” from the end-plate region into an IVD. The annulus-bone junction and apophyseal ring in human adults also contributes to this cellular renewal process. Thus, one or more morphogens such as GDF-6 or a modulatory compound thereof or a downstream effector of GDF-6 contributes to the maintenance and regeneration of an IVD, or prevention of IVD degeneration by reducing, delaying or preventing ossification in the IVD and/or promoting cellular renewal and/or chondrogenesis. This provides the means for activating cells such as stem cells that give rise to cells participating in IVD repair processes, and/or preventing ossification of IVDs, via injection of GDF-6 or a modulatory compound thereof or a downstream effector of GDF-6. Accordingly, the invention encompasses a method of stimulating cells in or adjacent the end-plate, including resting/precursor/stem cells, and/or mobilizing cells from structures adjacent to the IVD e.g., in subchondral bone and/or apophyeal ring, wherein stimulation or mobilization of the cells increases cellularity and/or functionality of the IVD.

The examples provided herein demonstrate that administration of GDF-6 into the IVD leads to one or more of: (i) an increase in proliferation of resting or stem cells in the region of the end-plate in vivo; (ii) active mobilization of multiple layers of cells on end-plates into disc tissue in vivo; (iii) suppressed or absent or undetectable neovascularization of the end-plate in vivo; (iv) restoration of the viscoelastic properties of an IVD in vivo e.g., as determined by MRI; (v) differentiation of BMSC into nucleus pulposus (NP) cells or NP-like cells in vivo as determined by expression of one or more markers of NP cells; and (vi) promotion of chondrogenesis in vivo as determined by expression of one or more markers associated with chondrogenesis. Moreover, the examples provided herein demonstrate dose-responsive enhancement in expression of markers associated with chondrogenesis, especially Sox9, and dose-responsive enhancement in expression of collagen I and collagen II.

The inventors extended these studies by modulating the level of proteins involved in GDF-6 signaling, e.g., a transcription factor, such as MSX-1 and/or MSX-2, in primary cells isolated from an IVD. As exemplified herein, over-expression of MSX-1 and/or MSX-2 in a cell isolated from an IVD, e.g., an annulus fibrosus cell or a nucleus pulposus cell, results in increased collagen production and increased extra-cellular matrix production by the cell. Both increased collagen production and increased extra-cellular matrix production by cells of the IVD are associated with IVD regeneration. Accordingly, a composition that modulates GDF-6 signaling is an attractive therapeutic for treating a spinal disorder and/or spinal pain, e.g., a spinal disorder and/or spinal pain associated with IVD degeneration.

In this respect, as discussed herein, the present inventors have demonstrated that modulation of various components of a GDF-6 signaling pathway in an IVD or a cell or tissue thereof is useful for treating a spinal disorder and/or spinal pain. Without limiting the invention in any manner, such modulation may comprise increasing the level and/or activity of GDF-6 in an IVD or a cell or tissue thereof and/or increasing the level and/or activity of MSX-1 in an IVD or a cell or tissue thereof and/or increasing the level and/or activity of MSX-2 in an IVD or a cell or tissue thereof. However, the present invention also encompasses the modulation of any component of GDF-6 signaling in an IVD or a cell or tissue thereof. In this respect, GDF-6 binds to a dimeric receptor comprising BMP receptor (BMPR)-1A (also known as ALK3) and/or a BMPR-1B (also known as ALK-6) and/or a BMPR-II. Following binding of GDF-6 to the receptor, the activated receptor phosphorylates a receptor-mediated Smad, e.g., Smad-1 and/or Smad-5 and/or Smad-8, which then forms a complex with Smad-4. The complex comprising a receptor-mediated Smad and Smad-4 then translocates to the nucleus and activates expression of a GDF-6-regulated gene, such as, for example, MSX-1 and/or MSX-2.

The term “modulator of GDF-6 signaling” is to be understood to mean a compound that modulates any component of a GDF-6 signal transduction pathway in an IVD or a cell or tissue thereof, e.g., as described in the previous paragraph, including GDF-6 itself and/or a GDF-6 regulated gene, e.g., MSX-1 or MSX-1. This term also encompasses a compound that modulates, for example, activation of BMPR-1A and/or BMPR-IB and/or BMPR-II and/or Smad-1 and/or Smad-5 and/or Smad-8 and/or Smad-4. Similarly, the term “modulating GDF-6 signaling” shall be taken to mean modulating any component of a GDF-6 signal transduction pathway in an IVD or a cell or tissue thereof, e.g., GDF-6 and/or MSX-1 and/or MSX-2 and/or BMPR-1A and/or BMPR-IB and/or BMPR-II and/or Smad-1 and/or Smad-5 and/or Smad-8 and/or Smad-4.

As used herein, the term “modulator” shall be taken to mean a compound that enhances or reduces the activity or amount of GDF-6 signaling in an IVD or a cell or tissue thereof. In one example, the modulator enhances GDF-6 signaling in an IVD or a cell or tissue thereof.

The inventors have also produced methods and devices for administering a modulator of GDF-6 signaling to an IVD in such a manner that it is applied to a plurality of sites within the IVD and/or within a nucleus pulposus and/or within a region of the IVD defined by an annulus fibrosus and/or adjacent to at least a portion of a nucleus pulposus. In this respect, the viscous nature of the IVD, e.g., the nucleus pulposus means that a composition of matter administered by a single bolus injection may not disperse or may not be distributed within the IVD or nucleus pulposus and, as a consequence, may not exert sufficient biological effect to provide a therapeutic benefit. By administering a modulator of GDF-6 signaling, the inventors facilitate dispersion or distribution of the modulator within the IVD, preferably within the nucleus pulposus thereby enhancing the therapeutic benefit provided by the modulator.

Methods and devices of the invention also provide for application of the modulator or composition more generally to a region of an IVD that permits the modulator or composition to mobilize, activate or proliferate cells in and/or adjacent an end-plate or enhance mobilization, activation or proliferation of said cells. This means that direct administration to the nucleus pulposus is not an absolute requirement. For example, the modulator or composition may be administered by intradiscal injection wherein the IVD is accessed and the modulator or composition is injected percutaneously, e.g., under fluoroscopic or sterotactic image guidance, and diffuses to the end-plates to exert an effect. Alternatively, the modulator or composition may be administered by osseous vascular injection/implantation, wherein access to an end-plate is via a bony opposing face thereof and the modulator or composition is injected directly into that bony element or a venous sinusoid of the vertebral body e.g., by virtue of a trocar of sufficient strength, stiffness and sharpness to make a hole in the bone and/or by virtue of a cannula targeted very close to the bony side of the end plate which dispenses the modulator or composition to a plurality of sites via multiple exit ports or openings. Alternatively, the modulator or composition may be administered by boring through vertebral bodies and one or more IVDs such as by a trans-sacral route. Alternatively, the modulator or composition may be administered by continuous discharge e.g., by means of a pump or a long-acting depot injected into the IVD or adjacent bone by a cannula. Alternatively, the modulator or composition may be administered by peri-annular infiltration or by means of a catheter that is negotiated into the epidural space from one side to contralateral side within the canal close to the annulus whilst still in the epidural space. Alternatively, the modulator or composition may be administered parenterally, with or without an attractant injected intradiscally. Alternatively, the modulator or composition may be administered by cutaneous application such as by transdermal patch. The use of oral formulations is also contemplated by the present invention.

### Specific Embodiments

The scope of the invention will be apparent from the claims as filed with the application that follow the examples. The claims as filed with the application are hereby incorporated into the description. The scope of the invention will also be apparent from the following description of specific embodiments and/or detailed description of preferred embodiments.

In one example, the present invention provides a method for preventing or delaying or treating a spinal disorder and/or spinal pain in a subject, said method comprising administering a modulator of GDF-6 signaling or composition comprising a modulator of GDF-6 signaling to a subject suffering from a spinal disorder and/or spinal pain for a time and under conditions sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject.

As used herein, the term “adjacent” means in contact with or sufficiently close to a stated integer so as to exert a desired and stated effect on the integer. Accordingly, cells adjacent an end-plate may be in a region of ring apophysis and/or in a region of sub-chondral bone or other bone-comprising surface adjacent an end-plate.

Cells in and/or adjacent an end-plate may be resting or quiescent in the absence of the administered GDF-6. Alternatively, or in addition, the cells in and/or adjacent an end-plate are self-renewing in the absence of the administered GDF-6. Alternatively, or in addition, the cells in and/or adjacent an end-plate are uncommitted in the absence of the administered GDF-6. Alternatively, or in addition, the cells in and/or adjacent an end-plate are stem cells, such as resting stem cells or quiescent stem cells.

The mobilized, activated, or proliferating cells are incorporated into IVD before, concomitant with, or following their mobilization and/or activation and/or proliferation. Preferably, the cells are incorporated into damaged IVD following their mobilization and/or activation and/or proliferation. Alternatively, or in addition, mobilization, activation, proliferation, enhanced mobilization, enhanced activation or enhanced proliferation of the cells in and/or adjacent an end-plate stimulates or enhances chondrogenesis of the cells and/or and/or commitment of the cells to chondrogenesis. Chondrogenesis or commitment to chondrogenesis may be before, concomitant with, or following mobilization and/or activation and/or proliferation of the cells. Alternatively, or in addition, mobilization, activation, proliferation, enhanced mobilization, enhanced activation or enhanced proliferation of cells in and/or adjacent an end-plate stimulates or enhances proteoglycan production by the cells. Alternatively, or in addition, mobilization, activation, proliferation, enhanced mobilization, enhanced activation or enhanced proliferation of cells in and/or adjacent an end-plate stimulates or enhances collagen production by the cells, e.g., cells in the region of an end-plate.

In another example, the method as described according to any example hereof further comprises monitoring efficacy of therapy e.g., to determine effective therapy. For example, therapy is monitored by determining one or more markers associated with chondrogenesis, wherein an increased level of said one or more markers in an IVD is indicative of effective therapy. Markers associated with chondrogenesis include, individually or collectively, one or more proteins of the chondrogenic pathway e.g., protein(s) selected from the group consisting of Runx2, Sox9, Noggin, chordin, Msx-1, Msx-2, BMP-2 and BMP-4 and combinations thereof. Alternatively, or in addition, markers associated with chondrogenesis include, individually or collectively, one or more nucleic acids encoding protein(s) of the chondrogenic pathway such as those infra. Alternatively, or in addition, efficacy of therapy is monitored by determining neovascularization in and/or adjacent the end-plate, wherein absence of neovascularization or absence of enhanced neovascularization an and/or adjacent the end-plate is indicative of effective therapy. Alternatively, or in addition, efficacy of therapy is monitored by determining mobilization, activation or proliferation of cells in and/or adjacent the end-plate, wherein mobilization, activation, proliferation, enhanced mobilization, enhanced activation or enhanced proliferation of cells in and/or adjacent the end-plate, is indicative of effective therapy. Alternatively, or in addition, efficacy of therapy is monitored by determining expression and/or increased expression of one or more proteins regulated by GDF-6 in an IVD, wherein an increased level of said expression is indicative of effective therapy. Exemplary proteins regulated by GDF-6 that are useful for such monitoring are selected e.g., from the group consisting of: Runx2, Sox9, Noggin, chordin, Msx-1, Msx-2, BMP-2 and BMP-4 and combinations thereof. Alternatively, or in addition, efficacy of therapy is monitored by determining increased expression of one or more genes regulated by GDF-6 in an IVD, wherein an increased level of said expression is indicative of effective therapy. Exemplary genes regulated by GDF-6 that are useful in this context include genes that encode proteins regulated by GDF-6 such as those described infra. Alternatively, or in addition, efficacy of therapy is monitored by determining increased expression of Sox9 in annulus fibrosis cells, wherein increased expression is indicative of effective therapy. Alternatively, or in addition, efficacy of therapy is monitored by determining proteoglycan in an IVD, wherein an increased level of proteoglycan in an IVD is indicative of effective therapy. Alternatively, or in addition, efficacy of therapy is monitored by determining collagen I and/or collagen II in an IVD, wherein an increased level of collagen I and/or collagen II in an IVD is indicative of effective therapy. For example, increased collagen I and/or collagen II may be in end-plate cells. Alternatively, or in addition, efficacy of therapy is monitored by assessing one or more physical properties of the IVD e.g., restoration of viscoelastic properties of the IVD and/or IVD compression and/or IVD height such as by MRI or X-ray, wherein increased viscoelasticity or IVD height or reduced compression is indicative of effective therapy. Alternatively, or in addition, efficacy of therapy is monitored by assessing the degree of pain experienced by the subject over time, wherein reduced pain is indicative of effective therapy. Alternatively, or in addition, efficacy of therapy is monitored by assessing the patient's spine mobility over time, wherein increased range of motion is indicative of effective therapy.

In another example, the method of the present invention as described according to any example hereof may comprise administering the modulator or composition to a plurality of sites within an IVD and/or a plurality of sites within a nucleus pulposus and/or a plurality of sites adjacent to at least a portion of a nucleus pulposus and/or a plurality of sites within a region of an IVD defined by the internal wall of an annulus fibrosus. This encompasses administration of the composition or modulator to a plurality of sites in a single administration. Alternatively, or in addition, the modulator or composition is administered in a patterned manner. For example, the modulator or composition is administered to a plurality of sites or in a patterned manner so as to permit said modulator or composition to disperse or distribute evenly throughout the nucleus pulposus.

In another example, the modulator or composition is administered via a medical device comprising a delivery conduit having a proximal end attachable to a source of the modulator of GDF-6 signaling or the composition and an emitter structure at a distal end of the delivery conduit, wherein the emitter structure defines a plurality of spaced discharge apertures through which the modulator or composition is delivered.

In another example, the modulator or composition is administered by injection through one or more sites in bone and in sufficient proximity to an end-plate in or adjacent an IVD in need of treatment such that the modulator or composition is capable of mobilizing, activating, proliferating, enhancing mobilization, enhancing activation or enhancing proliferation of cells in and/or adjacent the end-plate of the IVD in need of treatment. For example, the modulator or composition can be administered by injection to a single site below the end-plate in or adjacent an IVD in need of treatment.

Alternatively, or in addition, the modulator or composition is administered to a plurality of sites. This encompasses administering the modulator or composition by injection through a plurality of sites in bone wherein each of said sites is in sufficient proximity to an end-plate in or adjacent an IVD in need of treatment such that the modulator or composition is capable of mobilizing, activating, proliferating, enhancing mobilization, enhancing activation or enhancing proliferation of cells in and/or adjacent an end-plate of each IVD in need of treatment. Alternatively, this encompasses administering the modulator or composition by injection through one or a plurality of sites in bone using a medical device comprising a delivery conduit having a proximal end attachable to a source of the modulator or composition and an emitter structure at a distal end of the delivery conduit, wherein the emitter structure defines a plurality of spaced discharge apertures through which the modulator or composition is delivered, such that the number of injection sites in bone is less than the number of IVDs in need of treatment. For example, the modulator or composition is administered by injection through a single site in bone and dispering the modulator or composition to a plurality of IVDs in need of treatment.

In another example, the present invention provides a method for preventing or delaying or treating a spinal disorder and/or spinal pain in a subject, said method comprising administering a modulator of GDF-6 signaling or composition comprising a modulator of GDF-6 signaling to a subject suffering from a spinal disorder and/or spinal pain for a time and under conditions sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject, wherein said administration comprises:
(i) accessing a region of an IVD by surgical intervention or injection;
(ii) providing or obtaining a medical device comprising the modulator or composition wherein the medical device comprises a delivery conduit having a proximal end attachable to a source of the modulator of GDF-6 signaling or the composition and an emitter structure at a distal end of the delivery conduit, wherein the emitter structure defines a plurality of spaced discharge apertures through which the modulator or composition is delivered;
(iii) inserting the emitter structure of the medical device at least partially into the accessed region of the IVD;
(iv) manipulating the emitter structure so that the emitter structure is positioned within the IVD and/or at least partially surrounds or is positioned within the nucleus pulposus and/or a region of the IVD defined by an internal wall of the annulus fibrosus; and
(v) discharging the modulator or composition through the apertures of the device so as to administer said modulator or composition to a plurality of sites within the IVD in a single administration and/or at least partially surrounds or is positioned within the nucleus pulposus and/or a region of the IVD defined by an internal wall of the annulus fibrosus, thereby administering the modulator or composition to the subject.

In another example, the present invention provides a method for preventing or delaying or treating a spinal disorder and/or spinal pain in a subject, said method comprising administering a modulator of GDF-6 signaling or composition comprising a modulator of GDF-6 signaling to a subject suffering from a spinal disorder and/or spinal pain for a time and under conditions sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject, wherein said administration comprises providing or obtaining an agent delivery system that comprises:
(i) a dispenser defining a reservoir and an outlet port in communication with the reservoir;
(ii) a high density, immiscible, non-reactive, biocompatible displacement fluid comprising the modulator or composition, said fluid being contained within the reservoir; and
(iii) a displacement device arranged in the reservoir for displacing the fluid through the outlet port of the dispenser.

The agent delivery system may comprise a receptacle for the fluid, the receptacle having a mounting formation for mounting the receptacle to the dispenser so that an interior of the receptacle is in communication with the outlet port of the dispenser. The receptacle may comprise a cannula with at least one discharge opening. For example, the cannula may be elongate having a side wall defining a plurality of axially spaced discharge openings such as an arrangement wherein each discharge opening includes an occluding device for inhibiting back flow of the fluid into the interior of the cannula or an arrangement wherein each of at least some of the openings open out into a recessed region of the side wall of the cannula. Alternatively, or in addition, the invention comprises use of an agent delivery system wherein the cannula is shaped and dimensioned to access a plurality of sites simultaneously and/or wherein the cannula is flexible to be able to be directed to a desired location in a patient's body. The agent delivery system may also comprises a reaming tool for forming a passage through bone at a site in the patient's body into which the receptacle is to be inserted. For example, the reaming tool may be steerable.

In another example, the present invention provides a method for preventing or delaying or treating a spinal disorder and/or spinal pain in a subject, said method comprising administering a modulator of GDF-6 signaling or composition comprising a modulator of GDF-6 signaling to a subject suffering from a spinal disorder and/or spinal pain for a time and under conditions sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject, wherein said administration comprises providing or obtaining an agent delivery system that comprises:
(i) an elongate body defining a lumen;
(ii) at least one opening defined in the body through which the modulator or composition can be discharged; and
(iii) an occluding device contained in a receptacle in register with at least one of said openings, said occluding device being for closing off the opening(s) to thereby inhibit back flow of the modulator or composition into the lumen of the body after being discharged through the opening(s).

The body may comprise a mounting formation for mounting to a dispenser so that an interior of the body is in communication with an outlet port of the dispenser. The body may also comprise a cannula, e.g., a cannula having a side wall defining a plurality of axially spaced discharge openings. In one arrangement, a proportion of the plurality of openings open out into a recessed region of the side wall of the cannula. It is to be understood in this context that the cannula according top any arrangement described herein may be shaped and dimensioned to access a plurality of sites simultaneously and/or it may be flexible to permit it to be directed to a desired location in a patient's body.

Another example of the present invention provides a method for preventing or delaying or treating a spinal disorder and/or spinal pain in a subject, said method comprising administering a modulator of GDF-6 signaling or composition comprising a modulator of GDF-6 signaling to a subject suffering from a spinal disorder and/or spinal pain for a time and under conditions sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject, wherein said administration comprises providing or obtaining a reaming tool for forming a passage in bone in a patient's body, the reaming tool comprising:
(i) a reaming head;
(ii) a pivot to which the reaming head is pivotally mounted; and
(iii) a steering mechanism for steering the reaming head through body tissue and bone.

The reaming head may be omni-directionally pivotally mounted relative to the pivot.

Another example of the present invention provides a method for preventing or delaying or treating a spinal disorder and/or spinal pain in a subject, said method comprising administering a modulator of GDF-6 signaling or composition comprising a modulator of GDF-6 signaling to a subject suffering from a spinal disorder and/or spinal pain for a time and under conditions sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject, wherein said administration comprises:
(i) inserting a cannula comprising the modulator or composition into a site in the vertebral column of the subject, wherein the cannula is mounted on a dispensing device; and
(ii) using a high density, immiscible, non-reactive, biocompatible displacement fluid contained within a reservoir of the dispensing device to discharge the modulator or composition from the cannula.

The cannula may be inserted into the patient's body percutaneously to thereby access the site of insertion into the vertebral column of the subject. This may mean forming a passage through tissue and bone. For example, a passage may be formed through one or more vertebrae on at least one side of an IVD to be treated and delivering the modulator or composition such that it is capable of mobilizing, activating, proliferating, enhancing mobilization, enhancing activation or enhancing proliferation of cells in and/or adjacent an end-plate. The modulator or composition may also be delivered by injection through a number of vertebrae simultaneously.

Alternatively, the cannula may be inserted into the patient's body trans-sacrally.

Alternatively, the cannula may be inserted into the patient's body peri-annularly e.g., adjacent an IVD in need of treatment. Peri-annular insertion of the cannula may comprise a mode of insertion selected from the group consisting of: trans-sacral epidural insertion, transforaminal epidural insertion and interlaminar periannular insertion. Alternatively, or in addition, peri-annular insertion of the cannula may comprise negotiating the cannula through the epidural space from one side to a contralateral side within the spinal canal close to an annulus of an IVD and negotiating the cannula in extra-canal space in the periannular area.

Alternatively, the cannula may be inserted into the patient's body by a method comprising manipulating the cannula about cartilaginous tissue in the patient's body.

It is to be understood that the method of the present invention as described in any example hereof comprises use of any modulator of GDF-6 signaling, e.g., a modulator that modulates the activity and/or expression of a molecule selected from the group consisting of GDF-6, MSX-1, MSX-2, BMPR-1A, BMPR-IB, BMPR-II, Smad-1, Smad-5, Smad-8, Smad-4 and mixtures thereof. The modulator may be a peptide or polypeptide such as GDF-6 or an active fragment thereof or an analog thereof or a derivative thereof. Alternatively, the modulator may be a peptide or polypeptide such as MSX-1 or an active fragment thereof or an analog thereof or a derivative thereof. Alternatively, the modulator may be a peptide or polypeptide such as MSX-2 or an active fragment thereof or an analog thereof or a derivative thereof.

An exemplary analogue of GDF-6 comprises about 120 amino acids derived from the C-terminal portion of full-length GDF-6 polypeptide, including such fragments having approximately the same bioactivity as full-length recombinant GDF-6 e.g., as determined by ability to induce alkaline phosphatase activity in cells. Analogues of GDF-6 having the appropriate signaling activity may comprise a sequence of a fragment of full-length GDF-6 without the pro-region of the GDF-6 polypeptide. Preferred analogues will comprise an N-terminal methionine residue.

In performing the method of the present invention according to any example hereof, it is to be understood that the modulator or composition may be administered as isolated protein, a pharmaceutical formulation, or by means of a cell comprising and/or expressing the modulator of GDF-6 signaling. For example, the modulator may be administered by means of a stem cell expressing the modulator.

As used herein, the term “IVD degeneration” shall be taken to mean a process in which an amount of extracellular matrix and/or water is reduced in an IVD characterized by one or more of the following:
(i) a reduced height (i.e., the distance between the edges of the disc located between two vertebrae is reduced), e.g., relative to disc in a normal and/or healthy subject;
(ii) a reduced proteoglycan level in an IVD, e.g., relative to a proteoglycan level in an IVD in a normal and/or healthy subject;
(iii) a reduced water content, e.g., relative to a water content in an IVD in a normal and/or healthy subject;
(iv) a reduced level of Type II collagen and/or a Type IX collagen in an IVD, e.g., relative to the level of a Type II collagen and/or a Type IX collagen in an IVD normal and/or healthy individual;
(v) an enhanced level of a Type III collagen and/or a Type VI collagen in an IVD, e.g., relative to the level of a Type III collagen and/or a Type VI collagen in an IVD normal and/or healthy individual;
(vi) an increased number of apoptotic cells and/or fewer cells in an IVD, e.g., relative to the number of apoptotic cells or the number of cells in an IVD normal and/or healthy individual; and
(vii) structural failure of an IVD, such as, for example, a radial fissure, disc prolapse, end-plate damage, internal collapse of the annulus or external collapse of the annulus.

Notwithstanding that several of the characteristics discussed in the previous paragraph may be determined by comparing the level of a characteristic to the same characteristic in an IVD in a normal and/or healthy subject, such a direct comparison need not necessarily be performed. Rather, the level of the characteristic may be compared to, for example, a data set containing information pertaining to that characteristic derived from a population of normal and/or healthy individuals.

As used herein, the term “IVD regeneration” shall be taken to mean that one or more characteristics of IVD degeneration (e.g., as described supra) is partially or completely reversed. For example, following treatment with a modulator of GDF-6 signaling one or more of the characteristic described herein above is the same or similar level to that in an IVD in a normal and/or healthy individual.

The term “amount” as used herein is not be taken to mean exclusively a specific quantity, e.g., weight of a modulator and/or any specific number of cells expressing and/or comprising a modulator. Unless the context requires otherwise, the term “amount” shall be taken to mean an amount that is at least sufficient to accomplish a stated purpose e.g., sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject. Methods for determining mobilization, activation or proliferation of cells in and/or adjacent an end-plate will be apparent to the skilled artisan and/or described herein.

It will be apparent from the preceding description, that the present invention clearly extends to any use of an amount of a modulator of GDF-6 signaling in the manufacture of a medicament for the treatment of a spinal disorder and/or spinal pain and/or intervertebral disc degeneration in a subject, wherein the amount of a modulator of GDF-6 signaling is sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject. For example, the medicament may comprises a suitable carrier or excipient having a viscosity that permits the medicament to disperse or distribute evenly throughout the nucleus pulposus of a subject. In accordance with such a use, it will also be apparent that the modulator of GDF-6 signaling induces or enhances GDF-6 signaling in an intervertebral disc or a cell or tissue thereof. For example, the modulator of GDF-6 signaling may modulate the activity and/or expression of a molecule selected from the group consisting of GDF-6, MSX-1, MSX-2, BMP receptor (BMPR)-1A, BMPR-IB, BMPR-II, Smad-1, Smad-5, Smad-8, Smad-4 and mixtures thereof. It will also be apparent from the preceding description that the modulator may be a peptide or polypeptide such as, for example, a GDF-6 polypeptide or an active fragment thereof or an analog thereof or a derivative thereof or a cell expressing or comprising said GDF-6 polypeptide or active fragment thereof or analog or a derivative, or an MSX-1 polypeptide or an active fragment thereof or an analog thereof or a derivative thereof or a cell expressing or comprising said MSX-1 polypeptide or active fragment thereof or analog or a derivative, or an MSX-2 polypeptide or an active fragment thereof or an analog thereof or a derivative thereof or a cell expressing or comprising said MSX-2 polypeptide or active fragment thereof or analog or a derivative. Exemplary analogues of GTDF-6 are those described infra.

In another example, the present invention provides a composition for modulating GDF-6 signaling in an intervertebral disc or a cell or tissue thereof sufficient to reduce, delay or prevent intervertebral disc degeneration in a subject and/or to induce and/or enhance intervertebral disc regeneration in a subject, said composition comprising (i) an amount of a modulator of GDF-6 signaling sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject; (ii) a suitable carrier or excipient; and (iii) instructions for administering the composition to an intervertebral disc of a subject. The composition may comprise a pharmaceutical formulation or a stem cell comprising or expressing a modulator of GDF-6 signaling. Pharmaceutical formulations and/or stem cells will generally comprise an amount of a polypeptide modulator of GDF-6 signaling sufficient to achieve the desired physiological effect e.g., enhanced cellularity of the IVD. Pharmaceutical formulations may be slow release compositions and/or have a viscosity that permits it to disperse or distribute evenly throughout the nucleus pulposus of an IVD.

As used herein, the term “suitable carrier or excipient” shall be taken to mean a compound or mixture thereof that is suitable for administration to a subject for the treatment of a spinal disorder and/or spinal pain, albeit not necessarily limited in use to that context.

In one example, a suitable carrier or excipient is a “carrier or excipient for in situ administration”. In this respect, a “carrier or excipient for in situ administration” shall be taken to mean a compound or mixture thereof that is suitable for administration to an IVD or a region surrounding an IVD in a subject.

In another example, a suitable carrier or excipient is an intraspinal carrier or excipient. As used herein, the term “intraspinal carrier or excipient” shall be taken to mean a compound or mixture thereof that is described in the art only with reference to administration into a spine.

In a still further example, a suitable carrier or excipient is an intra-IVD carrier or excipient. The term “intra-IVD carrier or excipient” shall be taken to mean a compound or mixture thereof that is suitable for application into an IVD, and which may be suitable for use in other contexts.

Preferred carriers or excipients are suitable for administration by injection into an IVD or alternatively, by direct application to an IVD.

A carrier and excipient useful in a composition described herein according to any example will generally not inhibit to any significant degree a relevant biological activity of the active compound e.g., the carrier or excipient will not significantly inhibit the activity of the active compound with respect to modulation of GDF-6 signaling and/or IVD degeneration and/or IVD regeneration. For example, the carrier or excipient provides a buffering activity to maintain the compound at a suitable pH to thereby exert its biological activity.

In another example, a carrier or excipient in a composition comprising a GDF-6 polypeptide or active fragment or analog or derivative thereof permits the GDF-6 polypeptide, active fragment, analog or derivative to form a dimer and/or to remain in a dimeric state, i.e., the carrier or excipient is non-reducing.

Alternatively, or in addition, a suitable carrier or excipient permits a cell, e.g., a stem cell, to survive and/or grow. In one example, a suitable carrier or excipient promotes or enhances growth of a cell, e.g., a stem cell.

In one example, the composition has a viscosity that permits it to disperse or distribute evenly throughout the nucleus pulposus of a subject.

Alternatively, or in addition, the carrier or excipient comprises a compound that enhances cellular uptake of a modulator of GDF-6 signaling. For example, a carrier or excipient comprises a liposome to facilitate cellular uptake. In another example, a carrier or excipient for a nucleic acid modulator of GDF-6 signaling comprises a lipid-based delivery agent, e.g., 2, 3-dioleyloxy-N-[2(sperminecarboxyamido)ethyl]-N,N-dimethyl-1-propanaminium trifluoroacetate, which is sold commercially as Lipofectamine 2000 (Invitrogen). Other lipid-based delivery agents will be apparent to the skilled artisan and/or described herein.

Alternatively, or in addition, the carrier or excipient comprises a compound that enhances the activity of modulator of GDF-6 signaling and/or reduces inhibition of a modulator of GDF-6 signaling, e.g., a protease inhibitor and/or a DNase inhibitor and/or a RNase inhibitor to thereby enhance the stability of the modulator.

Additional suitable carriers include, for example, collagen type I or collagen type II, e.g., of cervical or lumbar origin, recombinant elastin, hyaluronic acid, a polysaccharide, a chitin derivative, polyurethane foam, poly-lactic acid (PLA) polymer), poly-glycolic acid (PGA, PLGA) amongst others.

A preferred carrier or excipient is liquid at room temperature, e.g., at about 23° C., and becomes more viscous at body temperature, e.g., at about 37° C. The liquid nature of such a carrier or excipient facilitates administration of a composition as described herein according to any example to or within an IVD and/or to or within a nucleus pulposus and/or to or within a region of an IVD defined by an annulus fibrosus. Following administration, the carrier or excipient becomes more viscous thereby retaining the modulator of GDF-6 signaling at a site within a subject for a time and under conditions sufficient for the modulator to exert a beneficial effect, e.g., to modulate GDF-6 signaling and to reduce, prevent or delay IVD degeneration and/or to enhance or induce IVD regeneration. Preferably, the carrier or excipient has a stiffness of from about 1 Mpa to about 2 Mpa at about 37° C., e.g., to provide support to an IVD.

In one preferred example, a composition as described herein according to any example comprises an amount of a modulator of GDF-6 signaling sufficient to induce or enhance collagen synthesis in an IVD cell, e.g., an annulus fibrosus cell and/or a nucleus pulposus cell and/or to enhance collagen in an IVD. Preferably, the composition comprises an amount of a modulator of GDF-6 to enhance synthesis of collagen-1 or collagen-2 in an IVD cell, e.g., an annulus fibrosus cell and/or a nucleus pulposus cell and/or to enhance collagen-1 and/or collagen-2 in an IVD.

In another example, a composition as described herein according to any example comprises an amount of a modulator of GDF-6 signaling sufficient to induce or enhance expression of SOX9 an IVD cell, e.g., an annulus fibrosus cell and/or a nucleus pulposus cell.

In one example, the composition additionally comprises a radio-opaque composition, such as, for example, 5-(acetyl-(2,3-dihydroxypropyl)amino)-N,N′-bis(2,3-dihydroxypropyl)-2,4,6-triiodo-benzene-1,3-dicarboxamide (e.g., Omnipaque®), 3,5-diacetamido-2,4,6-triiodobenzoate, BaSO₄, or a composition as described in U.S. Pat. No. 6,635,064. Such a radio-opaque composition permits detection of the composition, e.g., to determine the distribution of the composition within an IVD, e.g., within or adjacent to at least a portion of a nucleus pulposus.

In another example, the composition of the present invention comprises an additional composition of matter having synergistic activity with respect to the active modulator of GDF-6 signaling in so far as inhibiting or preventing or delaying IVD degeneration and/or enhancing or inducing IVD degeneration is concerned e.g., a stem cell.

Alternatively, or in addition a composition as described herein according to any example comprises an additional compound, such as, for example, morphogenetic protein to enhance regeneration of an IVD and/or prevent or reduce or delay IVD degeneration. Alternatively, or in addition, a composition as described herein according to any example additionally comprises a mitogen, such as, for example, insulin-like growth factor-1 (IGF-1) and/or epidermal growth factor (EGF) and/or fibroblast growth factor (FGF). Alternatively, or in addition, a composition as described herein according to any example additionally comprises an anti-catabolic compound, such as, for example, an inhibitor of a matrix-metalloproteinase, e.g., tissue inhibitor of matrix metalloproteinase-1 (TIMP-1). Suitable additional compounds will be apparent to the skilled artisan based on the description herein.

In a further example, a composition as described herein according to any example additionally comprises an analgesic and/or an anti-inflammatory composition.

The skilled artisan will be aware that a composition as described herein according to any example may be in a variety of forms, such as, for example, a liquid or a gel or a matrix or a lyophilized composition.

In another example, the present invention provides a method for producing a composition for modulating GDF-6 signaling in an intervertebral disc or a cell or tissue thereof to thereby reduce, delay or prevent intervertebral disc degeneration in a subject and/or to induce and/or enhance intervertebral disc regeneration in a subject, said method comprising mixing or otherwise combining an amount of a modulator of GDF-6 signaling sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject and a suitable carrier or excipient and optionally, providing instructions for administering the combination to an intervertebral disc of a subject. As will be apparent from the preceding description, an exemplary carrier or excipient has a viscosity that permits the composition to disperse or distribute evenly throughout the nucleus pulposus of an IVD.

The present invention also provides a medical device for forming a method of the as described according to any example hereof. The device may be a device as represented in any one of FIGS. 8 to 18.

### DEFINITIONS

This specification contains nucleotide and amino acid sequence information prepared using PatentIn Version 3.4, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (e.g. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example described herein is to be applied mutatis mutandis to each and every other example unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

### BRIEF DESCRIPTION OF THE DRAWINGS

FIG. 1, Panel a is a is a copy of a photomicrograph showing staining of a rat vertebral section from rat for GDF-6 immunoreactivity. GDF-6 is detected in nucleus pulposus cells of the rat and within the growth plate of the vertebrae.

FIG. 1, Panel b is a copy of a photomicrograph showing staining of a section from an IVD of a rat for GDF-6 immunoreactivity. GDF-6 is detected in nucleus pulposus cells.

FIG. 1, Panel c is a copy of a photomicrograph showing staining of a section from an IVD of a human for GDF-6 immunoreactivity. GDF-6 is not detected in annular fibrosus cells.

FIG. 1, Panel d is a copy of a photomicrograph showing staining of a section from an IVD of a human for GDF-6 immunoreactivity. GDF-6 is detected in nucleus pulposus cells.

FIG. 2 is a graphical representation showing the level of H-proline incorporation into annulus fibrosus cells transfected with 80 ng of a vector expressing MSX-1 or MSX-2 or an empty vector (control). H-proline incorporation is indicative of collagen synthesis. Cells transfected with MSX-1 show significantly increased H-proline incorporation than control cells. **, p<0.01

FIG. 3 is a graphical representation showing the level of H-proline incorporation into annulus fibrosus cells transfected with 140 ng of a vector expressing MSX-1 or MSX-2 or an empty vector (control). Cells transfected with MSX-1 or with MSX-2 show significantly increased H-proline incorporation than control cells. *, p<0.05; ***, p<0.001.

FIG. 4 is a graphical representation showing the level of ³⁵S incorporation into extracellular matrix produced by annulus fibrosus cells transfected with 80 ng of a vector expressing MSX-1 or MSX-2 or an empty vector (control). Cells transfected with MSX-1 show significantly increased ³⁵S incorporation than control cells. *, p<0.05; ***, p<0.001.

FIG. 5 is a graphical representation showing the level of ³⁵S incorporation into extracellular matrix produced by annulus fibrosus cells transfected with 140 ng of a vector expressing MSX-1 or MSX-2 or an empty vector (control). Cells transfected with MSX-2 show significantly increased ³⁵S incorporation than control cells. *, p<0.05.

FIG. 6 is a graphical representation showing the level of H-thymidine incorporation (indicative of cell proliferation) in annulus fibrosus cells transfected with 80 ng of a vector expressing MSX-1 or MSX-2 or an empty vector (control). MSX-1 and MSX-2 do not significantly alter H-thymidine incorporation.

FIG. 7 is a graphical representation showing the level of H-thymidine incorporation in annulus fibrosus cells transfected with 140 ng of a vector expressing MSX-1 or MSX-2 or an empty vector (control). MSX-1 and MSX-2 do not significantly alter H-thymidine incorporation.

FIG. 8 shows a schematic, three dimensional view of a device for use in the delivery of a GDF-6 signaling modulator or other composition as described herein to an IVD or a region adjacent or surrounding an IVD in a subject.

FIG. 9 shows a schematic, three dimensional side view of a second embodiment of a device for the delivery of a GDF-6 signaling modulator to a site in a patient's body at which tissue is to be treated.

FIG. 10 shows a schematic, plan view of the device of FIG. 8.

FIG. 11 shows, on an enlarged scale, a distal part of the device encircled by circle ‘A’ in FIG. 10.

FIG. 12 shows a schematic, sectional plan view of a third embodiment of a device for the delivery of a GDF-6 signaling modulator to a site in a patient's body at which tissue is to be treated.

FIG. 13 shows a sectional side view of the device of FIG. 9.

FIG. 14 shows a schematic, sectional plan view of the device of FIG. 9.

FIG. 15 shows a schematic, plan view of a fourth embodiment of a device for the delivery of a GDF-6 signaling modulator to a site in a patient's body at which tissue is to be treated.

FIGS. 16-18 show various stages in the deployment of the device of FIG. 15, in use.

FIG. 19 is a copy of photographic representations showing Western blots of supernatants from Chinese Hamster Ovary (CHO) cells separated by SDS-PAGE and probed with GDF-6 polyclonal antiserum (top panel) or mAb against the FLAG tag (bottom panel). Cells were transfected with expression vectors comprising cDNA encoding full-length GDF-6 (full-length) or an active fragment thereof (active fragment). GDF-6 control (Control) was protein provided with the antibody when purchased. Negative control was mock-transfected cells. Results show bands detected by antiserum recognising GDF-6 in supernatants from cells transfected with cDNA encoding full-length GDF-6 or cDNA encoding the active fragment but not mock transfected cells. The band size corresponds to the commercially provided control protein. The identified GAF-6 bands are also recognised by the FLAG tag-specific mAb (Sigma), but the control protein was not.

FIG. 20 is a copy of a photographic representation showing results of an MRI scan of sheep that have undergone surgery to expose three IVDs, one of which was injured and treated with saline (designated “stab control”), another was injured and treated with recombinant human GDF-6 (designated “GDF-6”), and a third was not injured or treated (designated “un-injured control”). The stab control shown advanced nuclear pulposus degeneration. In contrast, the morphology of the GDF-6 treated disc is more similar to the untreated and undamaged disc than it is to the stab control, indicating that GDF-6 slows and/or prevents IVD degeneration and/or enhances or induces IVD regeneration.

FIG. 21A is a copy of a photographic representation showing results of a Western blot experiment showing enhanced expression of collagen-1 in primary annulus fibrosus cell cultures. Cell cultures were stimulated with GDF-6 (200 ng/mL) or media alone (control) for 7 days then analyzed by Western blot for collagen-1 expression. Data represents expression in 12.5 ug total protein per lane.

FIG. 21B is a copy of a photographic representation showing results of a Western blot experiment showing enhanced expression of collagen-2 in primary annulus fibrosus cell cultures. Cell cultures were stimulated with GDF-6 (200 ng/mL) or media alone (control) for 7 days then analyzed by Western blot for collagen-2 expression. Data represents expression in 12.5 ug total protein per lane.

FIG. 21C is a copy of a photographic representation showing results of a Western blot experiment showing enhanced expression of SOX9 in primary annulus fibrosus cell cultures. Cell cultures were stimulated with GDF-6 (200 ng/mL) or media alone (control) for 7 days then analyzed by Western blot for collagen-1 expression. Data represents expression in 12.5 ug total protein per lane.

FIG. 21D is a copy of a photographic representation showing results of a Western blot experiment showing enhanced expression of collagen-1 in primary nucleus pulposus cell cultures. Cell cultures were stimulated with GDF-6 (200 ng/mL) or media alone (control) for 7 days then analyzed by Western blot for collagen-1 expression. Data represents expression in 12.5 ug total protein per lane.

FIG. 21E is a copy of a photographic representation showing results of a Western blot experiment showing enhanced expression of collagen-2 in primary nucleus pulposus cell cultures. Cell cultures were stimulated with GDF-6 (200 ng/mL) or media alone (control) for 7 days then analyzed by Western blot for collagen-2 expression. Data represents expression in 12.5 ug total protein per lane.

FIG. 21F is a copy of a photographic representation showing results of a Western blot experiment showing enhanced expression of SOX9 in primary nucleus pulposus cell cultures. Cell cultures were stimulated with GDF-6 (200 ng/mL) or media alone (control) for 7 days then analyzed by Western blot for collagen-1 expression. Data represents expression in 12.5 ug total protein per lane.

FIG. 21G is a copy of a photographic representation showing results of a Western blot experiment showing enhanced expression of collagen-1 in primary cultures of cells from IVD endplates. Cell cultures were stimulated with GDF-6 (200 ng/mL) or media alone (control) for 7 days then analyzed by Western blot for collagen-1 expression. Data represents expression in 12.5 ug total protein per lane.

FIG. 21H is a copy of a photographic representation showing results of a Western blot experiment showing enhanced expression of collagen-2 in primary cultures of cells from IVD endplates. Cell cultures were stimulated with GDF-6 (200 ng/mL) or media alone (control) for 7 days then analyzed by Western blot for collagen-2 expression. Data represents expression in 12.5 ug total protein per lane.

FIG. 21I is a copy of a photographic representation showing results of a Western blot experiment showing enhanced expression of SOX9 in primary cultures of cells from IVD endplates. Cell cultures were stimulated with GDF-6 (200 ng/mL) or media alone (control) for 7 days then analyzed by Western blot for collagen-1 expression. Data represents expression in 12.5 ug total protein per lane.

FIG. 22A is a graphical representation showing the level of expression of the chondrogenic marker collagen II at the mRNA level in BM MSC cells and cells differentiated therefrom incubated in the presence of various concentrations of GDF-6 (GDF-6) as indicated on the X-axis. Expression levels were detected using real time quantitative PCR. Relative expression is indicated on the Y-axis.

FIG. 22B is a graphical representation showing the level of expression of the chondrogenic marker Aggrecan at the mRNA level in BM MSC cells and cells differentiated therefrom incubated in the presence of various concentrations of GDF-6 (GDF-6) as indicated on the X-axis. Expression levels were detected using real time quantitative PCR. Relative expression is indicated on the Y-axis.

FIG. 22C is a graphical representation showing the level of expression of the chondrogenic marker Sox9 at the mRNA level in BM MSC cells and cells differentiated therefrom incubated in the presence of various concentrations of GDF-6 (GDF-6) as indicated on the X-axis. Expression levels were detected using real time quantitative PCR. Relative expression is indicated on the Y-axis.

FIG. 23 is a graphical representation showing the level of expression of BMP-2, BMP-17 or GDF-6 (GDF-6) as indicated in bone marrow mesenchymal stem cells (BM MSCs) after 1, 3, 5 and 7 days (as indicated). Results are expressed relative to standard, constant, house-keeping genes GAPDH and HPRT.

FIG. 24 is a graphical representation showing the number of cells in BM MSC cultures incubated in increasing concentrations of GDF-6 (as indicated). Results are expressed as a percentage of control cultures containing no GDF-6 stimulation.

FIG. 25 is a graphical representation showing results of cell migration assays following incubation with or without increasing quantities of GDF-6 (as indicated). Cell counts are expressed as a percentage of negative control wells (containing no GDF-6).

FIG. 26 is a copy of a photographic representation showing results of an MRI scan of sheep that have undergone surgery to expose three IVDs, one of which was injured and treated with saline (designated “stab control”), another was injured and treated with recombinant human GDF-6 (designated “GDF-6”), and a third was not injured or treated (designated “un-injured control”). The stab control shows advanced nuclear pulposus degeneration. In contrast, the morphology of the GDF-6 treated disc is more similar to the untreated and undamaged disc than it is to the stab control, indicating that GDF-6 slows and/or prevents IVD degeneration and/or enhances or induces IVD regeneration.

FIG. 27A is a copy of a photomicrograph showing 100× and 400× images of sections of the endplate of disc-2 from sheep 5497 that has under gone surgery to expose the IVDs. The sections were stained with haematoxylin and eosin and viewed under Olympus light microscope. The images shown are from a disc representing an exposed control without receiving any annular injury (un-injured control).

FIG. 27B is a copy of a photomicrograph showing 100× and 400× images of sections of the endplate of disc-2 from sheep 5497 that has under gone surgery to expose the IVDs. The sections were stained with haematoxylin and eosin and viewed under Olympus light microscope. The images shown are from a disc that received an annular tear with saline injection (stab control).

FIG. 27C is a copy of a photomicrograph showing 100× and 400× images of sections of the endplate of disc-2 from sheep 5497 that has under gone surgery to expose the IVDs. The sections were stained with haematoxylin and eosin and viewed under Olympus light microscope. The images shown are from a disc that received an annular tear and treated with GDF-6.

FIG. 28A is a copy of a photomicrograph showing 40× images of sections of discs from sheep that have under gone surgery to expose the IVDs. The sections were stained with Alcian Blue to visualize proteoglycan and viewed under Olympus light microscope. The images shown are from control discs (control), surgically exposed un-unjured discs (exposed), those that received an annular tear with saline injection (stabbed), and those that received an annular tear and treated with GDF-6 (BMP-13).

FIG. 28B provides copies of graphical representations showing the intensity of Alcian blue stained sheep discal tissues after 4 month injection. Panel (i) shows percentage area of stained discal tissue (n=3±SD) on microscopic examination and quantitation by Imagel Software of control discs (control), surgically exposed un-unjured discs (exposed), discs that received an annular tear with saline injection (stabbed), and those that received an annular tear and treatment with GDF-6 (BMP-13). Panel (ii) shows percentage area of stained discal tissue (n=3±SD) on macroscopic examination and quantitation by Imagel Software of control discs (control), surgically exposed un-unjured discs (exposed), discs that received an annular tear with saline injection (stabbed), and those that received an annular tear and treatment with GDF-6 (BMP-13). Panel (iii) shows percentage area of stained discal tissue on microscopic examination and quantitation by Imagel Software of a single sample (n=1) of a control disc (control), surgically exposed un-unjured disc (exposed), disc that received an annular tear with saline injection (stabbed), and disc that received an annular tear and treatment with GDF-6 (BMP-13).

FIG. 28C is a copy of a photomicrograph showing 40× and 100× images of sections of discs from sheep that have under gone surgery to expose the IVDs. The sections were stained with haematoxylin and counterstained with Eosin to visualize tissue architecture and viewed under Olympus light microscope. The images shown are from control surgically exposed uninjured discs (1), those that received an annular tear with saline injection (2), and those that received an annular tear and treated with GDF-6 (BMP-13; 3).

FIG. 28D is a copy of a photomicrograph showing 40× and 100× images of sections of discs from sheep that have under gone surgery to expose the IVDs. The sections were visualized with polarized light to view collagen deposition. The images shown are from control discs (NV-control), surgically exposed un-unjured discs (Exp-control), those that received an annular tear with saline injection (saline), and those that received an annular tear and treated with GDF-6 (BMP).

FIG. 29A is a copy of a photographic representation showing results of a Western blot experiment showing a dose response of enhanced expression of SOX9 in primary annulus fibrosus (AF) cell cultures. AF cell cultures were stimulated with increasing doses of GDF-6 as shown (200-600 ng/mL) or media alone (control) for 7 days then analyzed by Western blot for SOX9 expression. Data represents expression in 12.5 ug total protein per lane.

FIG. 29B is a copy of a photographic representation showing results of a Western blot experiment showing a dose response of enhanced expression of collagen I and collagen II in primary end-plate cultures (EP) derived from one disc sample-culture 1. EP cell cultures were stimulated with increasing doses of GDF-6 as shown (200-600 ng/mL) or media alone (control) for 7 days then analyzed by Western blot for collagen II and collagen II expression. Data represents expression in 12.5 ug total protein per lane.

FIG. 29C is a copy of a photographic representation showing results of a Western blot experiment showing a dose response of enhanced expression of collagen I and collagen II in primary end-plate cultures (EP) derived from another disc sample-culture 2. EP cell cultures were stimulated with increasing doses of GDF-6 as shown (200-600 ng/mL) or media alone (control) for 7 days then analyzed by Western blot for collagen II and collagen II expression. Data represents expression in 12.5 ug total protein per lane.

FIG. 30A is a graphical representation showing the level of expression of the aggrecan compared to Alkaline phosphatase; and expression of CD166 compared to CD105. BM MSC were cultured with and without GDF-6 (300 ng/mL) over a two week period, cells were harvested, and expression levels were detected using real time quantitative PCR. Relative expression levels is indicated on the Y-axis for each gene shown on the X-axis.

FIG. 30B is a graphical representation showing the level of expression of the SOX9, Runx2, Noggin and Chordin. BM MSC were cultured with and without GDF-6 (300 ng/mL) over a two week period, cells were harvested, and expression levels were detected using real time quantitative PCR. Relative expression levels is indicated on the Y-axis for each gene shown on the X-axis.

FIG. 30C is a graphical representation showing the level of expression BMP2, BMP4, BMP13 and Msx2. BM MSC were cultured with and without GDF-6 (300 ng/mL) over a two week period, cells were harvested, and expression levels were detected using real time quantitative PCR. Relative expression levels is indicated on the Y-axis for each gene shown on the X-axis.

FIG. 30D is a copy of a photomicrograph showing images of Alcian Blue staining of BM MSC cultures treated with control media, osteo-differentiation media, or media with GDF-6 (300 ng/mL), over a two week period and visualized under a light microscope at 40× magnification.

FIG. 30E is a copy of a photomicrograph showing images of Alizarin red staining of BM MSC cultures treated control media, control media +GDF-6 (BMP13), osteo-differentiation media, or osteo-diff media +GDF-6 (BMP13), over a two week period and visualized under a light microscope at 40× magnification. The concentration of GDF-6 was varied as shown.

### DETAILED DESCRIPTION OF THE PREFERRED EMBODIMENTS

1. Modulators of GDF-6 Signaling

A composition as described herein comprises any one or more modulators of GDF-6 signaling in an IVD or cell or tissue thereof in a subject. For example, a modulator enhances GDF-6 signaling in an IVD or cell or tissue thereof in a subject. Such a modulator is also referred to as a GDF-6 signaling enhancer or a GDF-6 signaling agonist.

The present invention contemplates any modulator of GDF-6 signaling. For example, the modulator is a peptide, a polypeptide, a nucleic acid, an antibody, an antibody fragment or a small molecule.

1.1 Polypeptide Modulators

In one example, a modulator of GDF-6 signaling in an IVD or cell or tissue thereof is a peptide or polypeptide. For example, a modulator is a peptide or polypeptide that mediates GDF-6 signaling in an IVD or cell or tissue thereof. For example, a modulator is a polypeptide individually or collectively selected from the group consisting of:
(i) a polypeptide selected from the group consisting of GDF-6, MSX-1, MSX-2, BMPR-1A, BMPR-IB, BMPR-II, Smad-1, Smad-5, Smad-8 and Smad-4;
(ii) an active fragment of (i);
(iii) an analog of (i) or (ii); and
(iv) a derivative of any one of (i) to (iii).

By “individually” is meant that the invention encompasses the recited polypeptides or groups of polypeptides separately, and that, notwithstanding that individual polypeptides or groups of polypeptides may not be separately listed herein the accompanying claims may define such polypeptides or groups of polypeptides separately and divisibly from each other.

By “collectively” is meant that the invention encompasses any number or combination of the recited polypeptides or groups of polypeptides, and that, notwithstanding that such numbers or combinations of polypeptides or groups of peptides may not be specifically listed herein the accompanying claims may define such combinations or sub-combinations separately and divisibly from any other combination of polypeptides or groups of polypeptides.

By “active fragment” is meant a portion of a polypeptide that retains the ability of that polypeptide to modulate GDF-6 signaling. An active fragment may have the same level of activity as the original protein or an enhanced or reduced level of activity compared to the level of activity of the original protein. Methods for determining GDF-6 activity will be apparent to the skilled artisan and/or described herein.

In one preferred example of the invention, the modulator of GDF-6 signaling in an IVD or cell or tissue thereof is a GDF-6 polypeptide or an active fragment thereof. As used herein, the term “GDF-6” shall be taken to mean a polypeptide comprising an amino acid sequence at least about 80% identical to the sequence set forth in SEQ ID NO: 2 or 3 or encoded by a nucleic acid comprising the sequence set forth in SEQ ID NO: 1, wherein said polypeptide is capable of modulating GDF-6 signaling in an IVD or cell or tissue thereof. Such a GDF-6 polypeptide is useful because it binds to a transmembrane receptor and enhances GDF-6 signaling in an IVD or cell or tissue thereof. Accordingly, it is not necessary for the polypeptide to enter a cell to induce GDF-6 signaling.

Preferably, the polypeptide has at least about 90% identity or 95% identity or 98% identity or 99% identity to the sequence set forth in SEQ ID NO: 2 or 3 or encoded by a nucleic acid comprising the sequence set forth in SEQ ID NO: 1, wherein said polypeptide is capable of modulating GDF-6 signaling in an IVD or cell or tissue thereof.

In determining whether or not two sequences fall within these defined percentage identity limits, those skilled in the art will be aware that it is possible to conduct a side-by-side comparison of the sequences. In such comparisons or alignments, differences will arise in the positioning of non-identical residues depending upon the algorithm used to perform the alignment. In the present context, references to percentage 30 identities and similarities between two or more sequences shall be taken to refer to the number of identical and similar residues respectively, between said sequences as determined using any standard algorithm known to those skilled in the art. For example, nucleotide identities and similarities are calculated using software of the Computer Genetics Group, Inc., University Research Park, Madison, Wis., United States of America, e.g., using the GAP program of Devereaux et al., Nucl. Acids Res. 12, 387-395, 1984, which utilizes the algorithm of Needleman and Wunsch, J. Mol. Biol. 48, 443-453, 1970. Alternatively, the CLUSTAL W algorithm of Thompson et al., Nucl. Acids Res. 22, 4673-4680, 1994, is used to obtain an alignment of multiple sequences, wherein it is necessary or desirable to maximize the number of identical/similar residues and to minimize the number and/or length of sequence gaps in the alignment. Sequence alignments can also be performed using a variety of other commercially available sequence analysis programs, such as, for example, the BLAST program available at NCBI.

In a preferred example, an active fragment of GDF-6 is an isolated peptide having GDF-6 signaling activity or an analog or derivative thereof, wherein said peptide consists of the sequence of a C-terminal fragment of a GDF-6 polypeptide or an analog or derivative thereof and optionally comprises an N-terminal methionine residue. In one example, the peptide, analog or derivative does not comprise all of the pro-region of a GDF-6 polypeptide. In another example, the peptide, analog or derivative consists of about 120 amino acids derived from the C-terminus of native GDF-6. In a further example, the peptide, analog or derivative comprises sufficient cysteine residues to form homodimers and/or heterodimers under non-reducing conditions. For example, the peptide comprises a sequence set forth in any one of SEQ ID NOs: 24 or 25 or a sequence having at least about 90% identity to any one of SEQ ID NOs: SEQ ID NO: 24 or 25. In this respect, the sequence set forth in SEQ ID NO: 25 is that of an active fragment of GDF-6 fused at its carboxy-terminus to a FLAG epitope and a TEV protease cleavage site. In one example, the peptide comprises an N-terminal methionine residue.

In another example, the peptide having GDF-6 signaling activity or an analog or derivative thereof is a retro-peptide analog, e.g., comprising a sequence set forth in SEQ ID NO: 34 or 35.

In another example, the isolated peptide having GDF-6 signaling activity or an analog or derivative thereof comprises one or more D-amino acids.

In a further example, the isolated peptide having GDF-6 signaling activity or an analog or derivative thereof is a retro-inverted analog, e.g., comprising a sequence set forth in SEQ ID NO: 36 or 37.

As used herein, the term “consisting essentially of” shall be taken to mean that the active fragment comprises the recited sequence and any other unstated features that do not materially affect the GDF-6 signaling modulatory properties of the active fragment.

The term “consisting of” means that the active fragment only has the recited sequence.

In one example, a GDF-6 polypeptide or active fragment thereof or analog or derivative thereof comprises a pair of subunits disulfide bonded to produce a dimer. In this respect, the dimer can contain two GDF-6 polypeptides or two active fragments or two analogs or two derivatives, or mixtures of a GDF-6 polypeptide and/or active fragment and/or analog and/or derivative. For example, the dimer comprises a GDF-6 polypeptide and an active fragment or a GDF-6 polypeptide and an analog and/or an active fragment and an analog or a GDF-6 polypeptide and a derivative or an active fragment and a derivative or an analog and a derivative.

In another example, the polypeptide is a MSX-1 polypeptide or an active fragment thereof. As used herein, the term “MSX-1” shall be taken to mean a polypeptide comprising an amino acid sequence at least about 80% identical to the sequence set forth in SEQ ID NO: 5 or encoded by a nucleic acid comprising the sequence set forth in SEQ ID NO: 4, wherein said polypeptide is capable of modulating GDF-6 signaling in an IVD or cell or tissue thereof.

Preferably, the polypeptide has at least about 90% identity or 95% identity or 98% identity or 99% identity to the sequence set forth in SEQ ID NO: 5 or encoded by a nucleic acid comprising the sequence set forth in SEQ ID NO: 4, wherein said polypeptide is capable of modulating GDF-6 signaling in an IVD or cell or tissue thereof.

In one example, a MSX1 polypeptide or active fragment thereof or analog or derivative thereof comprises a pair of MSX1 subunits bound to one another to produce a dimer. In this respect, the dimer can contain two MSX1 polypeptides or two active fragments or two analogs or two derivatives, or mixtures of a MSX1 polypeptide and/or active fragment and/or analog and/or derivative. For example, the dimer comprises a MSX1 polypeptide and an active fragment or a MSX1 polypeptide and an analog and/or an active fragment and an analog or a MSX1 polypeptide and a derivative or an active fragment and a derivative or an analog and a derivative.

In another example, the MSX1 polypeptide or active fragment thereof or analog or derivative thereof is dimerized with a Dlx1 protein, e.g., as described in Zhang et al., Mol. and Cell. Biol. 17: 2920-2932, 1997.

In another example, the polypeptide is a MSX-2 polypeptide or an active fragment thereof. As used herein, the term “MSX-2” shall be taken to mean a polypeptide comprising an amino acid sequence at least about 80% identical to the sequence set forth in SEQ ID NO: 7 or encoded by a nucleic acid comprising the sequence set forth in SEQ ID NO: 6, wherein said polypeptide is capable of modulating GDF-6 signaling in an IVD or cell or tissue thereof.

Preferably, the polypeptide has at least about 90% identity or 95% identity or 98% identity or 99% identity to the sequence set forth in SEQ ID NO: 7 or encoded by a nucleic acid comprising the sequence set forth in SEQ ID NO: 6, wherein said polypeptide is capable of modulating GDF-6 signaling in an IVD or cell or tissue thereof.

The sequence of additional peptide or polypeptide modulators of GDF-6 signaling are readily derivable from publicly available databases, such as, for example, the Genbank database available from NCBI. Moreover, methods for determining a peptide or polypeptide having GDF-6 modulatory activity will be apparent tot eh skilled artisan, e.g., based on the description herein.

The present invention also clearly extends to variants of a GDF-6 modulatory peptide or polypeptide described herein, such as derivatives and/or analogs, by modification to the sequences provided herein. The invention also extends to homologs i.e., functionally-equivalent peptides or polypeptide having related sequences to the sequences provided herein.

It is understood by the skilled artisan that, inherent in the definition of a biologically functional equivalent protein or peptide, is the concept that there is a limit to the number of changes that may be made within a defined portion of the molecule and still result in a molecule with an acceptable level of equivalent biological activity. Biologically functional equivalent peptides are thus defined herein as those peptides in which specific amino acids may be substituted or deleted. Particular embodiments encompass variants that have one, two, three, four, five or more variations in the amino acid sequence relative to a base peptide subject to the retention of an ability to modulate GDF-6 signaling and, preferably reduce or prevent or delay IVD degeneration and/or enhance or induce IVD regeneration. Of course, a plurality of variants may be made and used in accordance with the invention.

A modulator of GDF-6 signaling, e.g., a GDF-6 polypeptide or functional fragment thereof may also be glycosylated. Glycosylation is the modification of a protein by addition of one or more oligosaccharide groups. There are usually two types of glycosylation: O-linked oligosaccharides are attached to serine or threonine residues while N-linked oligosaccharides are attached to asparagine residues when they are part of the sequence Asn-X-Ser/Thr, where X can be any amino acid except proline. Glycosylation can dramatically affect the physical properties of proteins and can also be important in protein stability, secretion, half-life, and subcellular localization. In some embodiments, the modulator of GDF-6 signaling comprise N-linked oligosaccharaides. In other embodiments, the modulator of GDF-6 signaling comprise O-linked oligosaccharides. In yet other embodiments, the modulator of GDF-6 signaling of this inventions comprise both N-linked and O-linked oligosaccharides. In some embodiments, the glycosylation pattern of the modulator of GDF-6 signaling may be modified to control the carbohydrate composition of the glycoprotein.

Based on the definition of “modulator of GDF-6 signaling” herein above, the skilled artisan will be aware that a bone morphogenetic protein (BMP)-2, BMP-4, BMP-7 (syn. osteogenic protein (OP)-1) and/or BMP-14 is not a modulator of GDF-6 signaling. Accordingly, the term “modulator of GDF-6 signaling” does not encompass BMP-2, BMP-4, BMP-7/OP-1 or BMP-14.

Peptide and Polypeptide Derivatives

As used herein the term “derivative” shall be taken to mean a peptide or polypeptide that is derived from a peptide or polypeptide modulator of GDF-6 signaling as described herein, e.g., a fragment or processed form of the peptide or polypeptide, or a molecule comprising one or more amino acid substitutions, or comprising additional amino acid residues or non-amino acid substituents, relative to the base peptide or polypeptide from which it is derived. The term “derivative” also encompasses fusion proteins comprising a peptide of the invention.

Exemplary fusion protein comprises a label, such as, for example, an epitope, e.g., a FLAG epitope or a V5 epitope or an HA epitope. Such a tag is useful for, for example, purifying the fusion protein. Preferably, the label is a FLAG epitope.

A “conservative amino acid substitution” is one in which an amino acid residue is replaced with another amino acid residue without disturbing the overall structure of the peptide. Such changes tend to rely on similarity in hydrophilicity and/or polarity of the substituent. The size and/or charge of the side chains also are relevant factors in determining which substitutions are conservative. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine).

Those skilled in the art are well aware that the following substitutions are permissible conservative substitutions (i) substitutions involving arginine, lysine and histidine; (ii) substitutions involving alanine, glycine and serine; and (iii) substitutions involving phenylalanine, tryptophan and tyrosine.

The importance of the hydropathic amino acid index in conferring interactive biological function on a protein is generally understood in the art (Kyte & Doolittle, J. Mol. Biol. 157, 105-132, 1982). It is known that certain amino acids may be substituted for other amino acids having a similar hydropathic index or score and still retain a similar biological activity. The hydropathic index of amino acids also may be considered in determining a conservative substitution that produces a functionally equivalent molecule. Each amino acid has been assigned a hydropathic index on the basis of their hydrophobicity and charge characteristics, as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making changes based upon the hydropathic index, the substitution of amino acids whose hydropathic indices are within +/−0.2 is preferred. More preferably, the substitution will involve amino acids having hydropathic indices within +/−0.1, and more preferably within about +/−0.05.

Non-amino acid substituents may be linked covalently to a peptide e.g., via an amino terminal amino acid residue, a carboxy terminal amino acid residue, or at an internal amino acid residue. Such modifications include the addition of a protective or capping group on a reactive moiety in the peptide, addition of a detectable label, and other changes that do not adversely destroy the activity of the peptide compound. For example, particular amino acid residues may be derivatized or chemically modified in order to enhance the stability of the peptide or to permit coupling of the peptide to other agents, particularly lipids.

Chemical moieties may be linked covalently to a peptide or polypeptide e.g., via an amino terminal amino acid residue, a carboxy terminal amino acid residue, or at an internal amino acid residue. Such modifications include the addition of a protective or capping group on a reactive moiety in the peptide, addition of a detectable label, and other changes that do not adversely destroy the activity of the peptide compound.

An “amino terminal capping group” of a peptide or polypeptide described herein is any chemical compound or moiety that is covalently linked or conjugated to the amino terminal amino acid residue of a peptide compound. An amino terminal capping group may be useful to inhibit or prevent intramolecular cyclization or intermolecular polymerization, to protect the amino terminus from an undesirable reaction with other molecules, to provide additional antioxidative activity, or to provide a combination of these properties. A peptide or polypeptide that possesses an amino terminal capping group may possess other beneficial activities as compared with the uncapped peptide, such as enhanced efficacy or reduced side effects. Examples of amino terminal capping groups that are useful in preparing a peptide or polypeptide include, but are not limited to, 1 to 6 naturally occurring L-amino acid residues, preferably, 1-6 lysine residues, 1-6 arginine residues, or a combination of lysine and arginine residues; urethanes; urea compounds; lipoic acid (“Lip”); glucose-3-O-glycolic acid moiety (“Gga”); or an acyl group that is covalently linked to the amino terminal amino acid residue of a peptide, wherein such acyl groups useful in the compositions of the invention may have a carbonyl group and a hydrocarbon chain that ranges from one carbon atom (e.g., as in an acetyl moiety) to up to 25 carbons (e.g., palmitoyl group, “Palm” (16:0) and docosahexaenoyl group, “DHA” (C22:6-3)). Furthermore, the carbon chain of the acyl group may be saturated, as in Palm, or unsaturated, as in DHA. It is understood that when an acid, such as docosahexaenoic acid, palmitic acid, or lipoic acid is designated as an amino terminal capping group, the resultant peptide compound is the condensed product of the uncapped peptide and the acid.

A “carboxy terminal capping group” of a peptide or polypeptide is any chemical compound or moiety that is covalently linked or conjugated to the carboxy terminal amino acid residue of the peptide or polypeptide. A peptide or polypeptide possessing a carboxy terminal capping group may also possess other beneficial activities as compared with the uncapped peptide, such as enhanced efficacy, reduced side effects, enhanced hydrophilicity, enhanced hydrophobicity. Carboxy terminal capping groups that are particularly useful include primary or secondary amines that are linked by an amide bond to the α-carboxyl group of the carboxy terminal amino acid of the peptide or polypeptide. Other carboxy-terminal capping groups useful in the invention include aliphatic primary and secondary alcohols and aromatic phenolic derivatives, including flavenoids, with 1 to 26 carbon atoms, which form esters when linked to the carboxylic acid group of the carboxy terminal amino acid residue of a peptide or polypeptide described herein.

Other chemical modifications of a peptide or polypeptide, include, for example, glycosylation, acetylation (including N-terminal acetylation), carboxylation, carbonylation, phosphorylation, PEGylation, amidation, addition of trans olefin, substitution of α-hydrogens with methyl groups, derivatization by known protecting/blocking groups, circularization, inhibition of proteolytic cleavage (e.g., using D amino acids), linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH₄, acetylation, formylation, oxidation, reduction, etc.

Peptide Analogs

In another example of the invention, a peptide or polypeptide analog having GDF-6 signaling modulatory activity is prepared. As used herein, the term “analog” shall be taken to mean a peptide or polypeptide that is modified to comprise one or more non-naturally-occurring amino acids.

Analogs may also comprise sterically similar compounds that mimic critical subdomains of a peptide or polypeptide. Such “peptidomimetics” are produced by modeling and chemical design processes known to those of skill in the art.

Preferred analogs of a GDF-6 signaling modulatory peptides or polypeptides comprise one or more non-naturally occurring amino acids or amino acid analogs. For example, a peptide or polypeptide modulator comprises one or more naturally occurring non-genetically encoded L-amino acids, synthetic L-amino acids or D-enantiomers of an amino acid. For example, the peptide comprises only D-amino acids. For example, the analog comprises one or more residues selected from the group consisting of: hydroxyproline, β-alanine, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylananine 3-benzothienyl alanine 4-chlorophenylalanine, 2-fluorophenylalanine, 3-fluorophenylalanine, 4-fluorophenylalanine, penicillamine, 1,2,3,4-tetrahydro-tic isoquinoline-3-carboxylic acid β-2-thienylalanine, methionine sulfoxide, homoarginine, N-acetyl lysine, 2,4-diamino butyric acid, p-aminophenylalanine, N-methylvaline, homocysteine, homoserine, ε-amino hexanoic acid, δ-amino valeric acid, 2,3-diaminobutyric acid and mixtures thereof.

Other amino acid residues that are useful for making the peptides or polypeptides or analogs thereof can be found, e.g., in Fasman, 1989, CRC Practical Handbook of Biochemistry and Molecular Biology, CRC Press, Inc., and the references cited therein.

The present invention additionally encompasses an isostere of a peptide or polypeptide described herein. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) known to those skilled in the art. Such modifications include modifications of the amide nitrogen, the α-carbon, amide carbonyl, complete replacement of the amide bond, extensions, deletions or backbone crosslinks. Several peptide backbone modifications are known, including ψ[CH₂S], ψ[CH₂NH], ψ[CSNH₂], ψ[NHCO], ψ[COCH₂], and ψ[(E) or (Z) CH═CH]. In the nomenclature used above, ψ indicates the absence of an amide bond. The structure that replaces the amide group is specified within the brackets.

Other modifications include, for example, an N-alkyl (or aryl) substitution (ψ [CONR]), or backbone crosslinking to construct lactams and other cyclic structures. Other derivatives of the modulator compounds of the invention include C-terminal hydroxymethyl derivatives, O-modified derivatives (e.g., C-terminal hydroxymethyl benzyl ether), N-terminally modified derivatives including substituted amides such as alkylamides and hydrazides.

In another example, a peptide analog is a retro peptide or polypeptide (see, for example, Goodman et al., Accounts of Chemical Research, 12:1-7, 1979). A retro peptide or polypeptide comprises a reversed amino acid sequence of a peptide modulator described herein. For example, the retro-peptide comprises a sequence set forth in SEQ ID NO: 34 or 35.

In a further example, an analog of a peptide described herein is a retro-inverso peptide or polypeptide (Sela and Zisman, FASEB J. 11:449, 1997). Evolution has ensured the almost exclusive occurrence of L-amino acids in naturally occurring proteins. As a consequence, virtually all proteases cleave peptide bonds between adjacent L-amino acids. Accordingly, artificial proteins or peptides composed of D-amino acids are preferably resistant to proteolytic breakdown. Retro-inverso peptide or polypeptide analogs are isomers of linear peptides in which the direction of the amino acid sequence is reversed (retro) and the chirality, D- or L-, of one or more amino acids therein is inverted (inverso) e.g., using D-amino acids rather than L-amino acids, e.g., Jameson et al., Nature, 368, 744-746 (1994); Brady et al., Nature, 368, 692-693 (1994). The net result of combining D-enantiomers and reverse synthesis is that the positions of carbonyl and amino groups in each amide bond are exchanged, while the position of the side-chain groups at each alpha carbon is preserved. An advantage of retro-inverso peptides is their enhanced activity in vivo due to improved resistance to proteolytic degradation, i.e., the peptide has enhanced stability. (e.g., Chorev et al., Trends Biotech. 13, 438-445, 1995).

Retro-inverso or retroinverso peptide or polypeptide analogs may be complete or partial. Complete retro-inverso peptides or polypeptides are those in which a complete sequence of a peptide described herein is reversed and the chirality of each amino acid other than glycine in a sequence is inverted. The exclusion of glycine is based on the fact that glycine does not have a chiral analog. Partial retro-inverso peptide or polypeptide analogs are those in which only some of the peptide bonds are reversed and the chirality of only those amino acid residues in the reversed portion is inverted. In one example, a retro-inverso peptide analog comprises a sequence set forth in SEQ ID NO: 36 or 37.

Protein Transduction Domains

Some peptides or polypeptides must enter a cell to exert their biological activity. To facilitate peptide entry into a cell, the peptide or polypeptide may be conjugated to (e.g., expressed as a fusion with) a protein transduction domain. As used herein, the term “protein transduction domain” shall be taken to mean a peptide or protein that is capable of enhancing, increasing or assisting penetration or uptake of a compound conjugated to the protein transduction domain into a cell either in vitro or in vivo. Those skilled in the art will be aware that synthetic or recombinant peptides can be delivered into cells through association with a protein transduction domain such as the TAT sequence from HIV or the Penetratin sequence from the Antennapaedia homeodomain protein (see, for example, Temsamani and Vidal, Drug Discovery Today 9: 1012-1019, 2004, for review).

A suitable protein transduction domain will be apparent to the skilled artisan and includes, for example, HIV-1 TAT basic region (e.g., SEQ ID NO: 8) or polyarginine (e.g., SEQ ID NO: 9).

For example, a HIV-1 TAT basic region has been shown to be capable of delivering a polypeptide into an IVD cell, e.g., US Patent Publication No. 20040197867.

Additional suitable protein transduction domains are described, for example, by Zhao and Weisledder Medicinal Research Reviews, 24: 1-12, 2004; or by Wagstaff and Jans, Current Medicinal Chemistry, 13: 1371-1387, 2006; or in US Patent Publication No. 20040197867.

Linkers

A peptide or polypeptide modulator of GDF-6 signaling may be linked to another peptidyl moiety (e.g., for immunodetection such as a FLAG epitope, or for targeting such as a protein transduction domain), albeit separated there from by a linker .

Preferred linkers facilitate the independent folding of each peptidyl moiety in the assembled peptide or polypeptide, thereby reducing steric hindrance of one moiety by another moiety. The amino acid composition of a linker peptide is important for stability and folding of a fusion protein, rather than a specific sequence (Robinson and Sauer Proc. Natl. Acad. Sci. 95: 5929-5934, 1998).

Suitable linkers will be apparent to the skilled artisan and are predominantly hydrophilic, i.e. the residues in the linker are hydrophilic.

It is also often unfavorable to utilize a linker sequence having a high propensity to adopt α-helix or β-strand structures, which could limit the flexibility of the peptidyl moieties and reduce functionality. Accordingly, preferred linkers may have a preference to adopt extended conformations.

Preferred linkers comprise a high content of glycine and/or serine residues. Linkers comprising glycine and/or serine have a high freedom degree for linking of two proteins, i.e., they enable the fused proteins to fold and produce functional proteins.

Glycine-rich linkers are particularly preferred because they force the linker to adopt a loop conformation. The absence of a β-carbon from glycine also permits the polypeptide backbone to access dihedral angles that are energetically forbidden for other amino acids. A particularly preferred linker in the present context consists of polyglycine i.e., between about 2 and 6 glycine residues, or a single glycine residue.

Chemical Synthesis of Peptides, Polypeptides and Analogs Thereof

GDF-6 modulatory peptides or polypeptides and any derivatives, analogs or homologs thereof are readily synthesized from their determined amino acid sequences using standard techniques, e.g., using BOC or FMOC chemistry. Synthetic peptides and polypeptides are prepared using known techniques of solid phase, liquid phase, or peptide condensation, or any combination thereof, and can include natural and/or unnatural amino acids. Amino acids used for peptide synthesis may be standard Boc (Nα-amino protected Nα-t-butyloxycarbonyl) amino acid resin with the deprotecting, neutralization, coupling and wash protocols of the original solid phase procedure of Merrifield, J. Am. Chem. Soc., 85:2149-2154, 1963, or the base-labile Nα-amino protected 9-fluorenylmethoxycarbonyl (Fmoc) amino acids described by Carpino and Han, J. Org. Chem., 37:3403-3409, 1972. Both Fmoc and Boc Nα-amino protected amino acids can be obtained from various commercial sources, such as, for example, Fluka, Bachem, Advanced Chemtech, Sigma, Cambridge Research Biochemical, Bachem, or Peninsula Labs.

The Merrifield method of synthesis (Merrifield, J Am Chem Soc, 85,:2149-2154, 1963) and the myriad of available improvements on that technology are described in the art (see e.g., Synthetic Peptides: A User's Guide, Grant, ed. (1992) W.H. Freeman & Co., New York, pp. 382; Jones (1994) The Chemical Synthesis of Peptides, Clarendon Press, Oxford, pp. 230.); Barany, G. and Merrifield, R. B. (1979) in The Peptides (Gross, E. and Meienhofer, J. eds.), vol. 2, pp. 1-284, Academic Press, New York; Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474.

Generally, chemical synthesis methods comprise the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B.Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis. Synthesis. Biology, Vol.1, for classical solution synthesis. These methods are suitable for synthesis of a peptide of the present invention or an analog or derivative thereof.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene- chloromethylstyrene copolymers and divinylbenzene-benzhydrylaminopolystyrene copolymers.

A peptide, polypeptide, analog or derivative as described herein can also be chemically prepared by other methods such as by the method of simultaneous multiple peptide synthesis. See, e.g., Houghten Proc. Natl. Acad. Sci. USA 82: 5131-5135, 1985 or U.S. Pat. No. 4,631, 211.

Synthetic peptides may also be produced using techniques known in the art and described, for example, in Stewart and Young (In: Solid Phase Synthesis, Second Edition, Pierce Chemical Co., Rockford, Ill. (1984) and/or Fields and Noble (Int. J. Pept. Protein Res., 35:161-214, 1990), or using automated synthesizers.

Recombinant Peptide Production

Alternatively, or in addition, a peptide or polypeptide or analogue or derivative thereof or fusion protein comprising same is produced as a recombinant protein. To facilitate the production of a recombinant peptide or fusion protein nucleic acid encoding same is preferably isolated or synthesized. Typically the nucleic acid encoding the recombinant protein is/are isolated using a known method, such as, for example, amplification (e.g., using PCR or splice overlap extension) or isolated from nucleic acid from an organism using one or more restriction enzymes or isolated from a library of nucleic acids. Methods for such isolation will be apparent to the ordinary skilled artisan and/or described in Ausubel et al (In: Current Protocols in Molecular Biology. Wiley Interscience, ISBN 047 150338, 1987), Sambrook et al (In: Molecular Cloning: Molecular Cloning: A Laboratory Manual, Cold Spring Harbor Laboratories, New York, Third Edition 2001).

For expressing protein by recombinant means, a protein-encoding nucleic acid is placed in operable connection with a promoter or other regulatory sequence capable of regulating expression in a cell-free system or cellular system. For example, nucleic acid comprising a sequence that encodes a peptide is placed in operable connection with a suitable promoter and maintained in a suitable cell for a time and under conditions sufficient for expression to occur. Nucleic acid encoding a peptide or polypeptide modulator of GDF-6 signaling is described herein or is derived from the publicly available amino acid sequence or the publicly available nucleotide sequence.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Preferred promoters can contain additional copies of one or more specific regulatory elements to further enhance expression and/or alter the spatial expression and/or temporal expression of said nucleic acid.

As used herein, the term “in operable connection with”, “in connection with” or “operably linked to” means positioning a promoter relative to a nucleic acid such that expression of the nucleic acid is controlled by the promoter. For example, a promoter is generally positioned 5′ (upstream) to the nucleic acid, the expression of which it controls. To construct heterologous promoter/nucleic acid combinations (e.g., promoter/nucleic acid encoding a polypeptide), it is generally preferred to position the promoter at a distance from the gene transcription start site that is approximately the same as the distance between that promoter and the nucleic acid it controls in its natural setting, i.e., the gene from which the promoter is derived. As is known in the art, some variation in this distance can be accommodated without loss of promoter function. Should it be preferred that a peptide or polypeptide of the invention is expressed in vitro a suitable promoter includes, but is not limited to a T3 or a T7 bacteriophage promoter (Hanes and Plückthun Proc. Natl. Acad. Sci. USA, 94 4937-4942 1997).

Typical expression vectors for in vitro expression or cell-free expression have been described and include, but are not limited to the TNT T7 and TNT T3 systems (Promega), the pEXP1-DEST and pEXP2-DEST vectors (Invitrogen).

Typical promoters suitable for expression in bacterial cells include, but are not limited to, the lacz promoter, the Ipp promoter, temperature-sensitive XL or XR promoters, T7 promoter, T3 promoter, SP6 promoter or semi-artificial promoters such as the IPTG-inducible tac promoter or lacUV5 promoter. A number of other gene construct systems for expressing the nucleic acid fragment of the invention in bacterial cells are well-known in the art and are described for example, in Ausubel et al (In: Current Protocols in Molecular Biology. Wiley Interscience, ISBN 047 150338, 1987), U.S. Pat. No. 5,763,239 (Diversa Corporation) and Sambrook et al (In: Molecular Cloning: Molecular Cloning: A Laboratory Manual, Cold Spring Harbor Laboratories, New York, Third Edition 2001).

Numerous expression vectors for expression of recombinant polypeptides in bacterial cells and efficient ribosome binding sites have been described, and include, for example, PKC30 (Shimatake and Rosenberg, Nature 292, 128, 1981); pKK173-3 (Amann and Brosius, Gene 40, 183, 1985), pET-3 (Studier and Moffat, J. Mol. Biol. 189, 113, 1986); the pCR vector suite (Invitrogen), pGEM-T Easy vectors (Promega), the pL expression vector suite (Invitrogen) the pBAD/TOPO or pBAD/thio—TOPO series of vectors containing an arabinose-inducible promoter (Invitrogen, Carlsbad, Calif.), the latter of which is designed to also produce fusion proteins with a Trx loop for conformational constraint of the expressed protein; the pFLEX series of expression vectors (Pfizer Inc., CT, USA); the pQE series of expression vectors (QIAGEN, CA, 30 USA), or the pL series of expression vectors (Invitrogen), amongst others.

Typical promoters suitable for expression in eukaryotic cells include the SV40 late promoter, SV40 early promoter and cytomegalovirus (CMV) promoter, CMV IE (cytomegalovirus immediate early) promoter amongst others. Preferred vectors for expression in mammalian cells (e.g., 293, COS, CHO, 10T cells, 293T cells) include, but are not limited to, the pcDNA vector suite supplied by Invitrogen, in particular pcDNA 3.1 myc-His-tag comprising the CMV promoter and encoding a C-terminal 6×His and MYC tag; and the retrovirus vector pSRatkneo (Muller et al., Mol. Cell. Biol., 11, 1785, 1991).

A wide range of additional host/vector systems suitable for expressing a peptide or fusion protein of the present invention are available publicly, and described, for example, in Sambrook et al (In: Molecular cloning, A laboratory manual, second edition, Cold Spring Harbor Laboratory, Cold Spring Harbor, N.Y., 1989).

Means for introducing the isolated nucleic acid molecule or a gene construct comprising same into a cell for expression are well-known to those skilled in the art. The technique used for a given organism depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The modulatory proteins contemplated herein can be expressed from intact or truncated cDNA or from synthetic DNAs in prokaryotic or eukaryotic host cells, and purified, cleaved, refolded, and dimerized to form morphogenically active compositions. Currently preferred host cells include, without limitation, prokaryotic cells such as those from a strain of E. coli, or eukaryotic cells such as those of yeast or mammals. Preferred mammalian cells include CHO cells, COS cells or BSC cells. The skilled artisan will appreciate that other host cells can be used to advantage.

Genes encoding the recombinant modulatory proteins may be expressed in mammalian cell lines such as CHO cells (Chinese Hamster Ovary), COS cells, BHK cells, Balb/c 3T3 cells, 293 cells, or similar cell lines known in the art. Mammalian cells may be grown in any suitable medium, such as α-MEM, Dulbecco's MEM, RPMI 1640, and other media (Freshney, R. I., Culture of Animal Cells. A Manual of Basic Technique. Alan R. Liss, Inc., New York (1983)). The cells may be grown in the presence or absence of a serum supplement such as fetal bovine serum (FBS). The cells may be grown in monolayer or suspension culture, and additionally may be grown in large production scale batches. The expressed modulatory proteins are recovered from the culture medium and can be purified using known methods.

Transformed CHO cells are particularly preferred. CHO cell growth medium may be supplemented with FBS to improve the growth of transformed CHO cells in culture. If it is desired to add FBS, concentrations of FBS as low as 0.5% (v/v) may be added. However, addition of animal-origin proteins always presents the risk of harboring viruses and other deleterious agents. The addition of FBS is not necessary for the practice of the present invention. Dextran sulfate may also be used e.g., a dextran sulfate of molecular weight 500,000 and sulfur content 17% (Pharmacia) or a dextran sulfate of molecular weight 5,000 and sulfur content 18% (Sigma). For example, the growth medium may supplemented with dextran sulfate at a range of concentrations from about 1 to about 500 μg/mL. Higher concentrations of dextran sulfate work but may interfere with cell growth or protein purification. Preferably, the growth medium is supplemented with about 5 μg/ml to about 50 μg/ml dextran sulfate. Most preferably, the growth medium is supplemented with about 10 to 20 μg/ml dextran sulfate.

The yield of recombinant modulatory protein from mammalian cells which express the gene encoding the protein may be measured by known methods such as radioactively labeling cells with [³⁵S]methionine and analyzing secreted proteins by polyacrylamide gel electrophoresis (PAGE) and autoradiography. An amount of functional protein secreted into medium is preferably quantitated by bioassay. Any appropriate bioassay may be used, for example, assay of induction of alkaline phosphatase activity in cells e.g., morphogen-responsive cells, or by assay of chondrogenesis in a mammal such as rat, rabbit, cat or dog.

For recombinant production of active proteins which are normally found in dimeric proteins, such as the BMPs, it is desirable to be able to predictably and consistently produce high amounts of covalently-bonded dimeric protein, which is relatively stable, and to reduce the amount of other isoforms of protein, such as monomer, non-covalently bonded dissociable dimer, and multimeric protein, which are less stable and tend to interconvert when present in the cell culture medium. This may be achieved by including an amount of one or more additional components in culture media that modulate the ratio of cystine to cysteine e.g., L-cystine or L-glutamate. For example, L-cystine may enhance the amount of dimmer produced in culture. Alterntively, L-glutamate may enhance the amount of dimmer produced in culture.

1.2 Nucleic Acid Modulators

In another example, a modulator is a nucleic acid. For example, the modulator is a nucleic acid that encodes a polypeptide modulator as described herein above.

In one example, a nucleic acid modulator encodes a polypeptide selected from the group consisting of:

(i) a nucleic acid encoding a polypeptide selected from the group consisting of GDF-6, MSX-1, MSX-2, BMPR-1A, BMPR-IB, BMPR-II, Smad-1, Smad-5, Smad-8 and Smad-4; and

(ii) a nucleic acid encoding an active fragment of a polypeptide selected from the group consisting of GDF-6, MSX-1, MSX-2, BMPR-1A, BMPR-IB, BMPR-II, Smad-1, Smad-5, Smad-8 and Smad-4.

In one preferred example of the invention, a modulator of GDF-6 signaling in an IVD or cell or tissue thereof is a nucleic acid encoding a GDF-6 polypeptide or an active fragment thereof. For example, the nucleic acid comprises a sequence at least about 80% identical to the sequence set forth in SEQ ID NO: 1, wherein said nucleic acid encodes a polypeptide capable of modulating GDF-6 signaling in an IVD or cell or tissue thereof. Preferably, the nucleic acid has at least about 90% identity or 95% identity or 98% identity or 99% identity to the sequence set forth in SEQ ID NO: 1, wherein said nucleic acid encodes a polypeptide capable of modulating GDF-6 signaling in an IVD or cell or tissue thereof.

In another example, a modulator of GDF-6 signaling in an IVD or cell or tissue thereof is a nucleic acid that encodes a MSX-1 polypeptide or an active fragment thereof. For example, a sequence at least about 80% identical to the sequence set forth in SEQ ID NO: 4, wherein said nucleic acid encodes a polypeptide capable of modulating GDF-6 signaling in an IVD or cell or tissue thereof. Preferably, the nucleic acid has at least about 90% identity or 95% identity or 98% identity or 99% identity to the sequence set forth in SEQ ID NO: 4, wherein said nucleic acid encodes a polypeptide capable of modulating GDF-6 signaling in an IVD or cell or tissue thereof.

In another example, a modulator of GDF-6 signaling in an IVD or cell or tissue thereof is a nucleic acid that encodes a MSX-2 polypeptide or an active fragment thereof. For example, the nucleic acid comprises a sequence at least about 80% identical to the sequence set forth in SEQ ID NO: 6, wherein said nucleic acid encodes a polypeptide capable of modulating GDF-6 signaling in an IVD or cell or tissue thereof. Preferably, the nucleic acid has at least about 90% identity or 95% identity or 98% identity or 99% identity to the sequence set forth in SEQ ID NO: 6, wherein said nucleic acid encodes a polypeptide capable of modulating GDF-6 signaling in an IVD or cell or tissue thereof.

The nucleotide sequence of additional nucleic acids capable of encoding a peptide or polypeptide modulator of GDF-6 signaling are readily derivable from publicly available databases, such as, for example, the Genbank database available from NCBI. Moreover, methods for determining a peptide or polypeptide having GDF-6 modulatory activity will be apparent tot eh skilled artisan, e.g., based on the description herein.

For example, the nucleic acid modulator is a nucleic acid encoding a polypeptide modulator described herein above operably-linked to a promoter for inducing expression in an IVD or a cell or tissue thereof. For example, the nucleic acid is linked to a promoter operable in a variety of cells of a subject, such as, for example, a viral promoter, e.g., a CMV promoter (e.g., a CMV-IE promoter) or a SV-40 promoter. The nucleic acid may also be linked to a promoter that expresses a nucleic acid in an IVD or cell or tissue thereof in nature, such as, for example, a collagen promoter or a matrix metalloproteinase promoter. Additional suitable promoters are described herein and shall be taken to apply mutatis mutandis to the present example of the invention.

Preferably, the nucleic acid modulator of GDF-6 signaling in an IVD or cell or tissue thereof is provided in the form of an expression construct. As used herein, the term “expression construct” refers to a nucleic acid that has the ability to confer expression on a nucleic acid (e.g. a reporter gene and/or a counter-selectable reporter gene) to which it is operably connected, in a cell. Within the context of the present invention, it is to be understood that an expression construct may comprise or be a plasmid, bacteriophage, phagemid, cosmid, virus sub-genomic or genomic fragment, or other nucleic acid capable of maintaining and/or replicating heterologous DNA in an expressible format.

Methods for the construction of a suitable expression construct for performance of the invention will be apparent to the skilled artisan and are described, for example, in Ausubel et al (In: Current Protocols in Molecular Biology. Wiley Interscience, ISBN 047 150338, 1987) or Sambrook et al (In: Molecular Cloning: Molecular Cloning: A Laboratory Manual, Cold Spring Harbor Laboratories, New York, Third Edition 2001).

For example, each of the components of the expression construct is amplified from a suitable template nucleic acid using, for example, PCR and subsequently cloned into a suitable expression construct, such as for example, a plasmid or a phagemid. Alternatively, the nucleic acid required for the assay is, for example, excised from a suitable source, for example, using a restriction endonuclease and cloned into a suitable expression construct.

Vectors suitable for such an expression construct are known in the art and/or described herein. For example, an expression vector suitable for the method of the present invention in a mammalian cell is, for example, a vector of the pcDNA vector suite supplied by Invitrogen, a vector of the pCI vector suite (Promega), a vector of the pCMV vector suite (Clontech), a pM vector (Clontech), a pSI vector (Promega), a VP 16 vector (Clontech) or a vector of the pcDNA vector suite (Invitrogen).

The skilled artisan will be aware of additional vectors and sources of such vectors, such as, for example, Invitrogen Corporation, Clontech or Promega.

Alternatively, an expression construct of the invention is a viral vector. Suitable viral vectors are known in the art and commercially available. Conventional viral-based systems for the delivery of a nucleic acid and integration of that, nucleic acid into a host cell genome include, for example, a retroviral vector, a lentiviral vector or an adeno-associated viral vector. Alternatively, an adenoviral vector is useful for introducing a nucleic acid that remains episomal into a host cell. Viral vectors are an efficient and versatile method of gene transfer in target cells and tissues. Additionally, high transduction efficiencies have been observed in many different cell types and target tissues.

For example, a retroviral vector generally comprises cis-acting long terminal repeats (LTRs) with packaging capacity for up to 6-10 kb of foreign sequence. The minimum cis-acting LTRs are sufficient for replication and packaging of a vector, which is then used to integrate the expression construct into the target cell to provide long term expression. Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:274-2378 (1989); Miller et al., J. Virol. 65:2220-2224 (1991); PCT/US94/05700; Miller and Rosman BioTechniques 7:980-990, 1989; Miller, A. D. Human Gene Therapy 1:5-14, 1990; Scarpa et al) Virology 180:849-852, 1991; Burns et al. Proc. Natl. Acad. Sci. USA 90:8033-8037, 1993.).

Various adeno-associated virus (AAV) vector systems have also been developed for nucleic acid delivery. AAV vectors can be readily constructed using techniques known in the art. See, e.g., U.S. Pat. Nos. 5,173,414 and 5,139,941; International Publication Nos. WO 92/01070 and WO 93/03769; Lebkowski et al. Molec. Cell. Biol. 8:3988-3996, 1988; Vincent et al. (1990) Vaccines 90 (Cold Spring Harbor Laboratory Press); Carter Current Opinion in Biotechnology 3:533-539, 1992; Muzyczka. Current Topics in Microbiol. and Immunol. 158:97-129, 1992; Kotin, Human Gene Therapy 5:793-801, 1994; Shelling and Smith Gene Therapy 1:165-169, 1994; and Zhou et al. J. Exp. Med. 179:1867-1875, 1994.

Additional viral vectors useful for delivering an expression construct of the invention include, for example, those derived from the pox family of viruses, such as vaccinia virus and avian poxvirus or an alphavirus or a conjugate virus vector (e.g. that described in Fisher-Hoch et al., Proc. Natl. Acad. Sci. USA 86:317-321, 1989).

The skilled artisan will be aware based on the foregoing description that the present invention also provides a composition comprising (i) a virus comprising a nucleic acid encoding a modulator of GDF-6 signaling in an IVD or a cell or tissue thereof sufficient to reduce, delay or prevent IVD degeneration in a subject and/or to induce and/or enhance IVD regeneration in a subject and a suitable carrier or excipient.

1.3 Cell-Based Modulators

The present invention also encompasses a composition comprising a cell, e.g., a stem cell comprising and/or expressing a modulator of GDF-6 signaling in an IVD or cell or tissue thereof. For example, the cell is transformed, transfected or transduced with a nucleic acid capable of expressing a peptide or polypeptide modulator of GDF-6 signaling, e.g., as described supra.

In one example, the cell is isolated from an IVD. For example, the cell is a nucleus pulposus cell or an annulus fibrosus cell. For example, the cell is isolated from a subject to be treated. For example, an IVD cell is isolated from a subject, e.g., using a syringe or by surgery. The cell is then transfected, transduced or transformed with a nucleic acid, e.g., an expression construct, capable of expressing a peptide or polypeptide modulator of GDF-6 signaling in said cell. Such a cell may then be introduced into a subject suffering from a spinal disorder and/or spinal pain.

In a preferred example, a cell is a stem cell. For example, a cell is a stem cell capable of differentiating into a cell in an IVD. Such a cell is useful for populating an IVD to which it is administered and reduce, prevent or delay IVD degeneration and/or enhance or induce IVD regeneration. Suitable stem cells will be apparent to the skilled artisan and include, a mesenchymal stem cell or a bone marrow stromal cell.

A suitable cell includes, for example, multipotent cells such as those described by Jiang, et al. (Nature, Vol. 418, p. 41-49, 2002).

Alternatively, a bone-marrow mesenchymal stem cell is isolated from a subject, e.g., a subject in need of treatment, and transformed, transfected or transduced with a nucleic acid capable of expressing a peptide or polypeptide modulator of GDF-6 signaling. Methods for isolating and/or administering a bone-marrow stromal cell will be apparent to the skilled artisan and/or described, for example, in Richardson et al., Stem Cells, 24: 707-716, 2006.

For example, a natural source of mesenchymal stem cells include bone marrow (e.g., with and without previous bleeding), peripheral blood (e.g., with and without enhancement from marrow), umbilical cord, fat, muscle, blood vessels, periosteum and perichondrium. Stem cells may be isolated from such a source by any suitable method, typically involving cell fractionation and concentration. Suitable methods are known in the art and include Ficoll-Paque methodology or concentration of mesenchymal stem cells using antibodies directed to mesenchymal stem cell markers which are immobilized, for example in an affinity chromatography column or to a substratum in a “panning” scheme.

Preferably a stem cell is allogenic (i.e., from the same species as a subject to be treated, and, preferably from the subject to be treated), as opposed to xenogenic (i.e., from a different species). If the cells are allogenic, but not autologous, it is preferred if the cells are of a similar tissue type (e.g. have similar MHC/HLA haplotypes). It is particularly preferred if the cells are autologous (i.e., are derived from the subject to which they are administered). Such autologous cells have the advantage of being less prone to rejection compared to other allogenic (or xenogenic) cells. Also, the use of autologous cells avoids any issue of “doping” (e.g., with “foreign” DNA). Accordingly, one example of the invention comprises obtaining a mesenchymal stem cell from a subject, transforming or transfecting the stem cell with a nucleic acid encoding a peptide or polypeptide modulator of GDF-6 signaling. It will be appreciated that some of the cells may be saved for use at a later date, and typically such cells are frozen under conditions that retains their viability. It will be appreciated that the cells may be obtained and enriched (expanded if necessary) before IVD degeneration in a subject, and kept for immediate administration when necessary.

Alternatively, or in addition, a bone-marrow stem cell comprising or expressing a modulator of GDF-6 signaling is cultured with a cell isolated from an IVD, e.g., a nucleus pulposus cell, prior to administration to a subject. Such co-cultivation induces differentiation of the stem cell into a cell similar to an IVD cell (Richardson et al., supra).

In one example, the cell is a chondrocyte, e.g., a progenitor cell capable of differentiating into an IVD cell, e.g., a nucleus pulposus cell or an annulus fibrosus cell. Chondrocytes generally express a marker such as, for example, Type II Collagen; Collagen IX; Aggrecan; Link Protein; S 100; or Biglycan. The skilled artisan will be aware of methods for producing or isolating such a chondrocyte. For example, as exemplified herein, contacting a mesenchymal stem cell, e.g., a bone marrow mesenchymal stem cell with GDF-6 for a time and under conditions sufficient for differentiation to occur causes the cell to differentiate into a chondrocyte. Such a chondrocyte is then suitable for administration to a subject to treat IVD degeneration and/or spinal pain and/or to induce IVD regeneration. Preferably, the chondrocyte has been modified to comprise or express a modulator of GDF-6 signaling.

In another example, an isolated stem cell, e.g., a bone marrow mesenchymal stem cell is contacted with a transforming growth factor (TGF)-β3 protein and/or a BMP-2 protein and/or a GDF-6 protein to induce differentiation into a chondrocyte cell, preferably a nucleus pulposus-like cell. Suitable methods for inducing differentiation are exemplified herein.

In one example, the composition described herein according to any embodiment, comprises a liquid suspension of cells comprising or expressing a modulator of GDF-6 signaling. For example, the liquid suspension is a suspension of cells in a medium that contains appropriate biological signals to encourage the differentiation of the mesenchymal stem cells into an IVD-type cell, and/or to discourage the differentiation of the cells into cell types that are not useful (e.g., bone tissue). The liquid suspension may be one which gels in situ, for example because of the temperature at the injury site of the patient, or because it is mixed with another agent that causes gelling.

In one example of the present invention, the cell additionally expresses a catalytic subunit of telomerase, e.g., encoded by a TERT gene or transcript. For example, the cell is genetically modified to express a catalytic subunit of telomerase. Such cells produce increased levels of collagen, e.g., collagen type 1 and/or collagen type 2. Suitable cells and methods for producing those cells are described, for example, in applicant's co-pending International Patent Application No. PCT/AU2006/000550.

In one example, a cell is isolated from a subject, e.g., an IVD cell and is transfected with an expression vector or expression construct comprising a nucleic acid encoding TERT operably linked to a promoter active in said cell. In one example, the cell is additionally transfected with a nucleic acid encoding a modulator of GDF-6 signaling. Methods for transfecting cells, e.g., IVD cells will be apparent to the skilled artisan and/or described herein and/or described in International Patent Application No. PCT/AU2006/000550. The resulting recombinant cell is then administered to a subject using a method described herein.

As will be apparent to the skilled artisan based on the foregoing description, the present invention also provides a method additionally comprising isolating or obtaining a stem cell. Such a method may additionally comprise producing a stem cell comprising or expressing a modulator of GDF-6 signaling, e.g., by performing a process comprising transforming or transfecting a stem cell with a nucleic acid that encodes a peptide or polypeptide modulator of GDF-6 signaling.

The present invention also provides a method for obtaining a chondrocyte or chondrocyte-like cell or a nucleus pulposus-like cell, said method comprising contacting a stem cell or a progenitor cell or a multipotent cell or a totipotent cell with an inducer of GDF-6 signaling, preferably, a GDF-6 polypeptide or active fragment thereof for a time and under conditions for the cell to differentiate, wherein following differentiation the cell is a chondrocyte or chondrocyte-like cell or a nucleus pulposus-like cell.

In one example, the stem cell or the progenitor cell or the multipotent cell is a mesenchymal stem cell, preferably a bone marrow mesenchymal stem cell.

In another example, the method additionally comprises contacting the stem cell or a progenitor cell or a multipotent cell or totipotent cell with a TGF-β3 polypeptide and/or a BMP2 polypeptide.

The present invention also provides a chondrocyte or chondrocyte-like cell or a nucleus pulposus-like cell produced by a method described herein according to any embodiment.

The present invention also provides a method of treating preventing or delaying or treating a spinal disorder and/or spinal pain in a subject, said method comprising administering a chondrocyte or chondrocyte-like cell or a nucleus pulposus-like cell produced by a method described herein according to any example to a subject suffering from a spinal disorder and/or spinal pain for a time and under conditions sufficient to reduce, delay or prevent intervertebral disc (IVD) degeneration in the subject and/or to induce and/or enhance intervertebral disc regeneration in the subject.

1.4 Assays to Identify a Modulator of GDF-6 Signaling

The skilled artisan will be aware of suitable methods for determining a compound capable of modulating GDF-6 signaling.

For example, a cell expressing a reporter gene, e.g., β-galactosidase or a fluorescent protein (e.g., green fluorescent protein) is placed under control of a BRE promoter, which is induced in the presence of GDF-6 signaling. The cell is then contacted with a test compound and the level of reporter gene expression is determined. A compound that enhances or reduces GDF-6 signaling compared to a cell that has not been contacted with a compound is considered a modulator of GDF-6 signaling. Such a method is described, for example, in Mazerbourgh et al., J. Biol. Chem., 280: 32122-32132, 2005.

Alternatively, or in addition, a cell is contacted with a test compound for a time and under conditions sufficient for GDF-6 signaling to occur and protein isolated from said cell. The level of phosphorylated Smad 1, Smad 5 and/or Smad 8 is then determined, e.g., by Western blotting using an anti-phospho Smad 1, Smad 5 or Smad 8 antibody (e.g., as available from Amersham Pharmacia). A compound that enhances or reduces the level of phosphorylated Smad 1, Smad 5 and/or Smad 8 in a cell compared to a cell that is not contacted with the compound is then considered a modulator of GDF-6 signaling. Such an assay is described, for example, in Mazerbourgh et al., supra.

In one example, the method described in either of the previous two paragraphs is performed in a cell from an IVD, e.g., a nucleus pulposus cell or an annulus fibrosus cell or in a cell in an IVD organ culture. Such an assay is useful for identifying a compound that modulated GDF-6 signaling in an IVD or cell or tissue thereof.

For example, GDF-6 signaling modulators may be identified by their ability to enhance or reduce the binding of two or more members of the GDF-6 signaling pathway to one another, e.g., a GDF-6 polypeptide to a GDF-6 receptor. For example, an assay is performed in which a labeled GDF-6 is contacted to a GDF-6 receptor in the presence or absence of a test compound. Following washing, the level of bound label is detected. A compound that enhances or reduces the level of label bound to the GDF-6 receptor is considered a modulator of GDF-6 signaling. Alternatively, or in addition, a GDF-6 signaling modulator is identified by their ability to enhance or inhibit protein interactions in the GDF-6 signaling cascade. For example, a reverse hybrid assay or forward hybrid assay is employed to identify a test compound inhibits or reduces or enhances an interaction between any of the following proteins: GDF-6 and/or MSX-1 and/or MSX-2 and/or BMPR-1A and/or BMPR-IB and/or BMPR-II and/or Smad-1 and/or Smad-5 and/or Smad-8 and/or Smad-4. Reverse hybrid methods will be apparent to the skilled artisan and/or described in Watt et al. (U.S. Ser. No. 09/227,652) or Erickson et al. (WO95/26400).

1.5 Assays to Determine Modulators of IVD Degeneration and/or Regeneration

The skilled artisan will also be aware of a suitable method to determine a compound and/or an amount of a compound that reduces, prevents or delays IVD degeneration and/or enhances IVD regeneration.

For example, an assay is performed in a cultured cell, e.g., a cell from an IVD, e.g., a nucleus pulposus cell or an annulus fibrosus cell or a similar cell or cell line, or a stem cell. For example, a cell is contacted with a test compound for a time and under conditions sufficient to modulate GDF-6 signaling and the level of a marker of IVD degeneration and/or regeneration, e.g., proteoglycan content and/or collagen content or production is determined. For example, a compound that enhances proteoglycan content of a cell and/or collagen content or production of a cell compare to a cell that is not contacted with the compound is considered reduces, prevents or delays IVD degeneration and/or enhances IVD regeneration.

Methods for determining the level of proteoglycan in a cell will be apparent to the skilled artisan and includes, for example, an assay to detect sulphated glycosaminoglycan using the metachromatic dye 1,9-dimethylmethylene blue (e.g., as described in Melrose et al., J Orthop Res 10:665-676, 1992; and Melrose et al., Matrix 14:61-75, 1994).

An assay for detecting collagen content of a cell includes, for example, an assay to detect hydroxyproline (e.g., essentially as described in Melrose et al., J Orthop Res 10:665-676, 1992; and Melrose et al., Matrix 14:61-75, 1994). Alternatively, or in addition, immunohistochemistry and/or immunofluorescence is used to detect the level of a collagen in a cell, e.g., Collagen Type I, Collagen Type II, Collagen Type IV, Collagen Type VI and Collagen Type X. Alternatively, or in addition, uptake of H-proline by a cell is indicative of the level of collagen synthesis by the cell.

Alternatively, or in addition a compound is administered to an animal model of IVD degeneration, such as for example, an animal model described herein. The effect of the compound is then determined, e.g., the water content of an IVD and/or the height of an IVD to which a compound has been administered is compared to the same parameter of an IVD to which the compound has not been administered. Improvement of the parameter indicates that the compound reduces, prevents or delays IVD degeneration and/or enhances IVD regeneration. Alternatively, the parameter in a treated IVD is compared to the same parameter in a non-degenerating IVD, and a similar level is indicative of a compound that reduces, prevents or delays IVD degeneration and/or enhances IVD regeneration.

Additional in vivo assays are exemplified herein.

2. Formulations

The GDF-6 signaling modulatory composition as described herein according to any example can be formulated readily for administration to a subject in need thereof e.g., by admixing the composition with a suitable carrier and/or excipient.

The terms “carrier” and “excipient” refer to carriers and excipients that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the formulation. In one example, the carrier does not produce significant local or systemic adverse effect in recipients at the dosages and concentrations employed for treatment.

Suitable carriers for this invention include those conventionally used, e.g., water, saline, aqueous dextrose, lactose, Ringer's solution, a buffered solution, hyaluronan and glycols are preferred liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

Preferred carriers and excipients do not adversely affect the ability of a GDF-6 signaling modulator to reduce, prevent or delay IVD degeneration and/or adversely affect the ability of a GDF-6 signaling modulator to enhance or induce IVD regeneration.

In one example, the carrier or excipient provides a buffering activity to maintain the compound at a suitable pH to thereby exert its biological activity, e.g., the carrier or excipient is phosphate buffered saline (PBS). PBS represents an attractive carrier or excipient because it interacts with compounds minimally and permits rapid release of the compound. In such a case, the composition of the invention may be produced as a liquid or direct application to an IVD or a region surrounding or adjacent to an IVD, e.g., by injection.

In another example, the composition of the invention is formulated with a co-polymer. For example, Puolakkainen et al., J. Surg. Res., 58: 321-329 describe a poly(ethylene oxide)-poly(propylene oxide) block copolymer designated Pluronic F-127. Pluronic F-127 has been used as a carrier for a variety of peptides and proteins in addition to nucleic acid based compounds. This carrier exhibits thermoreversability, relative inertness toward protein and nucleic acid and low toxicity.

In a further example, the carrier is a hydrogel. In this respect, a hydrogel is a three dimensional network of cross-linked hydrophilic polymers in the form of a gel substantially composed of water, preferably but not limited to gels being greater than 90% water. Hydrogel can carry a net positive or net negative charge, or may be neutral. A typical net negative charged hydrogel is alginate. Hydrogels carrying a net positive charge may be typified by extracellular matrix components such as collagen and laminin. Examples of commercially available extracellular matrix components include Matrigel™ and Vitrogen™. An example of a net neutral hydrogel is highly crosslinked polyethylene oxide, or polyvinyalcohol. For example, biopol hydrogel is a poly(ethylene oxide) cross-linked hydrogel that interacts with aqueous solutions and swells to an equilibrium value, retaining a significant portion of the aqueous solution within its structure. Hydrogels have been shown to be suitable for delivery of a number of compounds, including proteins or peptides (Pitt et al., Int. J. Pharm., 59: 173, 1990.

In a further example, the carrier is a hydroxypropyl methylcellulose (HPMC) or a hydroxypropyl cellulose (HPC). Such carriers may be formulated as a liquid, a gel or a cream. Optionally, the carrier additionally comprises n-methyl-2-pyrrolidine (NMP) to enhance uptake of a topical composition therein.

In the case of a cell-based therapeutic a preferred carrier includes a hyaluronan gel. Alternatively, or in addition, a suitable hydrogel for administration of a cell or peptide or nucleic acid is described in US Patent Publication No. 20060115457.

In a further example, a GDF-6 signaling modulator is formulated with polyethylene glycol (PEG) as a delivery material. The PEG group(s) may be of any convenient molecular weight and may be linear or branched. For example, the composition comprises PEG. Alternatively, or in addition, the GDF-6 signaling modulator is covalently linked to the PEG group(s). Methods for PEGylating proteins are known in the art.

In another example, the clearance of a GDF-6 signaling modulator is delayed to extend the effective half-life of the GDF-6 modulator at the site of action (i.e., within an IVD, e.g., within a nucleus pulposus and/or within a region of an IVD defined by an annulus fibrosus) by appropriate formulation e.g., for sustained-release of the GDF-6 signaling modulator and/or for slow delivery of the GDF-6 signaling modulator. Formulations comprising gels, hydrogels, microspheres or biocompatible polymers, including bioresorbable polymers, are particularly suited to such applications. Suitable formulations for such applications may comprise, for example, polylactic/polyglycolic acid polymers, liposomes, collagen, polyethylene glycol (PEG), hyaluronic acid/fibrin matrices, hyaluronic acid, fibrin, chitosan, gelatin, SABER™ System (sucrose acetate isobutyrate (SAIB)), DURIN™ (biodegradabale polymer for drug loaded implants), MICRODUR™ (biodegradable polymers/microencapsulation) and DUROS™ (mini-osmotic pump). Biocompatible polymeric materials include elastic or elastomeric materials, hydrogels or other hydrophilic polymers, or composites thereof. Suitable elastomers include silicone, polyurethane, copolymers of silicone and polyurethane, polyolefins, such as polyisobutylene and polyisoprene, neoprene, nitrile, vulcanized rubber and combinations thereof. Suitable hydrogels include natural hydrogels, and those formed from polyvinyl alcohol, acrylamides such as polyacrylic acid and poly(acrylonitrile-acrylic acid), polyurethanes, polyethylene glycol, poly(N-vinyl-2-pyrrolidone), acrylates such as poly(2-hydroxy ethyl methacrylate) and copolymers of acrylates with N-vinyl pyrrolidone, N-vinyl lactams, acrylamide, polyurethanes and polyacrylonitrile, or may be other similar materials that form a hydrogel. The hydrogel materials may further be cross-linked to provide further strength to the implant. Examples of polyurethanes include thermoplastic polyurethanes, aliphatic polyurethanes, segmented polyurethanes, hydrophilic polyurethanes, polyether-urethane, polycarbonate-urethane and silicone polyetherurethane. Other suitable hydrophilic polymers include naturally occurring materials such as glucomannan gel, hyaluronic acid, polysaccharides, such as cross-linked carboxyl-containing polysaccharides, and combinations thereof.

Formulations of the present invention can be subjected to conventional pharmaceutical expedients, such as sterilization, and can contain a conventional pharmaceutical additive, such as a preservative and/or a stabilizing agent and/or a wetting agent and/or an emulsifying agent and/or a salt for adjusting osmotic pressure and/or a buffer and/or other additives known in the art. Other acceptable components in the composition of the invention include, but are not limited to, isotonicity-modifying agents such as water and/or saline and/or a buffer including phosphate, citrate, succinate, acetic acid, or other organic acids or their salts.

In one example, a formulation of the invention includes one or more stabilizers, reducing agents, anti-oxidants and/or anti-oxidant chelating agents. The use of buffers, stabilizers, reducing agents, anti-oxidants and chelating agents in the preparation of compositions, is known in the art and described, for example, in Wang et al. J. Parent. Drug Assn. 34:452-462, 1980; Wang et al. J. Parent. Sci. Tech. 42:S4-S26(Supplement), 1988. Suitable buffers include acetate, adipate, benzoate, citrate, lactate, maleate, phosphate, tartarate, borate, tri(hydroxymethyl aminomethane), succinate, glycine, histidine, the salts of various amino acids, or the like, or combinations thereof. Suitable salts and isotonicifiers include sodium chloride, dextrose, mannitol, sucrose, trehalose, or the like. Where the carrier is a liquid, it is preferred that the carrier is hypotonic or isotonic with oral, conjunctival, or dermal fluids and has a pH within the range of 4.5-8.5. Where the carrier is in powdered form, it is preferred that the carrier is also within an acceptable non-toxic pH range.

In another example, a formulation as described herein according to any example additionally comprises a liposome carrier or excipient to facilitate uptake of a GDF-6 signaling modulator into a cell. Liposomes are considered to interact with a cell by stable absorption, endocytosis, lipid transfer, and/or fusion (Egerdie et al., J. Urol. 142:390, 1989). For example, liposomes comprise molecular films, which fuse with cells and provide optimal conditions for wound healing (K. Reimer et al., Dermatology 195(suppl. 2):93, 1999). Generally, liposomes have low antigenicity and can be used to encapsulate and deliver components that cause undesirable immune responses in patients (Natsume et al., Jpn. J. Cancer Res. 91:363-367, 2000)

For example, anionic or neutral liposomes often possess excellent colloidal stability, since substantially no aggregation occurs between the carrier and the environment. Consequently their biodistribution is excellent, and their potential for irritation and cytotoxicity is low.

Alternatively, cationic liposomal systems, e.g. as described in Mauer et al., Molecular Membrane Biology, 16:, 129-140, 1999 or Maeidan et al., BBA 1464: 251-261, 2000 are useful for delivering compounds into a cell. Such cationic systems provide high loading efficiencies. Moreover, PEGylated cationic liposomes show enhanced circulation times in vivo (Semple BBA 1510, 152-166, 2001).

Amphoteric liposomes are a recently described class of liposomes having an anionic or neutral charge at pH 7.4 and a cationic charge at pH 4. Examples of these liposomes are described, for example, in WO 02/066490, WO 02/066012 and WO 03/070735. Amphoteric liposomes have been found to have a good biodistribution and to be well tolerated in animals and they can encapsulate nucleic acid molecules with high efficiency.

U.S. Ser. No.09/738,046 and U.S. Ser. No. 10/218,797 describe liposome formulations suitable for the delivery of peptides or proteins into a cell.

In one example, a carrier or excipient comprises poly(methyl methacrylate) (PMMA), optionally chondroitin sulphate (CS), an amphiphilic macromonomer (MT), 2-hydroxyethyl methacrylate (HEMA) and, optionally, acrylic acid (AA), as described in Larraz et al., J. Tissue Eng. and Regen. Med., 1: 120-127, 2007.

In the case of a nucleic acid based modulator of GDF-6 signaling a carrier or excipient preferably comprises a lipid-based agent, e.g., a cationic lipid. For example, the carrier or excipient comprises a cationic lipid, such as 2,3-dioleyloxy-N-[2(sperminecarboxyamido)ethyl]-N,N-dimethyl-1-propanaminium trifluoroacetate), Lipofectin, Lipofectace, DOTAP, DOTMA (N-[1-(2,3-dioleyloxy)propyl]-N,N,N-trimethylammonium chloride), CDAB (cetyldimethylethylammonium bromide), CTAB (cetyltrimethylethylammonium bromide), DDAB (dimethyldioctadecylammonium bromide), MBC (methylbenzethonium chloride), FuGENE (Roche) or stearylamine. Other suitable lipids are disclosed, for example in U.S. Pat. No. 5,855,910, International Patent Publication No. WO 02/072068 and International Patent Publication No. WO 00/30444.

3. Medical Devices

The present invention also provides a medical device comprising an amount of a modulator of GDF-6 signaling in an IVD or a cell or tissue thereof sufficient to reduce, delay or prevent IVD degeneration in a subject and/or to induce and/or enhance IVD regeneration in a subject or comprising a composition as described herein according to any embodiment.

For example, the medical device is a syringe comprising a composition described herein according to any embodiment.

In one example, the medical device comprising the modulator of GDF-6 signaling is a device comprising a delivery conduit having a proximal end attachable to a source of the GDF-6 signaling modulator and an emitter structure at a distal end of the delivery conduit, wherein the emitter structure defines a plurality of spaced discharge apertures through which the GDF-6 signaling modulator is delivered to a plurality of sites or in a patterned manner within the IVD and/or within a nucleus pulposus and/or adjacent to a nucleus pulposus and/or within a region of an IVD defined by an annulus fibrosus and wherein the emitter structure is configured to promote diffuse distribution of the GDF-6 signaling modulator within or throughout the IVD, e.g., within a nucleus pulposus and/or adjacent to a nucleus pulposus and/or within a region of an IVD defined by an annulus fibrosus. Preferably, the apertures are dimensioned to achieve a substantially uniform discharge rate of the GDF-6 signaling modulator or composition of the invention through all of the apertures.

The emitter structure of such a device may be steerable. Thus the emitter structure may include a pull wire attached to the emitter structure, either within a lumen of the emitter structure or embedded in a wall of the emitter structure. Instead, the device may include a guide element for guiding the emitter structure into an operative position at the site in which diffuse distribution of the GDF-6 signaling modulator or composition of the invention is promoted. The guide element may be a guide wire of a preformed shape extending through a lumen of the emitter structure. The guide wire may, for example, be of a shape memory alloy such as Nitinol®.

A wall of the emitter structure in such a device may be reinforced to maintain the integrity of the emitter structure in situ. More particularly, the emitter structure may be reinforced to inhibit collapsing of the lumen of the emitter structure as a result of pressure applied to the IVD or to the nucleus pulposus of an IVD or pressure applied adjacent to or surrounding the IVD.

For example, the emitter structure of such a device may be shaped to form a loop at the site of the IVD or the nucleus pulposus or a region of the IVD defined by an annulus fibrosus in the patient's body. In another embodiment, the emitter structure may be substantially annular. In still a further embodiment, the emitter structure may be forked into a plurality of branches. By “forked” is meant more than two branches.

The emitter structure of such a device may also be formed integrally with the delivery conduit as a one-piece unit. The device may include a working cannula via which the unit is able to be delivered into an IVD and/or into a nucleus puplosus and/or into a region of an IVD defined by an annulus fibrosus percutaneously in a minimally invasive manner.

The emitter structure of such a device may also carry at least one radio-opaque marker.

Each aperture of such a device may include a flow control device for inhibiting back flow of the GDF-6 signaling modulator or composition of the invention into the emitter structure. The flow control device may be a one-way valve. The flow control device may be adjusted to improve the flow of formulations of higher viscosity.

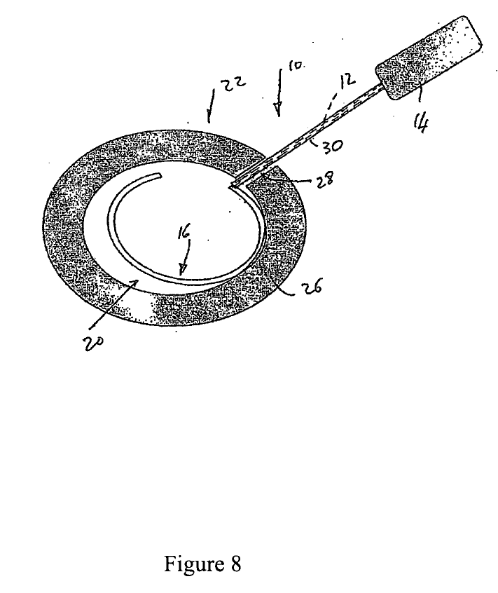

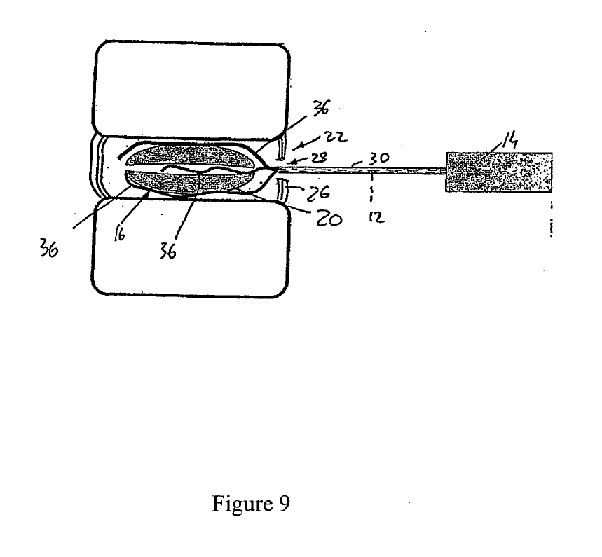

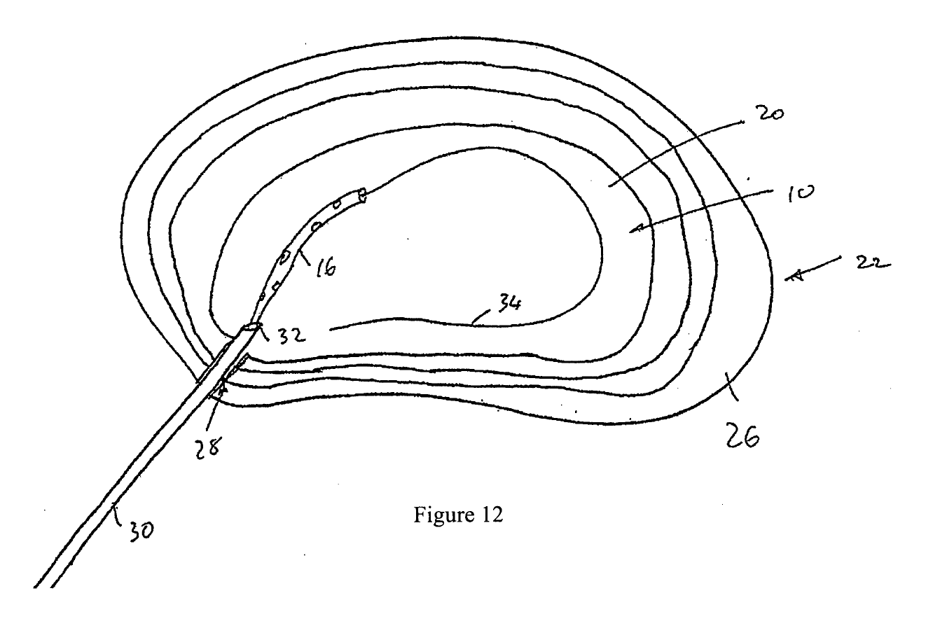

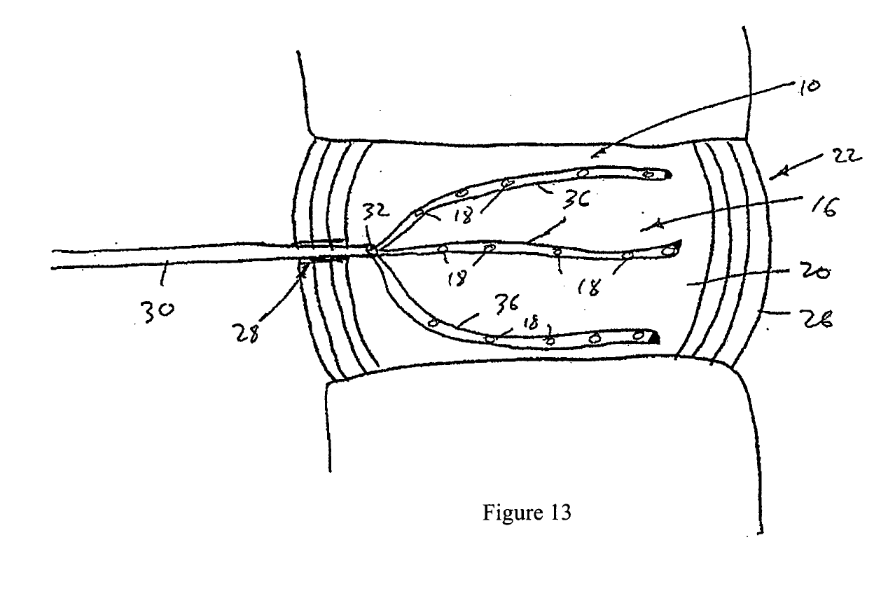

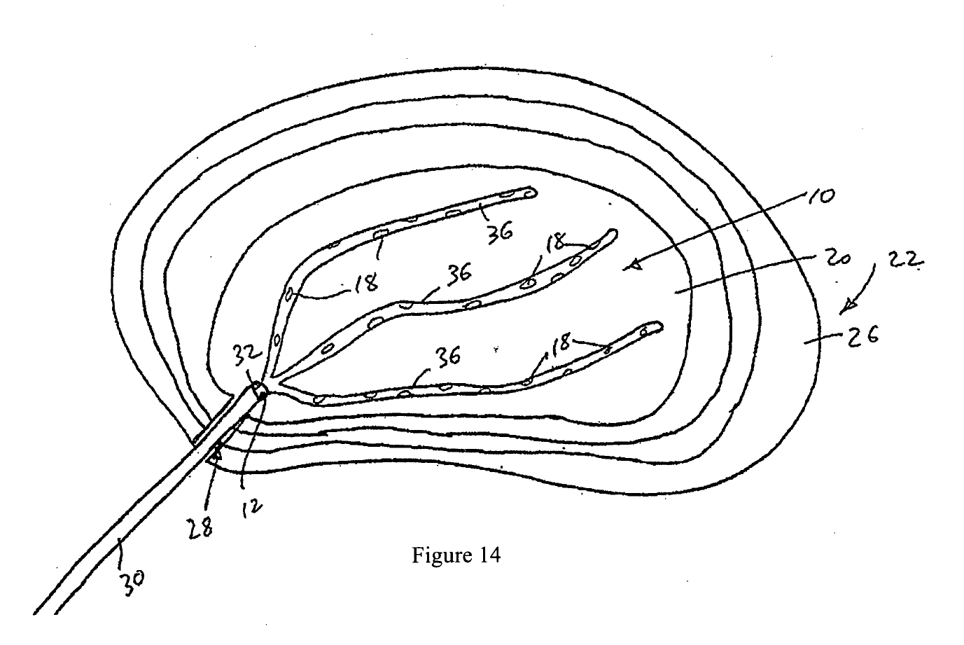

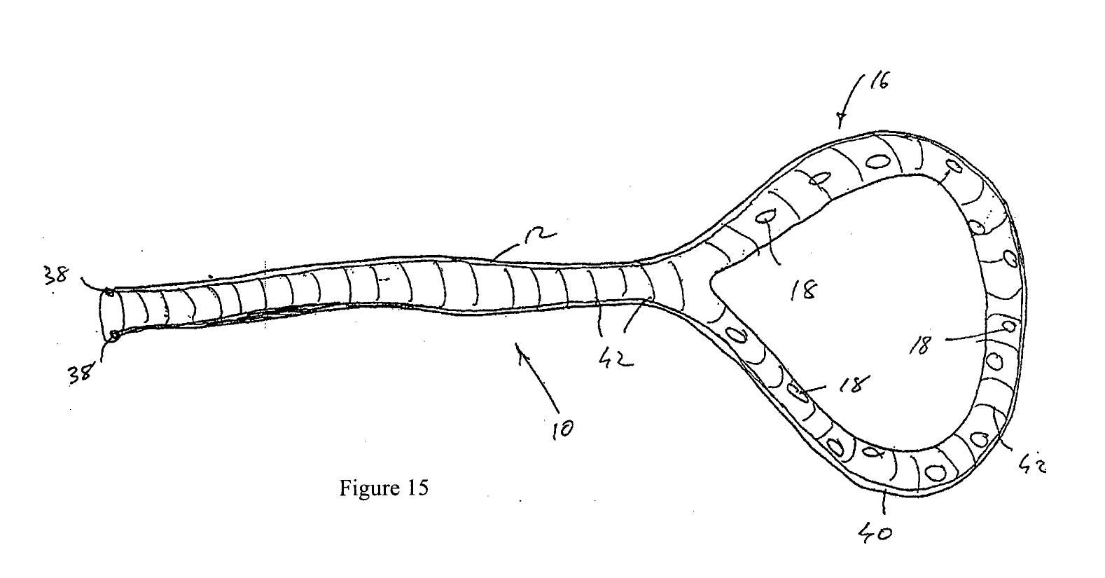

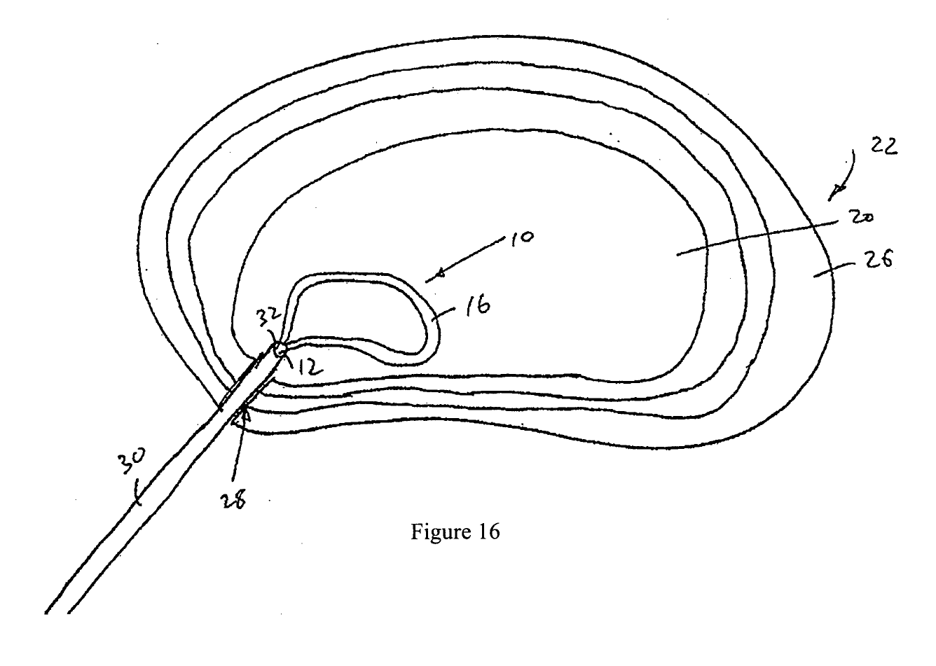

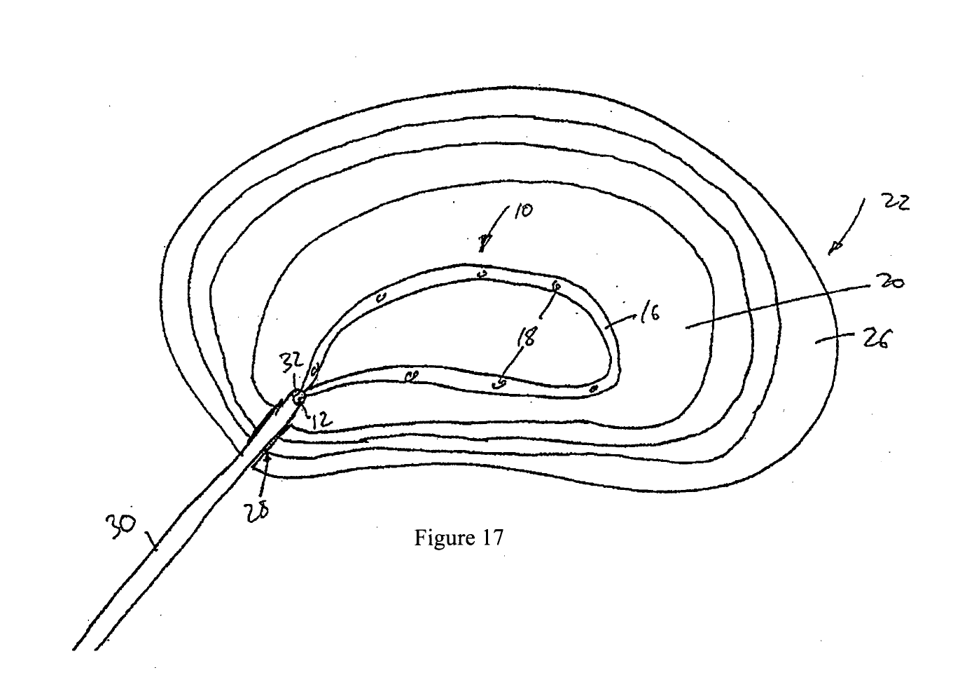

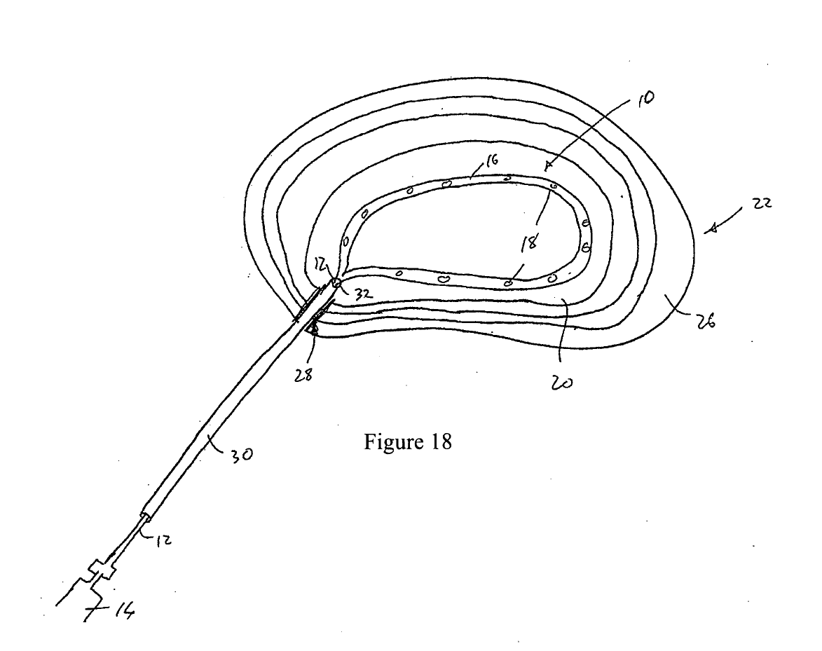

The present invention clearly encompasses a system for the delivery of a GDF-6 signaling modulator or a composition of the present invention into an IVD and/or into a nucleus puplosus and/or into a region of an IVD defined by an annulus fibrosus and/or adjacent to at least a portion of a nucleus pulposus, said system comprising a device as described according to any example hereof, for example with reference to any one or more of FIGS. 8 to 18, and a source of the GDF-6 signaling modulator or composition of the present invention attached to the proximal end of the delivery conduit of the device. The source of the GDF-6 signaling modulator or composition can be a fluid dispenser, such as a syringe.

In a further example, the present invention provides a delivery system for administering the composition or modulator comprising:
(i) a dispenser defining a reservoir and an outlet port in communication with the reservoir;
(ii) a high density, immiscible, non-reactive, biocompatible displacement fluid comprising the modulator or composition, said fluid being contained within the reservoir ; and
(iii) a displacement device arranged in the reservoir for displacing the fluid through the outlet port of the dispenser.

In this example, a receptacle may be provided for the fluid, the receptacle having a mounting formation for mounting the receptacle to the dispenser so that an interior of the receptacle is in communication with the outlet port of the dispenser. The receptacle may comprise a cannula with at least one discharge opening. Preferably, the cannula is elongate having a side wall defining a plurality of axially spaced discharge openings. For example, each discharge opening may include an occluding device for inhibiting back flow of the fluid into the interior of the cannula. Preferably, at least some of the openings open out into a recessed region of the side wall of the cannula. The cannula may be shaped and dimensioned to access a plurality of sites simultaneously. In accordance with these examples, the cannula is preferably flexible to facilitate direction to a desired location in a patient's body.

In this example, it is also preferred that the delivery system comprises a reaming tool for forming a passage through bone at a site in the patient's body into which the receptacle is to be inserted. The reaming tool may be steerable.

In a further example, a delivery system comprises:
(i) an elongate body defining a lumen;
(ii) at least one opening defined in the body through which the modulator or composition can be discharged; and
(iii) an occluding device contained in a receptacle in register with at least one of said openings, said occluding device being for closing off the opening(s) to thereby inhibit back flow of the modulator or composition into the lumen of the body after being discharged through the opening(s).

The body may have a mounting formation for mounting to a dispenser so that an interior of the body is in communication with an outlet port of the dispenser. The body may comprise a cannula having a side wall defining a plurality of axially spaced discharge openings. A proportion of said plurality of openings may open out into a recessed region of the side wall of the cannula. The cannula may be shaped and dimensioned to access a plurality of sites simultaneously. The cannula may be flexible to be able to be directed to a desired location in a patient's body.

In a further example, a cannula is provided that comprises the modulator or composition, wherein the cannula is adapted for insertion into a site in the vertebral column of the subject, and mounted on a dispensing device, and a high density, immiscible, non-reactive, biocompatible displacement fluid is provided within a reservoir of the dispensing device to discharge the modulator or composition from the cannula.

In a further example, the present invention provides a medical device for the delivery of a GDF-6 signaling modulator or composition of the present invention into an IVD and/or into a nucleus puplosus and/or into a region of an IVD defined by an annulus fibrosus,and/or adjacent to at least a portion of a nucleus pulposus wherein the medical device comprises a delivery conduit having a proximal end attachable to a source of the GDF-6 signaling modulator or composition of the present invention and an emitter structure at a distal end of the delivery conduit, wherein the emitter structure is at least partially receivable within an interior of the IVD, preferably within a nucleus pulposus and/or within a region of an IVD defined by an annulus fibrosus and/or adjacent to at least a portion of a nucleus pulposus and defining a plurality of spaced discharge apertures through which the GDF-6 signaling modulator or composition is delivered into the IVD and/or into the nucleus puplosus and/or into the region of an IVD defined by an annulus fibrosus and/or adjacent to at least a portion of a nucleus pulposus and wherein the emitter structure is configured to extend about a part of the IVD, e.g., a nucleus pulposus or a region of an IVD defined by an annulus fibrosus and/or adjacent to at least a portion of a nucleus pulposus to thereby promote administration of the GDF-6 modulator or composition to a plurality of sites or in a patterned manner within the IVD and/or nucleus puplosus and/or region of an IVD defined by an annulus fibrosus and/or adjacent to at least a portion of a nucleus pulposus, e.g., to promote diffuse or substantially uniform distribution of the GDF-6 signaling modulator or composition throughout the IVD and/or nucleus puplosus and/or region of an IVD defined by an annulus fibrosus.

In another example, the medical device comprises a synthetic or natural-sourced matrix configured in size and shape to fit the defect site to be repaired, e.g., an IVD or a nucleus pulposus or a region of an IVD defined by an annulus fibrosus.

In another example, the medical device comprises a spinal implant. For example, the spinal implant is for treating an IVD while retaining an intact annulus fibrosus, the device including a compressible fibrous body configurable to a compressed state for passage through an opening in the annulus fibrosus and into a disc cavity defined by the annulus fibrosus. The body is also configurable to an expanded state to reside within the disc cavity and have a dimension greater than the opening so as to resist expulsion from the opening. The body incorporates an effective amount of a modulator of GDF-6 signaling, or a cell (e.g., a stem cell, a nucleus pulposus cell or an annulus fibrosus cell comprising or expressing said modulator of GDF-6 signaling) or a composition of the present invention. Such a device is described, for example, in US Patent Publication No. 20020173851.

Alternatively, the device comprises a fibrous body sized for passage through an opening in the annulus fibrosus and into a disc cavity defined by the annulus fibrosus. The body is formed of fibers having coated thereon a solid carrier matrix incorporating a modulator of GDF-6 signaling or a composition of the present invention.

A medical device encompassed by the present invention, especially any implant, may be partially or completely bioresorbable. In addition, the body may be sized and configured to provide temporary or permanent prosthetic function, by being dimensioned to participate in the distribution of compressive loads between adjacent vertebral bodies. For example, the body may be adapted to physically maintain a space in the disc as new tissue is generated, and provide a substrate for tissue ingrowth which locks the implant in place and reinforces regenerated tissues to help maintain disc space height. Alternatively, the body may be non-prosthetic, while delivering a modulator of GDF-6 signaling. In such non-prosthetic applications, the device can be dimensioned, or can be formed of a material having compressive properties, such that it does not participate in the distribution of loads between the adjacent vertebral bodies.

A spinal disc implant contemplated by the present invention is fabricated in any of a variety of shapes, as desired for a particular application. Whilst, the implant may assume a variety of shapes, it is typically shaped to conform to the shape of the natural nucleus pulposus, at least when in its hydrated and/or relaxed configuration. Thus, the implant is preferably substantially elliptical when in its hydrated and/or relaxed configuration. In other forms of the invention, the shape of the implant in its hydrated and/or relaxed configuration is generally annular-shaped, cylindrical-shaped, or otherwise shaped as required to conform to a cavity in an IVD.

Suitable spinal disc implants are also shaped in a manner to allow easy implantation into a spinal disc nucleus space. Accordingly, the implant may have a narrow, tubular shape when in its dehydrated and/or straightened configuration, and may include at least one narrow or pointed end to facilitate implantation through a small annulus hole.

A spinal disc implant for use in the invention may be formed from a wide variety of biocompatible polymeric materials, including elastic materials, such as elastomeric materials, hydrogels or other hydrophilic polymers, or composites thereof. Suitable elastomers include silicone, polyurethane, copolymers of silicone and polyurethane, polyolefins, such as polyisobutylene and polyisoprene, neoprene, nitrile, vulcanized rubber and combinations thereof. Suitable hydrogels include natural hydrogels, and those formed from polyvinyl alcohol, acrylamides such as polyacrylic acid and poly(acrylonitrile-acrylic acid), polyurethanes, polyethylene glycol, poly (N-vinyl-2-pyrrolidone), acrylates such as poly(2-hydroxy ethyl methacrylate) and copolymers of acrylates with. N-vinyl pyrrolidone, N-vinyl lactams, acrylamide, polyurethanes and polyacrylonitrile, or may be other similar materials that form a hydrogel. The hydrogel materials may further be cross-linked to provide further strength to the implant. Examples of polyurethanes include thermoplastic polyurethanes, aliphatic polyurethanes, segmented polyurethanes, hydrophilic polyurethanes, polyether-urethane, polycarbonate-urethane and silicone polyetherurethane. Other suitable hydrophilic polymers include naturally occurring materials such as glucomannan gel, hyaluronic acid, polysaccharides, such as cross-linked carboxyl-containing polysaccharides, and combinations thereof. The nature of the materials employed to form the elastic body should be selected so the formed implants have sufficient load bearing capacity. In preferred embodiments, a compressive strength of at least about 0.1 Mpa is desired, however compressive strengths in the range of about 1 Mpa to about 20 Mpa are more preferred.

Additional suitable implants will be apparent to the skilled artisan and are described, for example, in International Application No. PCT/AU2006/000267.

4. Modes of Administration

The present invention contemplates any mode of administration of a modulator of GDF-6 signaling or a composition as described herein according to any example in a method of treatment. For example, the present invention contemplates administration surgically or by injection or a combination thereof. Those skilled in the art will recognize that, notwithstanding implants and stents may be delivered readily by surgical means, and injectable formulations are generally delivered to the IVD region by injection, these modes of administration are not mutually exclusive. For example, an implant or stent may be amenable by virtue of its, small size, flexibility or other physicochemical properties to be administered by injection.

Preferred means for injection of a GDF-6 modulatory composition include intravenous, subcutaneous, percutaneous, intramuscular and intradiscal routes. (e.g., intradiscal injection or intradiscal implant), the only requirement being that the GDF-6modulatory compound is delivered to the region of the IVD in an amount effective to modulate the GDF-6 signaling pathway therein. Preferably, the composition or GDF-6 modulator is delivered into an IVD, more preferably into a nucleus puplosus and/or a region of an IVD defined by an annulus fibrosus.

For example, a polypeptide or protein modulator or cell expressing same is injected into an IVD, preferably, into a nucleus pulposus and/or adjacent to at least a portion of a nucleus pulposus and/or into a region of an IVD defined by an annulus fibrosus or into a region surrounding or adjacent to an IVD. Preferably, a polypeptide or protein modulator or cell expressing same or composition as described herein according to any example is administered to a plurality of sites or locations or positions within an IVD, preferably within a nucleus pulposus and/or within a region of an IVD defined by an annulus fibrosus. Preferably, following a period of time sufficient to diffusion of the composition or modulator, the modulator or composition is distributed substantially uniformly or uniformly within an IVD and/or within a nucleus pulposus and/or within a region of an IVD defined by an annulus fibrosus. For example, a suitable route of administration is intradiscal administration, intrathecal administration or intraganglionic administration (see, e.g., TEXTBOOK OF PAIN, Wall and Melzack, Eds. Harcourt Brace, 4th Ed, 1999). One particularly useful method involves administering by discography as generally described by Carragee et al., Spine 24): 2542-2547, 1999.

In another example, a modulator of GDF-6 signaling or a composition as described herein according to any example is administered by intradiscal injection or intradiscal implant.

In another example, a modulator of GDF-6 signaling is administered to or within an IVD and/or to or within a nucleus pulposus and/or adjacent to at least a portion of a nucleus pulposus and/or to or within a region of an IVD defined by an annulus fibrosus using a medical device as according to any example hereof that comprises the GDF-6 signaling modulator or composition of the present invention such as, for example, in accordance with Example 9. For example, the GDF-6 signaling modulator can be administered to or within an IVD or to or within a nucleus pulposus and/or adjacent to at least a portion of a nucleus pulposus and/or to or within a region of an IVD defined by an annulus fibrosus by a process comprising:

accessing the region of the IVD such as by surgical intervention or by injection e.g., percutaneously using a cannula;

providing a medical device comprising a modulator of GDF-6 signaling or a composition of the present invention wherein the medical device comprises a delivery conduit having a proximal end attachable to a source of the GDF-6 signaling modulator or the composition and an emitter structure at a distal end of the delivery conduit, wherein the emitter structure defines a plurality of spaced discharge apertures through which the GDF-6 signaling modulator or composition is deliverable to the IVD or to the nucleus pulposus and/or adjacent to at least a portion of a nucleus pulposus and/or to the region of an IVD defined by an annulus fibrosus and wherein the emitter structure is configured to administer the GDF-6 signaling modulator or composition to a plurality of sites within the IVD and/or nucleus puplosus and/or region of the IVD defined by the annulus fibrosus;

inserting the emitter structure of the medical device at least partially into the accessed region of the IVD;

manipulating the emitter structure so that the emitter structure at least partially surrounds or is positioned within the nucleus pulposus and/or region of the IVD defined by the annulus fibrosus and/or adjacent to at least a portion of a nucleus pulposus; and

discharging the GDF-6 signaling modulator or composition through the apertures so as to administer the GDF-6 signaling modulator or composition to a plurality of sites within the IVD and/or nucleus puplosus and/or region of the IVD defined by the annulus fibrosus and/or adjacent to at least a portion of a nucleus pulposus, e.g., to promote diffuse and preferably uniform distribution of the GDF-6 signaling modulator or composition within the IVD and/or nucleus puplosus and/or region of the IVD defined by the annulus fibrosus and/or adjacent to at least a portion of a nucleus pulposus.

For example, the GDF-6 signaling modulator or composition of the present invention can be administered to or within an IVD or to or within a nucleus pulposus and/or to or within a region of an IVD defined by an annulus fibrosus and/or adjacent to at least a portion of a nucleus pulposus by a process comprising:
accessing the region of the IVD such as by surgical intervention or by injection e.g., percutaneously using a cannula;
providing a medical device comprising a modulator of GDF-6 signaling or composition of the present invention wherein the medical device comprises a delivery conduit having a proximal end attachable to a source of the GDF-6 signaling modulator or composition and an emitter structure at a distal end of the delivery conduit, wherein the emitter structure is at least partially receivable within an interior of the IVD and defining a plurality of spaced discharge apertures through which the GDF-6 signaling modulator or composition is delivered to a part of the IVD, preferably to a nucleus pulposus and/or a region of an IVD defined by an annulus fibrosus and/or adjacent to at least a portion of a nucleus pulposus and wherein the emitter structure is configured to administer the GDF-6 signaling modulator or composition to a plurality of sites within the IVD and/or nucleus puplosus and/or region of the IVD defined by the annulus fibrosus and/or adjacent to at least a portion of a nucleus pulposus;
inserting the emitter structure of the medical device at least partially into the accessed region of the IVD;
manipulating the emitter structure so that the emitter structure at least partially surrounds or is positioned within the nucleus pulposus and/or region of the IVD defined by the annulus fibrosus and/or adjacent to at least a portion of a nucleus pulposus; and
discharging the GDF-6 signaling modulator through the apertures so as to administer the GDF-6 signaling modulator or composition to a plurality of sites within the IVD and/or nucleus puplosus and/or region of the IVD defined by the annulus fibrosus and/or adjacent to at least a portion of a nucleus pulposus, e.g., to promote diffuse and preferably uniform distribution of the GDF-6 signaling modulator or composition within the IVD and/or nucleus puplosus and/or region of the IVD defined by the annulus fibrosus and/or adjacent to at least a portion of a nucleus pulposus..

In use, it is preferred to guide an emitter structure supra to the site of the IVD in an inoperative configuration and, when positioned at the site, to configure the emitter structure in an operative configuration to thereby at least partially surround or be positioned within a nucleus pulposus and/or region of the IVD defined by the annulus fibrosus. Thus, the emitter structure can be guided into its operative configuration.

It is also preferred to apply a substantially uniform flow rate of the GDF-6 modulator or composition through all of the apertures of the emitter structure.

The present invention further encompasses the performing of an annulotomy in an annulus of the IVD and distributing the GDF-6 modulator or composition to a plurality of sites or in a patterned manner within the disc, and/or the implanting a medical device comprising the GDF-6 modulator or composition. Implantations may be performed following a nucleotomy or without the need for a nucleotomy depending on the state of degeneration of the disc.

Preferred means for deploying the emitter structure include endoscopic visualization means and/or by fluoroscopic guidance techniques. As will be known to the skilled artisan, such techniques may require formulations of the GDF-6 signaling modulator that include at least one radio-opaque marker.

It is also preferred to substantially prevent back-flow of the GDF-6 signaling modulator or composition through the apertures in the emitter structure when delivery of the composition has been completed.

In the case of a nucleic acid modulator of GDF-6 signaling, the modulator or composition may be administered by particle bombardment or by liposome mediated delivery. Alternative methods for the delivery of nucleic acid modulators include, for example, microseeding (Erikkson et al., J. Surg. Res., 78: 85-91, 1998), microfabricated needles (Henry et al., J. Pharm. Sci., 87: 922-925, 1998), puncture mediated DNA transfer (Ciernik et al., Hum. Gene Ther., 7: 893-899, 1996), lipid or liposome mediated delivery (Li et al., In Vitro Cell Devel., Biol., 29A: 258-260, 1993; or Alexander et al., Hum. Mol. Genet., 4: 2279-2285, 1995).

Alternatively, or in addition, a nucleic acid modulator is delivered by a viral-mediated process, e.g., an adenovirus or a retrovirus.

It is to be understood that notwithstanding the surgical procedures described herein for administering a modulator or composition are performed so as to be minimally invasive procedures, it may necessary to produce one or more openings or fissures in the annulus fibrosus. Several means are known to those skilled in the art for closing or otherwise repairing such openings, including e.g., suturing the annulus fibrosis as described for example in WO/2006/023348, or inserting a porous or deformable implant such as a plug into the annulus which may then becomes anchored rigidly in the end-plates as described for example in WO/2005/020859 or WO 2008/045638 or U.S. Pat. No. 6,224,630.

5. Dosage of Therapeutic GDF-6 Modulatory Composition

Selecting an administration regimen for a therapeutic composition depends on several factors, including the serum or tissue turnover rate of the entity, the level of symptoms, the immunogenicity of a modulator of GDF-6 signaling, and the accessibility of the target cells in the biological matrix. Preferably, an administration regimen maximizes the amount of therapeutic compound delivered to the patient consistent with an acceptable level of side effects. Accordingly, the amount of composition delivered depends in part on the particular entity and the severity of the condition being treated. Guidance in selecting appropriate doses of peptides are available (see, e.g., Milgrom, et al. New Engl. J. Med. 341:1966-1973, 1999; Slamon, et al. New Engl. J. Med. 344:783-792, 2001; Beniaminovitz, et al. New Engl. J. Med. 342:613-619, 2000; Ghosh, et al. New Engl. J. Med. 348:24-32, 2003; or Lipsky, et al. New Engl. J. Med. 343:1594-1602, 2000).

In one example, a modulator of GDF-6 signaling is administered in a single bolus dosage. Alternatively, a peptide or polypeptide is provided, for example, by continuous infusion, or by doses at intervals of, e.g., one day, one week, or 1-7 times per week. Preferably, a modulator of GDF-6 signaling or a composition comprising said modulator is administered to a plurality of sites or in a patterned manner within an IVD, preferably within a nucleus pulposus or within a region of an IVD defined by an annulus fibrosus and/or adjacent to at least a portion of a nucleus pulposus. A preferred dose protocol is one involving the maximal dose or dose frequency that avoids significant undesirable side effects. A total weekly dose depends on the type and activity of the compound being used. For example, such a dose is at least about 0.05 μg/kg body weight, or at least about 0.2 μg/kg, or at least about 0.5 μg/kg, or at least about 1 μg/kg, or at least about 10 μg/kg, or at least about 100 μg/kg, or at least about 0.2 mg/kg, or at least about 1.0 mg/kg, or at least about 2.0 mg/kg, or at least about 10 mg/kg, or at least about 25 mg/kg, or at least about 50 mg/kg (see, e.g., Yang, et al. New Engl. J. Med. 349:427-434, 2003; or Herold, et al. New Engl. J. Med. 346:1692-1698, 2002).

An effective amount of a modulator of GDF-6 signaling for a particular patient may vary depending on factors such as the condition being treated, the overall health of the patient, the method route and dose of administration and the severity of side affects, see, e.g., Maynard, et al. (1996) A Handbook of SOPs for Good Clinical Practice, Interpharm Press, Boca Raton, Fla.; or Dent (2001) Good Laboratory and Good Clinical Practice, Urch Publ., London, UK.

Determination of the appropriate dose is made by a clinician, e.g., using parameters or factors known or suspected in the art to affect treatment or predicted to affect treatment. Generally, the dose begins with an amount somewhat less than the optimum dose and is increased by small increments thereafter until the desired or optimum effect is achieved relative to any negative side effects. Important diagnostic measures include those of symptoms of the disease and/or disorder being treated. Preferably, a compound that will be used is derived from or adapted for use in the same species as the subject targeted for treatment, thereby minimizing a humoral response to the reagent.

An effective amount of therapeutic will decrease disease symptoms, for example, as described supra, typically by at least about 10%; usually by at least about 20%; preferably at least about 30%; more preferably at least about 40%, and more preferably by at least about 50%.

The present invention is described further in the following non-limiting examples.

### EXAMPLE 1

### Mutations in GDF-6 are Associated With Aberrant IVD Development

1.1 Materials and Methods

Subjects

A large family of subjects suffering from an autosomal dominant form of Klippel-Feil Syndrome (KFS) was identified (designated KF2-01). The affected subjects had large block fusions of vertebrae within in the spine, or isolated cervical fusions, or fusions of cervical, thoracic and lumbar vertebrae, indicating that these subjects had aberrant IVD development.

FISH Chromosome Inversion Analysis

Cytogenetic analyses of the KF2-01 family indicated the presence of inversion breakpoints located on 8q22.2 and 8q23.316. From the National Center for Biotechnology Information (NCBI) database a contiguous array of bacterial artificial chromosome (BAC) clones from the genomic regions flanking the inversion were selected. FISH chromosome analysis was performed as follows: metaphase spreads were prepared from PHA-stimulated lymphocytes, cultured at 37° C. for 72 hr. High resolution analysis of elongated chromosomes was carried out using dual-colour fluorescence. Total DNA isolated from BAC clones (Invitrogen, Australia) was nick-translated using fluorescent labelled dUTP (spectrum green and spectrum red, Vysis Inc.). Hybridization to metaphase chromosomes was performed essentially as described in Pinkel et al., Proc. Natl Acad. Sci. USA, 83: 2934-2938, 1986. For each slide, 400 ng of fluorescent labelled DNA was used. Before hybridization, the labelled probe was annealed with a 400-fold excess amount of Cot-DNA (Immunodiagnostics Pty Ltd, Australia) at 37° C. for 45 min. Chromosomes were counterstained with DAPI or propidium iodide diluted in anti-fade solution pH8. Fifty metaphases were analysed for each hybridization. Images were captured and merged using an Imstar digital FISH imaging system (Immunodiagnostics, Australia).

Inversion Breakpoint Analysis

The FISH screening strategy was based on the principle that any BAC clone/probe which spanned a breakpoint would display a split / dual hybridization signal. Two BAC clones (AC026561 and AC012238) were identified from either end of the inversion that gave split signals. To clone the proximal inversion breakpoint a set of forward PCR primers were designed at 5-kb intervals across the region of interest in each breakpoint BAC (AC026561 and AC012238), respectively. The forward primers from both BACs were combined to yield a unique PCR amplification product from patient DNA which contained the proximal inversion breakpoint. Primers used to amplify the proximal inversion breakpoint from affected KF2-01 family members were: 1F primer 5′- ATCCCTTAGTTGAACACAAAAAGCACAAGC-3′ (from BAC AC026561) (SEQ ID NO: 10) and the 2F primer 5′-TTCTATAAAGATCATCCATGCTAAACACTG-3′ (from BAC AC012238) (SEQ ID NO: 11). To clone the distal inversion breakpoint this PCR protocol was repeated using a mixed reverse primer set comprising: 1R primer 5′-TGTATGAGAGTTTTGGTGGTTCCACATC-3′ (SEQ ID NO: 12), and 2R 5′-GATAAGGACTGAGATATGCCCTGGT-3′ (SEQ ID NO: 13).

Long-range breakpoint PCR was performed in a 25 μl reaction mixture containing 50 ng of genomic DNA, 0.2 μM of each primer, 200 μM dNTPs, and 1 U Elongase enzyme (Life Technologies). An initial 3 min denaturation step at 95° C.; 32 cycles of denaturation at 95° C. for 30 s, annealing at 60° C. for 30 s and extension at 72° C. for 7 min; followed by a final extension at 68° C. for 3 min. PCR products were purified using QIAquick Spin PCR purification kit (QIAGEN) before sequencing.

DNA Sequencing

DNA sequencing was performed using the ABI Big Dye Terminator version 3.1 cycle sequencing kit essentially according to manufacturer's instructions (i.e. 5 ng (5 μL) purified PCR amplicon, 4 μL reaction pre-mix, 2 μL 5× sequencing buffer, 3.2 pmol (2 μL) appropriate primer and 7 μL deionized water were added in a 96-well microtiter plate. The plate was transferred to a PCR thermocycler (MJ Research PTC-200) and cycled at: 96° C. for 1 min; 25 cycles at 96° C. for 10 s, 50° C. for 5 s and 60° C. for 4 min. Sequencing products were purified using ABI Centri-Sep spin columns. Resuspended samples were resolved on an ABI 377 DNA sequencer, essentially according to the manufacturer's instructions and sequences analyzed using the BioEdit biological sequence alignment editor (v 5.0.9.1; Tom Hall, Isis Pharmaceuticals).

Mutation Screening

The 2 exons of GDF6 were screened by automated sequencing, including at least 50 by into the intron boundaries. The transcription start codon resides in exon 1. Primers were designed using the Primer3 program and were synthesised by Invitrogen Australia. PCR was performed in a 25 μL reaction mixture containing 50 ng genomic DNA, 0.2 μM of each primer, 200 hd μM dNTPs in 1×PCR buffer with 5% DMSO and 0.25 U Taq polymerase (Promega). Before thermal cycling, samples were denatured at 95° C. for 4 min followed by five touch down cycles of 95° C. for 40 s denaturation, 65° C. for 40 s annealing and 72° C. for 50 s extension; then 28 cycles of 95° C. for 40 s denaturation, 60° C. for 40 s annealing and 72° C. for 50 s extension with a final extension at 72° C. for 10 min (Corbett Research CG1-96). PCR products were resolved by electrophoresis in 1.4% agarose gels and purified using the Promega Wizard gel purification system before bi-directional sequencing.

Protein Sequence Alignment

Sequence alignments were carried out using ClustalW software. Proteins from aligned species included Homo sapiens, Macaca mulatta, Mus musculus, Rattus norvegicus, Xenopus laevis, Danio rerio and Tetraodon nigroviridis. GDF6 secondary structure was predicted for GDF6 using PROF 19. The cysteine knot was prepared in comparison with GDF520 using PyMOL graphics system (Delano Scientific USA).

Analysis of Conserved Noncoding Sequences (CNSs)

To determine if any conserved DNA elements were located in the breakpoint region a comparative analysis of genomic sequences from multiple species was performed. The breakpoint occurs between GDF6 (630-kb 3′) and C8orf37 (hypothetical protein LOC157657) (180-kb 5′). The genomic sequence in the interval between GDF6 and C8orf37 were extracted from the Ensemb1 and NCBI GenBank databases for human, chimpanzee, dog, mouse, rat, chicken, and opossum and analysis for CNSs using VISTA software with the human sequence as the reference sequence (Frazer et al., Nucl. Acid Res., 32: W273-279, 2004).

A CNS was defined to be 100 bp ungapped alignment with at least 70% identity. The human gene annotation was obtained from the Ensembl database and the repeat information was obtained from RepeatMasker.

Tissue Collection

The nucleus pulposus (NP) and out region of the annulus fibrosus (AF) were collected fresh from 1 subject (age 16) undergoing a lumbar total disc replacement surgery.

One normal Spraugue-Dawley male rat weighting 380 g was anesthetised and humanly sacrificed before the lumbar spinal disc and vertebrae were immediately dissected.

Immunohistochemistry of Human and Rat Vertebrae

All tissue specimens were immediately fixed in 10% neutral buffered formalin for 5 hour and washed with PBS followed by embedding in paraffin. Animal disc tissue was decalcified (RDO solution, Lomb Scientific Australia) for 24 h. 4 μM mid-sagittal serial sections were cut and mounted on Super Plus slides (Lomb Scientific). Hematoxylin-eosin (H&E) staining was performed for general histological examinations. For Alcian blue staining (proteoglycan), and Safranin-O staining (newly deposited matrix), serial sections were de-waxed in xylene and re-hydrated through graded ethanol and stained in Alcian blue solution (1% WN, pH 4.2) or Safranin-O for 15 min. Nuclei were counter stained with nuclear red solution.

For immunohistochemical staining, slides were deparaffinized and hydrated through graded ethanol and equilibrated in Tris-HCl (pH 7.6) buffer. Antigen retrieval was achieved using DAKA Target Retrieval Solution. Endogenous peroxidase activity was quenched with 3% (v/v) H₂O₂ and nonspecific binding blocked with 10% skimmed milk powder in Tris buffered HCl. Primary rabbit anti-human GDF-6 polyclonal (GDF6) (1:500 dilution, Alpha Diagnostic Int.) was incubated on the slides for 1 hour at room temperature, washed and treated with MULTILINK solution (DAKO, Australia) followed by incubation with streptavidin conjugated peroxidase. The sections were visualized with 3,3′-diaminobenzidine hydrochloride solution (DAB, DAKO) and counterstained with Haematoxylin. Negative controls were treated in a similar manner.

1.2 Results

Inversion Breakpoint Localized Within GDF6 Locus

To identify the location of breakpoints on 8q22.2 and 8q23.316 in KFS subjects FISH chromosome analysis using chromosome 8 specific BAC probes was performed. Two BACs (AC026561 and AC012238) were identified that spanned the respective breakpoints which gave unique split hybridization signals on chromosome 8q (confirmed in twenty metaphases). Breakpoint specific PCR screening verified cosegregation of the inversion with the disease phenotype in twenty affected and four unaffected KF2-01 family members. Breakpoint PCR amplicons were sequenced and the proximal and distal breakpoints identified at nucleotide position 96544749 and 116078713, respectively, on chromosome 8q. The full length of the inverted segment was 19,533,963 bp. No additional rearrangement or DNA loss was associated with either breakpoint. Both inversion breakpoints were localized within extensive intergenic regions significant distances from neighbouring genes.

The distal breakpoint occurred within an intergenic region 1.6 Mb 5′ from CSMD3 and 400 kb 3′ from the TRPS1 human disease gene. CSMD3 is expressed predominantly in fetal brain24, and TRPS1 mutations are causative in trichorhinophalangeal syndrome type I. The TRPS1 gene was not disrupted by the inversion. TRPS1 patients with previously characterized deletions in this same region 3′ of the TRPS1 gene did not present with KFS-like phenotypes (Ludecke et al., Am. J. Hum. Genet., 68: 81-91, 2001).

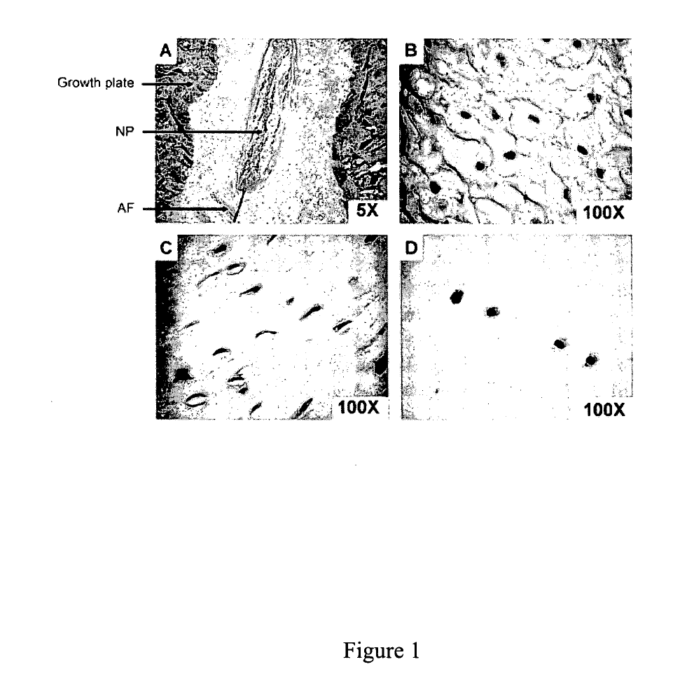

The proximal KF2-01 inversion breakpoint also occurred within an intergenic region 180 kb 5′ of transcript C8orf37 (hypothetical protein LOC157657) and 630 kb 3′ of GDF6. C8orf37 is a transcription unit of unknown function and GDF6, a member of the BMP family of secreted signalling molecules, is implicated in skeletal development. This genomic region between GDF6 and C8orf37 is known to harbor GDF6 long range enhancer elements (Mortlock et al., Genome Res., 13: 2069-2081, 2003). Regions rich in conserved non-coding sequences adjacent to the breakpoint were identified. With the exception of GDF6, no other genes located adjacent to the KF2-01 inversion breakpoints have recognised developmental or biological roles or known expression patterns which overlap with the KF2-01 familial phenotype. GDF6 expression was also observed within nucleus pulposus cells of both rat and human adult IVDs (FIG. 1). As a strong candidate for KFS, GDF6 was subsequently screened for mutations in our large cohort of patients.

GDF6 Missense Mutations in KFS Patients

GDF6 coding regions and associated exon splice sites were sequenced in 105 patients with de novo or inherited cases of KFS. Two new polymorphisms were identified in both the KFS and control populations screened; c.506+28C>A and c.1036G>C (p.SER312SER) at a frequency of approximately 4% of the population tested. Two different missense mutations were identified in three unrelated cases of KFS. In each case, the mutation was not detected in 174 controls (i.e. 348 chromosomes tested) giving ˜95% power to distinguish a normal sequence variant from a mutation (Collins et al., Am. J. Hum. Genet., 71: 1251-1252, 2002. None of the base substitutions found was present in the NCBI dbSNP database.

GDF6A249E Missense Variant

A heterozygous (c.846C>A) missense mutation segregating with KFS patterns of vertebral fusion (always inclusive of the C2-3 fusion) was also identified. The mutation (c.846C>A) in exon 2 resulted in the substitution of glutamic acid for alanine 249 (GDF6A249E) within the GDF6 prodomain. The GDF6A249E missense variant segregated with the KFS phenotype in the family and was absent from unaffected family members and ethnically matched normal controls.

Recurrent GDF6L289P Missense Variant

A recurrent heterozygous missense mutation c.966T>C was identified in two unrelated patients with sporadic KFS. Sequencing identified a recurrent missense mutation (c.966T>C) in exon 2 which resulted in the substitution of leucine for proline at position 289 GDF6L289P.

The missense mutations identified in the present study both cause amino acid changes in a region of GDF6 that is predicted to be required for GDF6 homodimerization and/or heterodimerization and, as a consequence GDF-6 signaling. Accordingly, these results indicate that disruption of GDF-6 signaling results in KFS syndrome, and, as a consequence aberrant IVD development and/or maintenance. This is supported by the persistence of GDF6 expression in the nucleus pulposus cells of the adult vertebral disc (FIG. 1) may indicate an extended role for GDF6 in disc maintenance.

### EXAMPLE 2

### Over Expression of MSX-1 or MSX-2 in IVD Cells Induces Collagen Formation and Extracellular Matrix Formation

2.1 Materials and Methods

Nucleus Pulposus Cultures

Nucleus pulposus tissue were visually separated from annulus fibrosus and aseptically procured from a cadaveric sheep spine (2 years of age) into sterile saline. Tissues were cut into ˜1 mm² pieces and then digested overnight with 0.025% collagenase solution in a shaking incubator at 37° C. Isolated cells were grown in 10% fetal calf serum (FCS) with 1% antibiotics (P/S/F) in DMEM (Invitrogen, Carlsbad, Calif.) culture media until confluency.

Lipofectamine Transfection

Transfection was performed with either 80 ng or 240 ng of an expression vector including a nucleic acid encoding MSX1 or MSX2 with an empty expression vector premixed with 18 μl Lipofectamine 2000 in Opti-MEM (Invitrogen) using 6-well plates (3×10⁵ cells per well for six wells), essentially according to manufacturer's instructions. At two days post-transfection, cells were selected and maintained with 600 μg G-418 Sulfate (Invitrogen) per ml of culture media.

Relative quantitation of MSX1 or 2 activity was determined using a MSX1/2 ELISA ^{PLUS} Kit essentially according to manufacturer's instructions. Relative activity was determined by the following calculation:

[((Absₛₐₘₚₗₑ−Abs_{heat inactivated sample})/Abs_{internal standard})/((Abs_{positive control}−Abs_{lysis buffer})/Abs_{internal standard})]×100.

Cell Viability Assay

Cell survival was measured with MTS Cell Proliferation Assay kit (Promega, Madison, Wis.) using cells (1×10⁴) /well plated in 96 well plates. Assays were performed as specified by the manufacturer where only viable cells are able to metabolically reduce tetrazolium salts to formazan salts, detected directly on a spectrophotometer at 490 nm

Collagen Synthesis

Collagen synthesis was assessed by [3H]-proline incorporation. L- [2,3-3H] Proline (Perkin Elmer, Sydney Australia) was added to culture medium at a concentration of 2 μCi/200 μL media. Cells were incubated for 24 hours. Cells were then harvested after washing with 95% ethanol and PBS. Radioactivity of cells was counted in a liquid scintillation counter.

Proteoglycan Synthesis

Proteoglycan synthesis was assessed by [35S]-sulfate incorporation. [35S]-sulfate (Perkin Elmer, Sydney Australia) was added to cell cultures at 2 μCi per well and allowed to incubate for 24 hours. Cells washed with 95% ethanol and PBS and harvested. Radioactivity of the cells was counted in a liquid scintillation counter as a representative of proteoglycan synthesis.

2.2 Results

Anulus fibrosus cells or nucleus pulposus cells were isolated from sheep IVDs and cultured for three passages. Following this period cells were transfected with an expression vector expressing MSX-1 or MSX-2 under control of the CMV promoter or a control vector (empty expression vector). Cells were transfected with two different concentrations of expression vector (i.e., 80 ng or 240 ng).

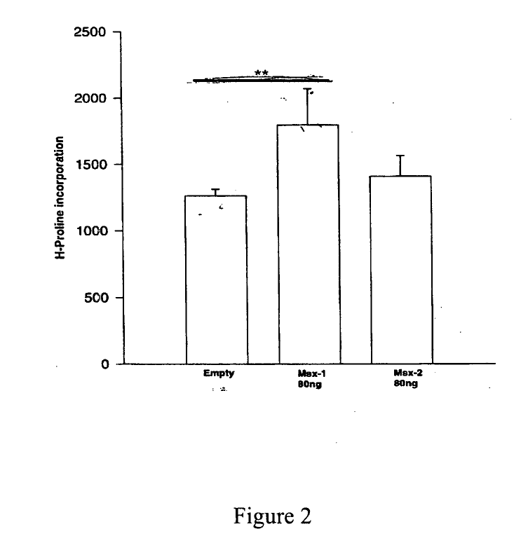

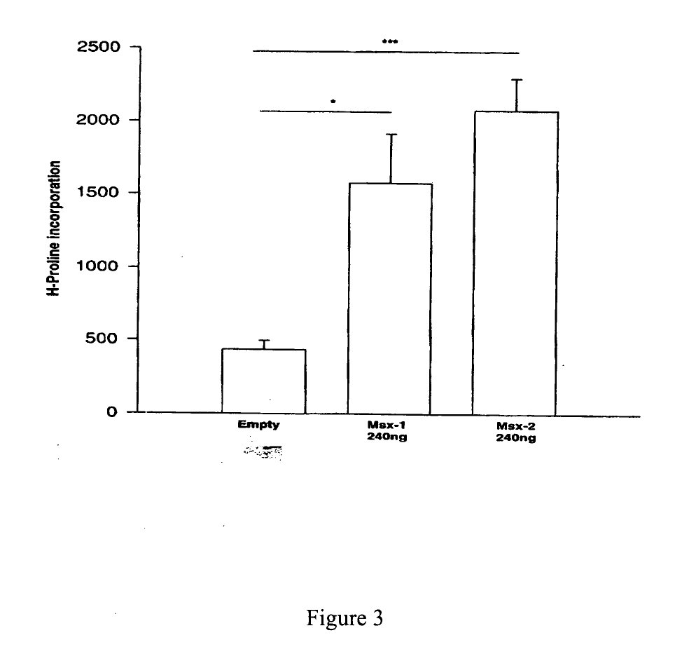

Following a suitable period for the introduced nucleic acids to be expressed, cells were assayed for collagen production, by determining the level of incorporation of H-proline into cells. As shown in FIG. 2, at the 80 ng dosage level, MSX-1 induced a significant increase in collagen synthesis in anulus fibrosus cells. At the 240 ng dosage, both MSX-1 and MSX-2 induced a significant increase in collagen synthesis in annulus fibrosus cells compared to control cells (FIG. 3).

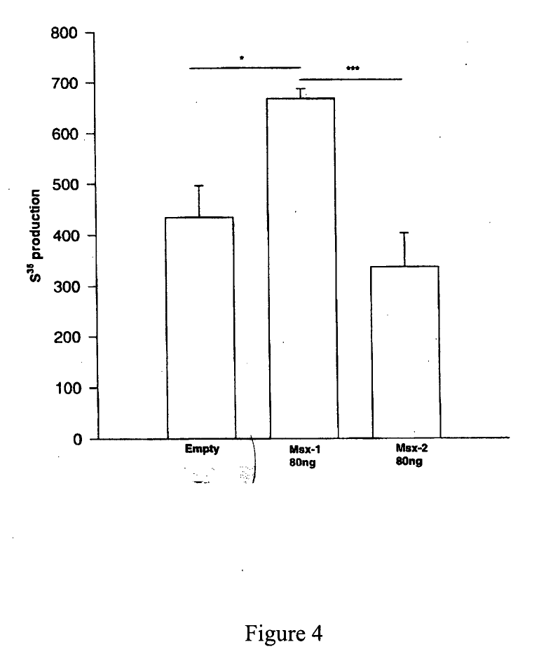

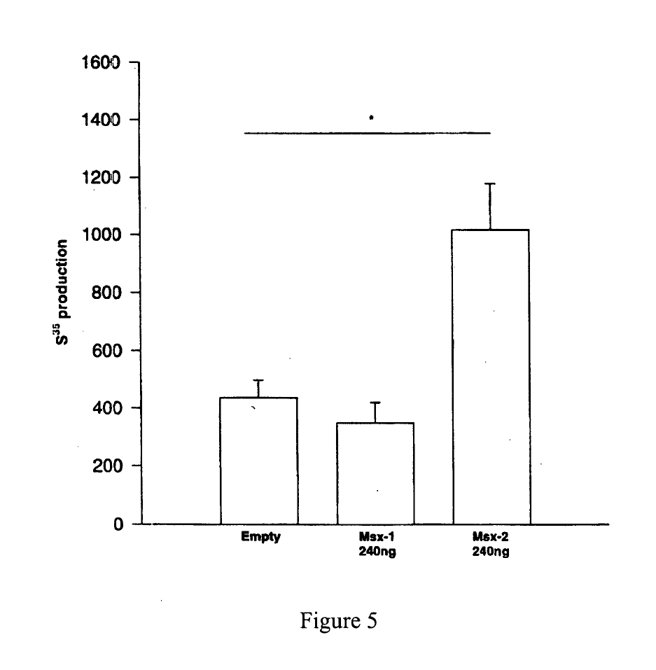

Cells were also assayed to determine the level of extracellular matrix production, be determining the level of ³⁵S incorporation into a culture. As shown in FIG. 4, 80 ng of nucleic acid encoding MSX-1 significantly increased extracellular matrix production compared to control cells. Moreover, 240 ng of nucleic acid encoding MSX-2 significantly increased the level of extracellular matrix production above control cells (FIG. 5).

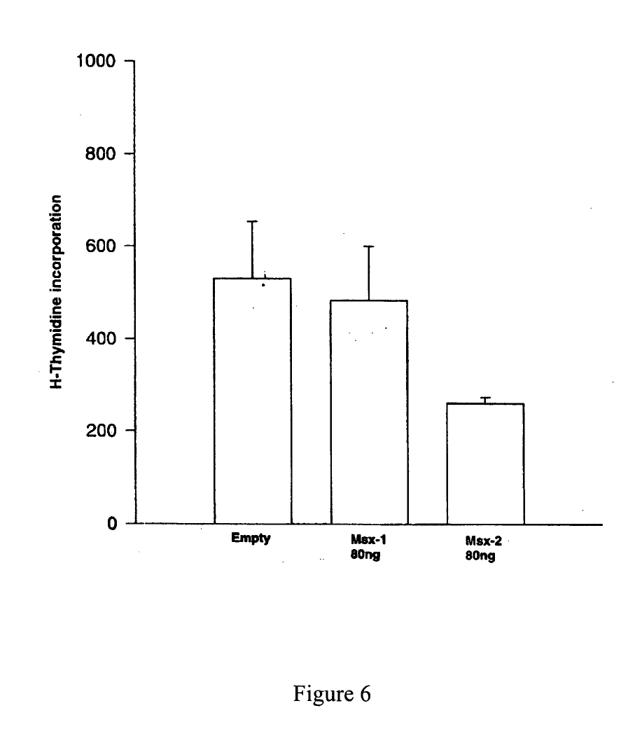

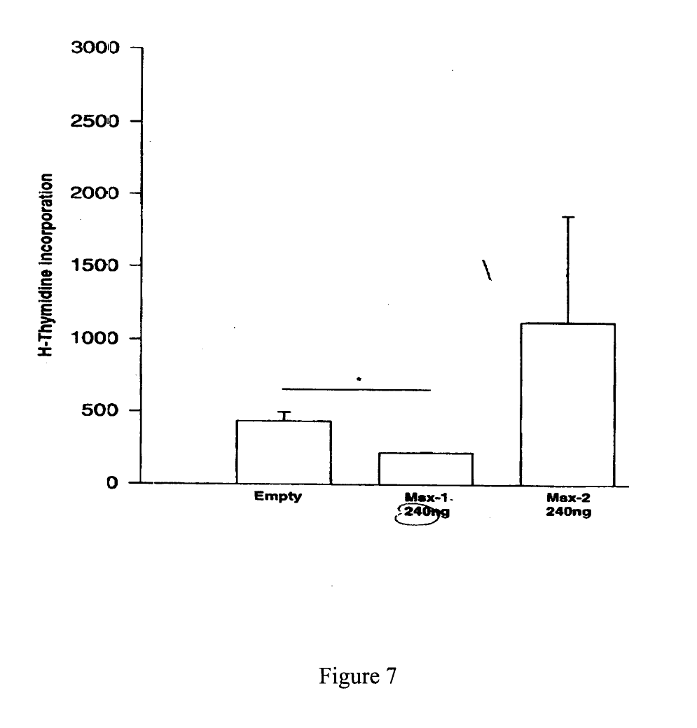

As shown in FIGS. 6 and 7, ectopic expression of MSX-1 or MSX-2 did not significantly alter the incorporation of H-thymidine by transformed cells, i.e., did not increase cell proliferation in annulus fibrosus cells.

These results indicate that MSX-1 and/or MSX-2 are capable of inducing changes in IVD cells associated with IVD regeneration.

### EXAMPLE 3

### Additional characterization of cells over expressing MSX1 or MSX2

3.1 Cell Cultures

Sheep nucleus pulposus cells are produced as described in Example 2

Human nucleus pulposus cultures are produced by collecting nucleus pulposus from eight subjects undergoing lumbar total disc replacement surgery (age: 48±16 years). All discs demonstrate moderate signs of disc degeneration on MRI including decreased water content and a decrease in disc height. Discarded nucleus pulposus tissues are immediately subjected to 0.025% collagenase digestion overnight. Primary cultures are grown in a complete medium containing DMEM (Invitrogen, Carlsbad, Calif.) 10% fetal calf serum, 1% penicillin/streptomycin for 10-12 days to become confluent. Cells are subcultured at concentration of 1×1 0⁵/ml for 2-3 days before treatment. All experiments are completed using the second passages of cells.

Cells are transfected and analyzed essentially as described in Example 2.

3.2 Extracellular Matrix mRNA Detection

RNA extraction from pooled aliquots of six flasks for each of control and transfected cells is performed with RNeasy Mini Kit (Qiagen, Hilden, Germany) and concentrated with a vacuum centrifuge. RNA is digested with DNase I Amplification Grade (Invitrogen) prior to the ImProm-II™ Reverse Transcription System (Promega, Madison, Wis.) for the generation of cDNA using Oligo(dT)₁₅ primers and 6.3 mM MgCl₂ per reaction essentially in accordance with manufacturer's instructions.

Expression levels of collagen Type 1, Collagen Type 2, Aggrecan and GAPDH is determined using the following primers: Collagen Type-1: [Forward] AGACATCCCACCAATCACCT (SEQ ID NO: 14) [Reverse] AGATCACGTCATCGCACAAC (SEQ ID NO: 15); Collagen Type-2: [Forward] AACACTGCCAACGTCCAGATG (SEQ ID NO: 16); [Reverse] TCGTCCAGATAGGCAATGCTG (SEQ ID NO: 17); Aggrecan: [Forward] ACGTGATCCTCACGGCAAA (SEQ ID NO: 18); [Reverse] GTGAAAGGCTCCTCAGGTTCTG (SEQ ID NO: 19); GAPDH: [Forward] ACCCAGAAGACTGTGGATGG (SEQ ID NO: 20) [Reverse] AGAGGCAGGGATGATGTTCT (SEQ ID NO: 21). Real time reactions are performed in triplicates with Platinum® Syber® Green qPCR SuperMix UDG (Invitrogen) using a Rotor-Gene Thermal cycler (Corbett Research, Sydney, Australia) programmed for: 50° C., 95° C. for 2 min hold each, 50 cycles of (94° C., 30 sec; 60° C., 30 sec with a 1° C. drop per cycle for the first five cycles; 73° C., 1 min). Gene expression of transfected cells relative to controls is analyzed using the relative expression software tool (REST®)[Pfaffl 02]. Statistical significance is determined by the pair wise fixed reallocation randomization test provided with the software.

3.3 Western Blot Analysis

The cells are harvested in lysis buffer containing a protease inhibitor cocktail (500 μg/ml AEBSF and 1 μg/l E-64, leupeptin, pepstatin-A at 2 μg/ml each). Protein (20-40 μg) is resolved on a 7.5% or 12% (v/v) SDS-PAGE gel. Proteins are transferred onto a PVDF membrane. Subsequently the membrane is probed with goat anti-Collagen-II polyclonal antibody (1:500, Santa Cruz Biotechnology), or anti-caspase-3 monoclonal antibody or anti-cleaved caspase-3 monoclonal antibody (1:1000 and 800, Cell Signalling technology) for 60 minutes. Membranes are then washed prior to addition of the corresponding secondary antibody conjugated with peroxidase (Chemicon, Temecula, Calif.) at a 1:1000 dilution for 30 minutes. A chemiluminescence detection system (Pierce) is then used for the visualisation of labeled proteins. Blots are stripped and re-probed with mouse anti-B-Actin monoclonal antibody (1:10000, Sigma) to ensure equal amounts of protein are loaded per lane. Visualized bands are semi-quantified by densitometry (Model GS-700/690, Bio-Rad, Hercules, Calif., USA).

3.4 Immunofluorescence Staining (Collagen-II and Aggrecan)

Immunofluorescence staining is performed after fixation with 4% paraformaldehyde of cells cultured on glass cover slips. Non-specific binding is then blocked with 5% normal donkey or sheep serum for 30 minutes. Primary goat anti-collagen type-II polyclonal (1:200) or mouse anti-human aggrecan monoclonal antibodies (1:150, Chemicon) are incubated on individual slides for 1 hour. Cells are repeatedly washed and secondary antibodies; donkey anti-goat or sheep anti-mouse IgG conjugated with FITC (Chemicon) is applied at 1:500 dilutions. Cells on mounted cover-slip are visualized using a fluorescence microscope (Leitz, Wetzlar). Negative controls are treated in a similar manner but with the omission of primary antibody and are consistently included in each experiment.

3.5 Alkaline Phosphatase Production

To ensure that MSX-1 or MSX-2 over expression results in the production of disc cells and not in the production of bone cells, Alkaline phosphatase (AP) activity is determined by lysing cells with 0.1% Triton X-100 in PBS buffer and lysates were then incubated for 30 minutes at 37° C. with the AP substrate, p-nitrophenylphosphate (Sigma-Aldrich) at 2.5 μg/ml. The levels of p-nitrophenol (PNP) production were measured by a spectrophotometer and concentrations were determined by comparison with a standard curve created with known amounts of p-nitrophenol. AP activity is expressed as nanomoles of PNP generated per microgram of total cellular protein per minute.

3.6 Proteoglycan Synthesis

Cell cultures were maintained in complete medium containing 10 μCi/ml of [³⁵S]-sulfate (Amersham Biosciences Corp., Australia) for 8 hours.

Proteoglycans were extracted from cells or medium with 4 M guanidinium hydrochloride (in 50 mmol sodium acetate pH 5.8 containing 0.1 M 6-amino-hexanoic-acid, 50 mmol benzamidine HCl, 10 mmol EDTA, and 5 mmol N-ethylmaleimide) at 4° C. for 24 hours. Total synthesis was determined by combining radioisotope incorporation of both the cells and condition medium using a rapid filtration assay (essentially as described in Masuda et al., Anal Biochem, 217: 167-175, 1994). Proteoglycans (PG) were precipitated by alcian blue (Sigma). The newly synthesized proteoglycans was detected by using a [beta]-liquid scintillation counter. Rates of [³⁵S]-incorporation were expressed as nmols [³⁵S]- incorporated/μg DNA.

### EXAMPLE 4

### In Vivo Models of IVD Degeneration

4.1 Rabbit Model

Adolescent New Zealand white rabbits (weighing 3.5-4 kg) are anaesthetized and two non-contiguous discs (L2/3 and L4/5) are punctured with an 18G needle using a left retroperitoneal approach, to induce disc degeneration. Four weeks later, eight rabbits were sacrificed for baseline assessments of the annular puncture.

4.2 Sheep Model

Sheep are fasted for 24 hours prior to surgery and then anaesthetized. A lateral plain X-ray is taken to verify normal lumbar spine anatomy. A skin incision is made on the left side immediately anterior to the transverse processes of the spine and the lumbar spine exposed by blunt dissection using an anterior muscle-splitting technique. Sheep receive controlled annular lesions in their L1-L2, L3-L4 and L5-L6 discs by incision through the left anterolateral annulus fibrosus parallel and adjacent to the cranial endplate using a scalpel blade to create a lesion measuring approximately 4 mm long and approximately 5 mm deep. The intervening lumbar discs (L2-L3, L4-L5) are not incised. A non-operated disc remains between treated discs to allow for adequate anchorage of FSUs in subsequent mechanical testing. A wire suture is used to identify the craniad operated level for later identification purposes both in X-rays and for morphological identification.

Three months after induction of the annular lesions the sheep are killed and the lumbar spines, are radiographed to evaluate disc calcification, excised and processed for biomechanical and histochemical analyses, and, after the biomechanical testing the same discs zonally dissected for compositional analyses.

### EXAMPLE 5

### Treatment of IVD Degeneration With Recombinant GDF-6

5.1 Administration of GDF-6 to Animals

Recombinant human GDF-6 is obtained from a commercial source, such as, for example, US Biological, MA, USA.

Animal models as described in Example 4 are treated with recombinant GDF-6. For example, incised discs receive one of three therapies administered using a standard needle or essentially as described in Example 9 hereof, (I) no treatment, (II) lactose solution or (III) lactose containing GDF-6. In all animals the L3-L4 disc receives an annular lesion with no treatment. In one group of animals the L1-L2 discs are treated with lactose solution only and the L5-L6 disc are treated with lactose plus GDF-6. In another group of animals the treatments in the L1-L2 and L5-L6 discs are reversed to avoid any potential outcome bias associated with spinal level.

5.2 Radiological and MRI Assessments

Disc height is radiographically monitored biweekly from the day of administration of the above-treatment to 24 weeks post-administration. Intervertebral height is expressed as the disc height index (DHI) (Percent DHI (% DHI=(postoperative DHI/preoperative DHI)×100). At 4-, 8-, 12- and 24-weeks after injection, an MRI of the spinal column is taken to grade the level of degeneration based on modified Thompson grade (MRI, 1=normal, 4 severely degenerated) (Masuda et al., Spine 30:5, 2004)

5.3 Proteoglycan and Collagen Contents of Disc Tissues

Samples of annulus fibrosus and nucleus pulposis are diced over ice and representative portions of each tissue zone of known wet weight is freeze dried to a constant weight. The difference between the starting and final weights of the tissues is indicative of water content of the tissue. Triplicate portions (1-2 mg) of the dried tissues are hydrolyzed in 6M HCl at 110° C. for 16 h and aliquots of the neutralized digests assayed for hydroxyproline as a measure of the tissue collagen content (essentially as described in Melrose et al., J Orthop Res 10:665-676, 1992; and Melrose et al., Matrix 14:61-75, 1994. Triplicate portions of dried tissues are digested with papain and aliquots of the solubilized tissue assayed for sulphated glycosaminoglycan using the metachromatic dye 1,9-dimethylmethylene blue as a measure of tissue proteoglycan (see Melrose et al., 1992 and 1994, supra).

5.4 Histochemical and Immunohistochemical Analyses

Spinal motion segments that are designated for histochemical analysis are isolated by cutting through the cranial and caudal vertebral bodies close to the cartilaginous endplates. Entire disc specimens including the adjacent vertebral body segments are fixed en bloc in either 10% neutral buffered formalin or Histochoice™ for 56 h and decalcified in several changes of 10% formic acid in 5% NBF for 2 weeks with constant agitation until complete decalcification is confirmed using a Faxitron HP43855A X-ray cabinet (Hewlett Packard, McMinnville, USA).

Sagittal slices (5 mm thick) of the decalcified disc-vertebral body specimens are dehydrated through graded ethanol solutions by standard histological methods and embedded in paraffin wax. Paraffin sections 4 μm thick are prepared for histochemical staining and mounted on Superfrost Plus glass microscope slides (Menzel-Glaser) and dried.

Sections are deparaffinized in xylene and rehydrated through graded ethanol washes (100-70% v/v) to water.

Sections from all blocks are stained with haematoxylin and eosin. These sections are examined by a histopathologist who compares the histological characteristics of those levels that receive annular incision only with those that are incised and receive GDF-6. A four-point semi-quantitative grading system is used to assess the microscopic features. Collagen architecture is also examined in sections stained with Masson's trichrome and picro-sirius red using polarized light microscopy.

For immunohistochemistry endogenous peroxidase activity is blocked by incubating the tissue sections with 3% H₂O₂. Tissue sections are then treated with combinations of chondroitinase ABC (0.25 U/ml) in 20 mM Tris-acetate buffer pH 8.0 for 1 h at 37° C., bovine testicular hyaluronidase 1000 U/ml for 1 h at 37° C. in phosphate buffer pH 5.0, followed by washes in 20 mM Tris-HCl pH 7.2 0.5M NaCl (TBS) or proteinase-K (DAKO S3020) for 6 min at room temperature to expose antigenic epitopes. The tissues are then blocked for 1 h in 20% normal swine serum and probed with a number of primary antibodies to large and small proteoglycans and collagens, Aggrecan, Perlecan, Versican, Decorin, Biglycan, Fibromodulin, Collagen Type I, Collagen Type II, Collagen Type IV, Collagen Type VI and Collagen Type X. Negative control sections are also processed either omitting primary antibody or substituting an irrelevant isotype matched primary antibody for the authentic primary antibody of interest. Horseradish peroxidase or alkaline phosphatase conjugated secondary antibodies are used for detection using 0.05% 3,3′-diaminobenzidene dihydrochloride and 0.03% H₂O₂ in TBS or Nova RED substrates. The stained slides are examined by bright-field microscopy and photographed using a Leica MPS 60 photomicroscope digital camera system.

5.5 Biomechanical Assessment of Spinal Motion Segments

Non-destructive biomechanical range of motion (ROM) analysis is conducted on each functional spinal unit (FSU) in various planes of motion (flexion-extension, lateral bending, compression and torsion). Each FSU comprises two adjacent vertebrae, the intervening disc and associated ligaments.

Four FSUs are tested: non-operated control levels; levels that are incised; levels that are incised and treated with GDF-6 and carrier and levels that are incised and treated with carrier alone. Each FSU is mounted in two aluminum alloy cups and secured with cold cure dental cement. Care is taken to ensure that the IVD is aligned with the cups. Prior to the commencement of testing each FSU is preloaded to a constant until a reproducible state of hydration is achieved. This constant stress is used as the baseline prior to each test. The constant stress simulates relaxed standing and is based on in-vivo measurement of intradiscal pressure (Wilke H-J et al., Spine 24:755-62, 1999). A torsional load and flexion-extension, lateral bending load is applied over 10 cycles whilst under a constant axial load. A cyclic axial load is applied to investigate the axial compression response of the IVD.

### EXAMPLE 6

### Intracellular Delivery of MSX-1 and/or MSX-2

6.1 Peptides

MSX-1 or MSX-2 polypeptide fused to a HIV-1 tat protein transduction domain and a hexa-histidine tag is produced by recombinant means. As a control a beta galactosidase protein fused to a HIV-1 tat protein transduction domain and a hexahistidine tag is produced. Recombinant protein is isolated using a nickel-NTA column.

6.2 Cells

Sprague-Dawley rats aged 11 months are euthanized and IVD tissue from the lumbar spine and tail harvested under sterile conditions. Annulus fibrosus and nucleus pulposus are separately dissected and diced. The IVD tissue is placed in Dulbecco's modified Eagle's medium and Ham's F12 medium (DMEM/F-12; GIBCO BRL, Grand Island, N.Y., U.S.A.) containing 100 unit/ml penicillin and 100 mg/ml streptomycin. The IVD tissue is treated with 0.2% pronase (Sigma Chemical, St. Louis, Mo., U.S.A.) in the medium for 1 hour at 37° C. and then treated with 0.025% collagenase (Sigma Chemical, St. Louis, Mo., U.S.A.) for 6 hours at 37° C. Isolated cells are washed and filtered through a 70 mm mesh (Falcon, Franklin Lakes, N.J., U.S.A.) into 75 cm² flasks with 12 ml DMEM/F-12 medium containing 10% fetal bovine serum (FBS), 100 unit/ml penicillin, 100 mg/ml streptomycin, 2 mM L-glutamine and 50 mg/ml ascorbate. The cells are grown at 37° C. in 5% CO₂ with humidification. The culture media is changed every 2 days for approximately 8 days.

When the primary culture of IVD cells become confluent, the cells are sub-cultured into 6-well plates at 400,000 cells per well. Three days later, the cells are treated with either the MSX-1 fusion protein or a MSX-2 fusion protein or both fusion proteins or the LacZ fusion protein. Cell number is determined at day 0 by counting a control well using a hemocytometer. Cells are maintained in the presence of the peptide for two weeks. The medium is changed every 3 days during the experiment.

The sulfated-glycosaminoglycan (sGAG) content of the culture media is assayed using the 1,9-dimethylmethylene blue (DMMB) method. The culture media 2 ml is centrifuged (5000×G for 30 minutes) to concentrate the sGAG using the Centricon YM-50 centrifugal filter (Millipore Co., Bedford, Mass., U.S.A.). The sample solution (20 ml) is mixed gently with 200 ml DMMB dye solution in a 96-well microtiter plate, and the optical density (OD) was checked immediately at 520 nm wavelength filter. A standard curve is constructed using serial dilutions of chondroitin sulfate (Sigma Chemical, St. Louis, Mo., U.S.A.). Total sGAG in the media is normalized by DNA content and presented as a ratio to the untreated control.

The cell number is determined by the DNA content of each well, and DNA content is measured with a Hoechst dye 33258 (Polysciences, Warrington, Pa., U.S.A.) method. Cultured cells are removed from the plate by exposure to papain (10 units/ml). Cells are pelleted and incubated at 60° C. for 3 hours. A twenty microliter aliquot of the papain digest is mixed with 200 ml of Hoechst dye 33258 solution in a 96-well fluoroplate. Emission and excitation spectra are measured in Luminescence Spectrometer LS 50B (Perkin-Elmer, Wellesly, Mass., U.S.A.) at 456 nm and 365 nm, respectively. Standard curves are generated at the time of each measurement using known concentrations of calf thymus DNA (Sigma Chemical, St. Louis, Mo., U.S.A.).

Recombinant peptides are administered to an animal model (e.g., as described in Example 4) using a standard needle or as described in Example 9 hereof. The effect of the peptides on the animals is determined essentially as described in Example 5.

### EXAMPLE 7

### Treatment of IVD Degeneration with Adenovirus Expressing MSX-1 or MSX-2

7.1 Adenoviral Constructs

Recombinant type 5 human adenoviral vectors with complete deletion of the E1A and E1B regions and a partial deletion of the E3 region of the viral genome are used in this study (Bett et al., Proc Natl Acad Sci USA 91: 8802-8806, 1994). A therapeutic vector contains a cDNA encoding MSX-1 or MSX-2 gene under control of the cytomegalovirus promoter (AD-MSX) at a concentration of 5×10¹² pfu/ml. Control adenoviral vector contains the beta-galactosidase gene under control of the cytomegalovirus promoter (Ad-beta-gal), also at a concentration of 5×10¹² pfu/ml.

7.2 Administration of Ad-GDF-6 and Ad-beta-gal

Rabbits treated as described in Example 4 are anesthetized. Viral solution comprising therapeutic vector or control vector (7.5

μl of a solution comprising 3.75×10¹⁰ pfu) as described above under section 7.1, is administered to a punctured disc that has been induced to undergo disc degeneration, or alternatively, a control disc. Therapeutic vector is administered into one disc in each animal and control vector is administered into a separate disc in each animal. Administration is achieved using a standard needle (e.g., a 19-gauge needle and a Hamilton microsyringe) or essentially as described in Example 9 hereof.

Animals are analyzed essentially as described in Example 5.

### EXAMPLE 8

### Treatment using Stem Cells expressing GDF-6 and/or MSX-1 and/or MSX-2

8.1 Expression Constructs

Nucleic acid encoding GDF-6 or MSX-1 or MSX-2 under control of a CMV promoter is inserted into an HIV-1-based self-inactivating (SIN) lentiviral vector (pHRSINcPPT-SEW). As a control, a vector expressing the eGFP reporter gene under the control of the spleen focus-forming virus (SFFV) LTR is used.

8.2 Isolation, Purification and Expansion of Mesenchymal Stem Cells (MSCs)

Bone marrow cells are collected by flushing the femurs, tibias and iliac crests from New Zealand white rabbits with PBS supplemented with 2% fetal bovine serum (FBS; Gibco, Paisley, UK). Red blood cell-depleted bone marrow mononuclear cells (BMMNCs) are plated at a density of 10⁶ cells/cm² in mesenchymal medium with mesenchymal supplements (Stem Cell Technologies, Vancouver, Canada), further supplemented with 100 IU/ml penicillin and 100 μg/m1 streptomycin (Gibco). Non-adherent cells are eliminated by a half medium change at day 3 and the whole medium is replaced weekly with fresh medium. The cells are grown for 2-3 weeks until attaining near confluence. The whole adherent fraction is then detached by trypsinization and re-plated using a 1:3 dilution factor. Subsequent passing and seeding of the cells is performed at a density of 5000 cells/cm². To enrich MSCs, adherent cells from passage 2 (P2) and 3 (P3) are stained with anti-CD45-CyChrome and CD11b-PE (BD Biosciences, Oxford, UK), or a combination of CD45 and biotin-conjugated lineage (Lin) cocktail antibodies (Stem Cell Technologies) followed by streptavidin-PE. The negative fraction from both cell surface antigens is sorted using the flow-activated sorter Vantage (Becton-Dickinson, Oxford, UK). Enriched MSC populations are cultured under the same conditions as described above.

8.3 In Vitro Lentivirus-Mediated Gene (eGFP) Transfer into MSCs

Transduction of MSCs is performed with the expression constructs as described above under section 8.1. For transduction, 1×10⁴ purified MSCs from passage 4 (purP4) are seeded into individual wells of a 12-well plate. The following day virus particles are added at multiplicity of infection (m.o.i.) of 5, 10, 30 or 50 and transduction is performed for 20 hours. Cells transduced with an eGFP expressing vector are harvested on day 1, 3 and 5 after virus removal and analyzed for eGFP expression by flow cytometry.

8.4 Treatment of Animals Using Transduced Stem Cells

Rabbits treated as described in Example 4 to induce IVD degeneration are treated with the transduced stem cells prepared as described above under section 8.3. Transduced stem cells (2×10⁶), that are obtained a few days post-viral removal to minimize further expansion, are administered directly into the treated IVD of the rabbits using a standard needle or as described in Example 9 hereof. Rabbits are analyzed essentially as described in Example 4 and 5 to determine the effect of stem cell infusion, i.e., re-population and repair of the degenerated IVD.

8.5 Tissue Processing and Immunohistochemistry

To determine the survival of stem cells, cells expressing eGFP in control administrations are stained for eGFP expressions. Tissues are fixed in 10% neutral buffered formaldehyde (NBF), embedded in paraffin and in some cases the other half of each tissue is cryo-embedded. Each embedded tissue is sectioned between 10 to 15 levels with a 70 to 100 μm gap between each one. Each level is serially sectioned at least 4 times. Sections (4 μm thick) are screened for the presence of eGFP either by staining with an eGFP antibody (Santa Cruz Biotechnology), or by direct visualization using a fluorescent microscope (Zeiss AxioVision2™, Zeiss, Welwyn Garden City, UK).

### EXAMPLE 9

### Use of a Device for the Delivery of GDF-6 Signaling Modulator to an IVD or a Region Adjacent or Surrounding an IVD in an Animal Model

Recombinant GDF-6 (Example 5) and/or recombinant MSX-1 (Example 6) and/or recombinant MSX-2 (Example 6) is administered to the animal models described in Example 4 hereof, essentially following the protocols described in Examples 5 and 6, with the exception that the GF6-modulatory composition is formulated in lactose solution and in a hydrogel or co-polymer for administration to an IVD or a region adjacent or surrounding an IVD using the device exemplified in FIGS. 8-18.

Referring to FIGS. 8-10, the delivery device 10 includes a delivery conduit 12 having a proximal end attachable to a source 14 of the compositions of the invention to be administered, the device 10 and the source 14 forming a system for the delivery of the compositions to an IVD or a region adjacent or surrounding an IVD in a subject in need thereof. The source 14 is, typically, a syringe for dispensing the compositions through the delivery conduit 12.

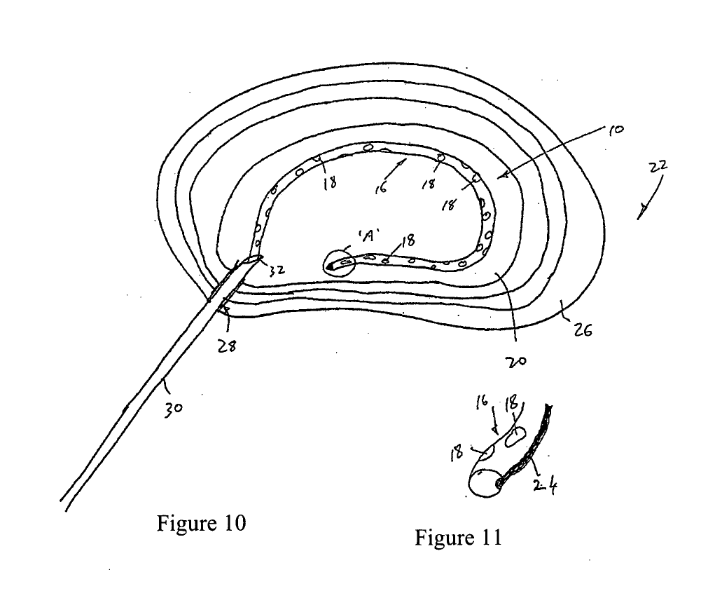

An emitter structure 16 is arranged at the distal end of the delivery conduit 12. The emitter structure 16 defines, as shown in greater detail in FIG. 10 of the drawings, a plurality of discharge apertures 18 arranged at longitudinally spaced intervals. The discharge apertures 18 are configured to effect uniform, diffuse distribution of a composition of the invention throughout an IVD nucleus 20 (FIG. 10) of an IVD 22. To effect the uniform, diffuse distribution of the composition, the apertures 18 closer to the distal end of the delivery conduit 12 are of smaller diameter than the apertures 18 distally arranged on the emitter structure 16. In such a fashion, there is a substantially uniform discharge of the agent through the apertures 18 of the emitter structure 16 to facilitate the diffuse distribution of the agent throughout the nucleus 20 of the IVD 22 or a region adjacent or surrounding an IVD.

The emitter structure may have a diameter in a range from about 0.1 mm to about 3.5 mm and may be formed of a reinforced, suitable plastics material, for example. The reinforcing may be in the form of bands (not shown) arranged at longitudinally spaced intervals to retain the emitter structure 16 in an open condition against pressure exerted by the tissue of the nucleus 20 in use.

The emitter structure 16 may be steerable to adopt a loop shape in the nucleus 20 as shown in FIGS. 8-10. To achieve this, the emitter structure 16 may have a steering wire or pull wire (not shown), for example, embedded in its wall. Manipulation of the steering wire is carried out by a clinician with the assistance of, for example, fluoroscopy, to ensure that the emitter structure 16 adopts a spread out configuration within the nucleus 20 of the IVD 22.

The emitter structure 16 may have alternative configurations such as a preformed guide wire instead of a steering wire such that the emitter structure 16 adopts a similar loop-shape, or it may be forked having a plurality of branches to effect distribution of the composition through the nucleus 20 of the IVD 22.

To introduce the emitter structure 16 into the nucleus 20 of the disc 22, an annulotomy is formed on an annulus 26 of the IVD 22. The annulotomy results in an access opening 28 being formed in the annulus 26 of the disc 22. A working cannula 30 is inserted percutaneously through the subject's skin in a minimally invasive manner. The working cannula 30 may also be used for performing the annulotomy on the annulus 26. Thus, a tip of the working cannula 30 is sharpened or beveled for effecting perforation of the annulus 26.

Once a tip 32 of the working cannula 30 has been inserted into the nucleus 20 through the opening 28, the emitter structure 16 of the delivery device 10 is extended through the end 32 of the working cannula 30 to adopt the position shown, for example, in FIG. 10 of the drawings and enabling a diffuse, substantially uniform distribution of the composition of the invention throughout the nucleus 20 to be effected.

Once delivery of the composition has been completed, a positive pressure is maintained in the envelope to inhibit back flow of the composition through the apertures into the interior of the emitter structure 16. This is done in one of a number of ways such as (a) having a non-return valve in each aperture; (b) maintaining a continuous pressure, for example, by a motorized pneumatic device (not shown) while withdrawing the emitter structure 16 into the working cannula 30 or (c) pumping air into the emitter structure 16 behind the agent.

Use of the device 10 targets the composition to the nucleus of an IVD and facilitates diffuse, substantially uniform distribution of a composition of the invention to the IVD such that the composition is more evenly distributed throughout the tissue, i.e., from the nucleus or to a region adjacent or surrounding an IVD.

### EXAMPLE 10

### Production of Recombinant Human GDF-6 and a Bioactive Fragment of GDF-6

10.1 Cell Transfections

Approximately 2.5×105 CHO cells maintained in serum free medium were transfected with an expression vector comprising full length GDF-6 cDNA fused to a FLAG tag or an expression vector comprising a cDNA encoding an active domain of GDF-6 fused to a FLAG tag (SEQ ID NO: 25) in 6-well plates using Lipofectamine 2000 (Invitrogen) according to manufacturer's instructions. Briefly, 12.5 μl of Lipofectamine 2000 was mixed with 5 μg vector in a total volume of 250 μL, 20 minutes before addition to CHO cell cultures. Lipofectamine/vector mixtures are then added to CHO cells and incubated for 5 hours. Proteins secreted into the supernatant were harvested and analyzed by Western blotting and Alkaline Phosphatase activity.

Western Blotting

Concentrated supernatants from one well of a 6-well plate were separated using polyaclylamide gel electrophoresis (PAGE) under either reducing conditions. Proteins were transferred to nitrocellulose membranes and detected with antisera specific for GDF-6 or for the FLAG tag, secondary antibodies and detection system.

Alkaline Phosphatase Assay

To test for GDF-6 activity, the ATDC5 chondroprogenitor mouse embryonal carcinoma cell line was used. ATDC5 100 μl of cells at a concentration of 1.5×10⁵ cells/ml were seeded per well onto a 96-well plate. After 24 hours, media was replaced with 100 μl/well with media containing 2 FCS. Cells were then incubated with GDF-6 or the active fragment whereby isolated GDF-6 or active fragment, or BMP7 stock, or GDF-6 (GDF-6 purchased stock) at a stock concentration of 1 μg/μl were diluted to a working concentration of 6.4 μg/ml. 200 μl of the working concentration was added to the first row of a 96-well plate for each ligand. The remaining rows received 200 ml of 1:2 serial dilutions of the working stock concentration. Cells were incubated for 72 hours washed twice with 100 μl of PBS then lysed directly with Alkaline Lysis Buffer I (0.1 M Glycine pH 9.6; 1% NP40; 1 mM MgCl₂ ; 1mM ZnCl₂). 10 ml of Alkaline Lysis Buffer 2 (0.1 M Glycine pH 9.6; 1mM MgCl₂; 1mM ZnCl₂) was pre-mixed with 1 ml of 20 mg/ml in water of alkaline phospatase substrate, p-nitrophenylphosphate (PNPP; Sigma-Aldrich). 100 μl of working solution of substrate PNPP was added to each well. Lysates were then incubated for 30 min to 1 hour at 37° C. or until the colour developed. The levels of p-nitrophenol (PNP) production were measured by a spectrophotometer by measuring absorbance at 405 nm in an ELISA plate reader, and concentrations were determined by comparison with a standard curve created with known amounts of p-nitrophenol. Alkaline phosphatase activity was expressed as nanomoles of PNP generated per microgram of total cellular protein per minute.

Results

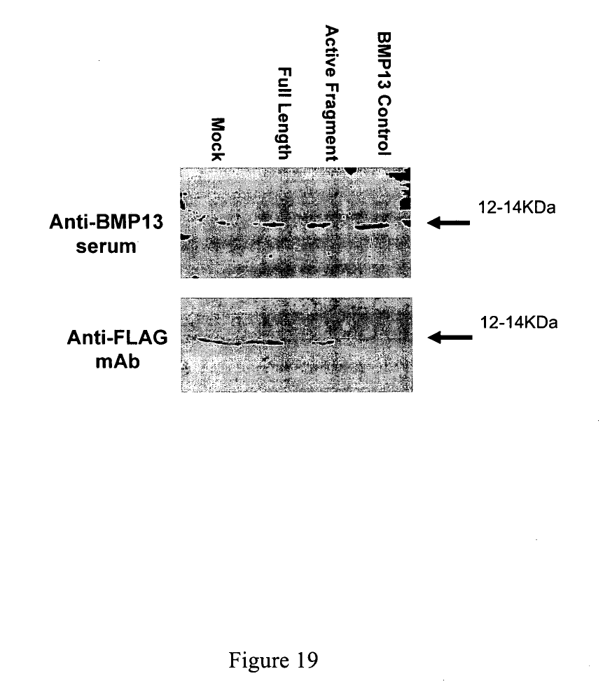

As shown in FIG. 19 Western blotting of supernatant from cells transfected with full-length GDF-6 or active fragment of GDF-6 using anti-GDF-6 antisera detected a band of approximately 12-14 kDa, similar to the positive control. This band was not detected in supernatants from mock transfected cells. The identified GDF-6 bands are also recognised by the FLAG tag-specific mAb (Sigma), however, control protein was not, as expected since the control protein does not comprise a FLAG tag. These data indicate that the GDF-6 protein detected was a product derived from the transfected cDNA.

When protein isolated from CHO cells expression either full-length GDF-6 or active domain of GDF-6 is analyzed using non-reducing PAGE, the size of the protein detected is indicative of a homodimer formation.

In addition the protein purified from the supernatant of transfected cells possessed equivalent/greater alkaline phosphatase activity than commercially obtained protein produced in E. coli (data not shown)

These results indicate that a truncated GDF-6 with a FLAG peptide attached, was produced in CHO cells and secreted into the culture media. The protein forms homodimer reminiscent of normal GDF-6 homodimers, and possessed levels of alkaline phosphatase activity comparable to that of full-length GDF-6.

### EXAMPLE 11

### Treatment of a Sheep Model of IVD Degeneration

Sheep are be purchased from the University farm, Arthursleigh, Australia, and transported to a veterinary centre a minimum of 2 weeks prior to first experimental procedure and housed in a paddock. Each sheep is premedicated with 0.3 mg/kg diazepam and individually taken to the anesthesia induction area immediately prior to the surgical procedure. The jugular vein is catheterized using a 16 G×3.25 cm catheter after local anesthetic is placed under skin using a syringe and 25 gauge needle. Sheep are anaesthetized with 10 mg/kg ketamine given to effect. The sheep are placed in right lateral recumbency and the right caudal quarter of the sheep clipped and aseptically prepared. A straight incision a few fingerbreadths below the costal margin and parallel to lateral border of the erector spinae muscles is made to allow exposure of the lower lumbar vertebrae (L2 to L6). The approach is made retroperitoneally using electrocautery to divide the subcutaneous tissue, fascia, and thoracolumbar aponeurosis and transversalis fascia in line with the skin incision, the peritoneum is protected and reflected anteriorly by blunt dissection. A retractor is placed between rostral and the iliac crest to aid exposure. The vertebral bodies from L3 to L5 are identified and, with a Deaver retractor, the vessels lying anterior to the spine are protected. Once the appropriate involved vertebra is identified, the psoas muscle is elevated bluntly off the lumbar vertebrae and retracted laterally to the level of the transverse process with a Richardson retractor. Bipolar coagulation of vessels around the vertebrae is also performed. The fibrosus annuli of anterolateral discs of L2 to L5 are identified. Annular fibrosus of two non-contiguous lumbar discs per animal. The incision of fibrous annulus is made to a 6 mm depth using a #9 B-P knife blade. The IVD located between the two punctured IVDs is used as a control. A 27 mm×10 mm titanium screw is implanted into the vertebral body at one level for later identification of levels. One of the punctured levels is treated with 300 μg of full-length GDF-6 or an active domain thereof produced essentially as described in Example 10 in saline solution, and the other punctured disc treated with saline control. In both cases the treatment is injected into the nucleus pulposus of punctured discs. After completion of procedure, the wound is closed in layers.

Radiographs (lateral only) are taken prior to waking of animals. Two weeks after surgery and monthly thereafter, radiography are performed on the assigned sheep using 0.3 mg/kg diazepam and 0.2 mg/kg butorphanol intravenously for sedation. The remaining radiographs and CT scans are performed after euthanasia on the assigned days (4, 8 and 18 months). Disc height is also raidographically determined on a monthly basis (lateral view only) and following euthanasia. IVD height is expressed as a disc height index (DHI). The level of degeneration based on the Thompson grade (1=normal, 4=severely degenerated) is also assessed using MRI.

Following four, eight or eighteen months, sheep are euthanized using an overdose of pentobarbitone administered intravenously. After euthanasia the sheep spines are removed for analysis and subjected to CT and MRI scans.

Lumbar vertebral joints are biomechanically tested using an Instron 8874 in 4 modes. Range of motion, constraint to motion, and hysteresis are quantified for the treated joints and compared to controls. Annular tissue samples from treated joint levels and controls are to be isolated and tested in tension to determine ultimate strength and tensile modulus.

Disc tissue collected post-mortem is subject to histological analysis to assess the level of disc degeneration. Spines are removed surgically and muscle tissue removed before being submerged in working formalin solution (10% in 0.1M Phosphate buffer). Spines are labeled according to head/tail orientation. After initial fixation, spines are segmented into individual discs labeled +2, +1, −1, −2 in relation to the position of the titanium screw inserted at surgery, and thus identifiable in respect of the treatment administered. Discs are then de-calcified. Individual discs are sectioned into pieces of tissue no more than 5 mm thick, placed in cassettes for paraffin embedding and thin sectioning. Tissues are then stained with haematoxylin/eosin for tissue architecture analysis.

### EXAMPLE 12

### GDF-6 Regenerates Disc in Asheep Annular Tear Model

12.1 Materials and Methods

Sheep (either n=7; or n=8) underwent left retroperitoneal exposure of their lumbar spine and three discs were exposed. The control disc was either exposed alone i.e., left uninjured and untreated., the other two disces were injured with a No 15 bard Parker Blade to a 6 mm depth and injected with 50-70 μl of saline or injected with 50-70 μl of recombinant human GDF-6 (rhGDF-6). To mark the level of the discs a titanium screw was implanted into a marker vertebral body. Sheep were sacrificed at 3, 4, 6or 8 months.

12.1.1 Method of Evaluation of Early Degeneration

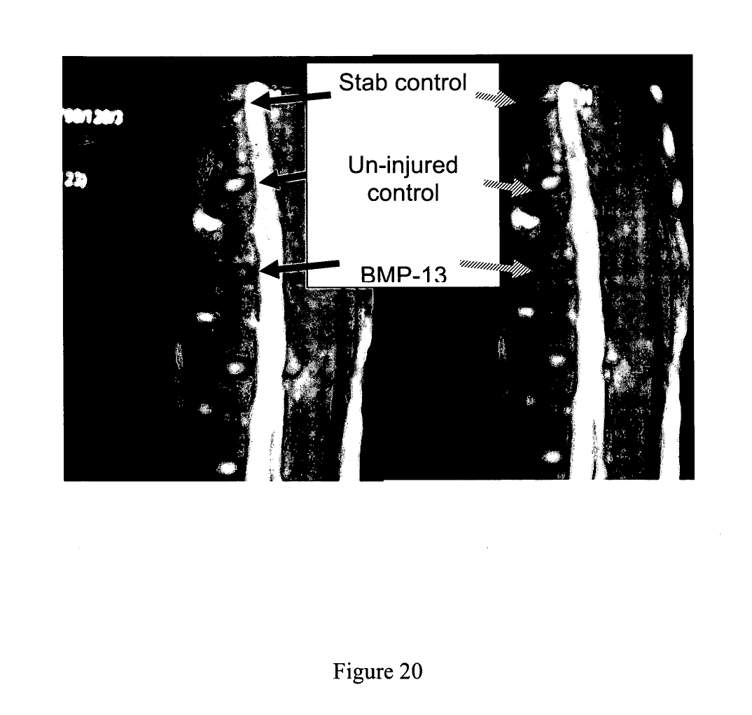

To evaluate early degeneration and its treatment highly sensitive MRI scans were used. Exemplary results are shown in FIG. 20. Degeneration grading was based on visual inspection evaluating predominantly nucleus pulposus hydration, end plate changes and disc height and blinded scoring was performed by two observers. Discs were graded as follows: Good disc (1), somewhat good disc (2), bad disc (3), Very bad disc (4) and finally extremely disc (5).

12.1.2 Method of Histological Evaluation-Haematoxylin and Eosin Staining

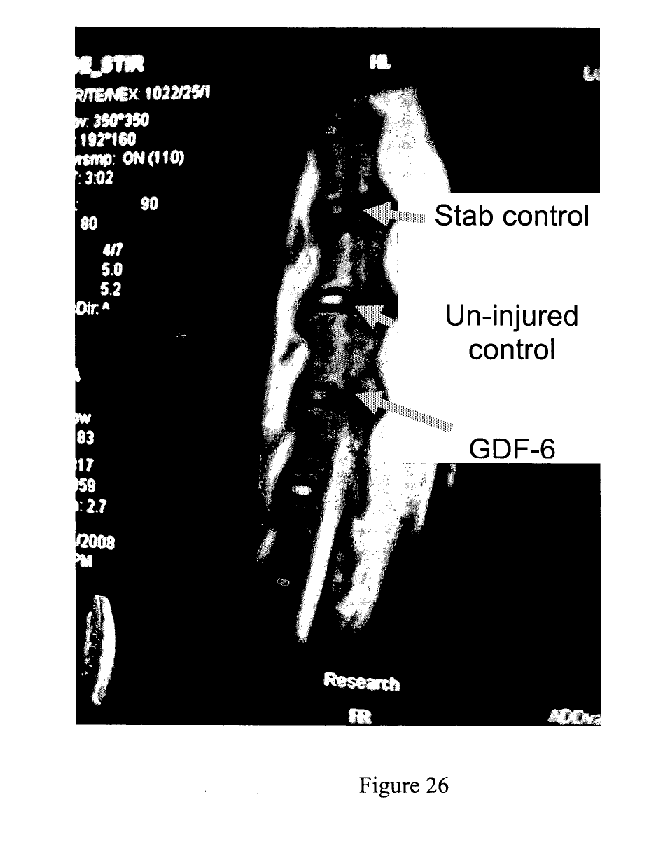

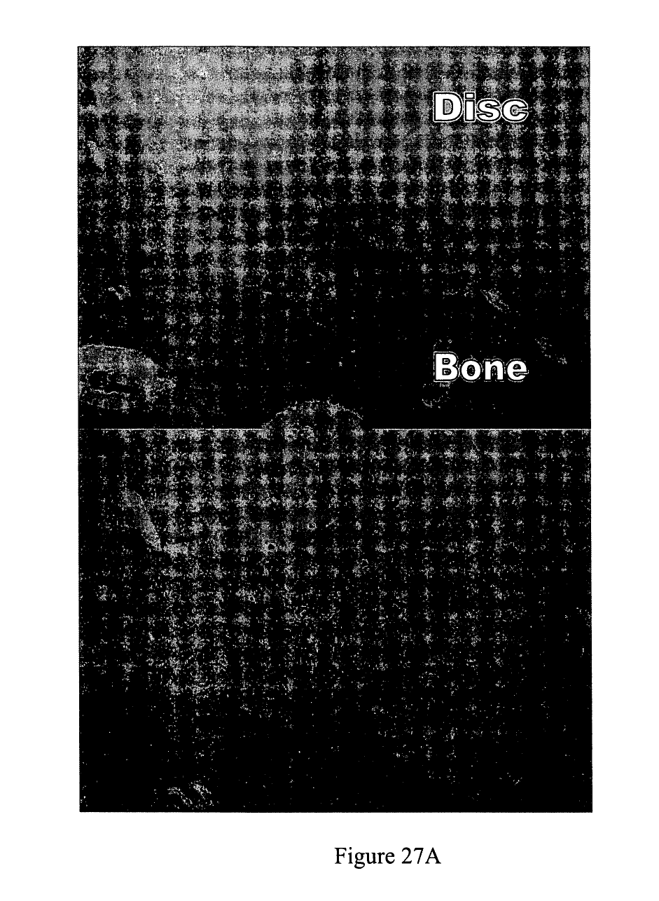

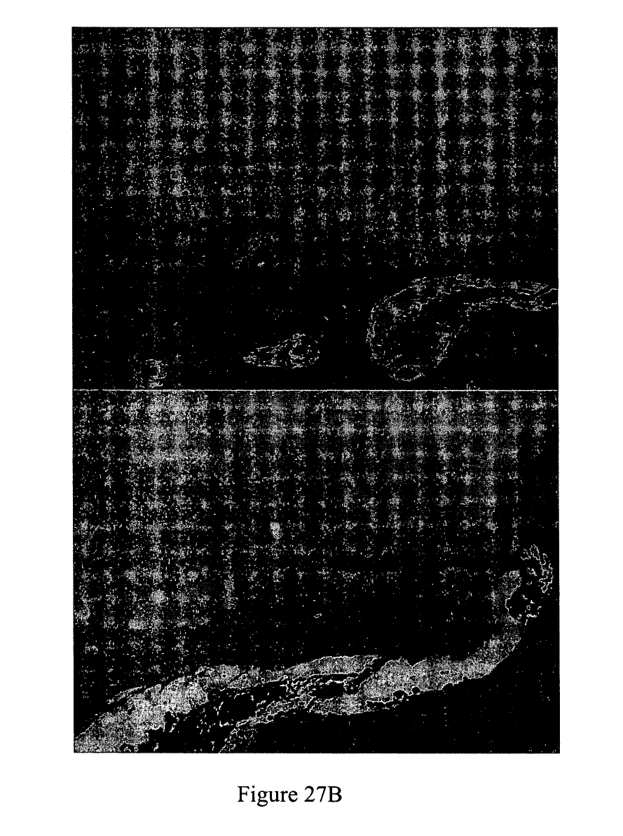

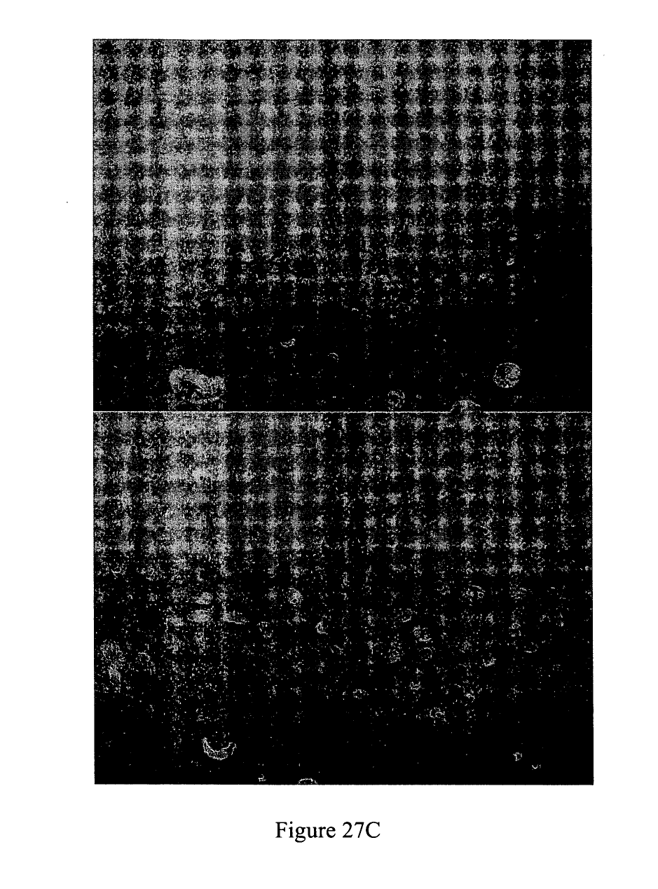

To perform histological evaluations, sheep (n=8) underwent left retroperitoneal exposure of their lumbar spine and three discs were exposed, injured and treated as described above. The sheep were sacrificed at 3 and six months, the spinal column was then cut into motion segments and fixed in formaldehyde subsequently were placed in rapid decalcifying acid solution to soften the bony elements. Sagital and coronal section were made, embedded in paraffin, sectioned on a microtome and sections of 5 um were mounted on slides. Sections were then stained with haematoxylin and eosin. Sections were studied under an Olympus light microspcope at 100× and 400× magnification. Representative MRI scans showing the evaluation of early degenerative changes at the 4 month mark are shown in FIG. 26. Representative images stained with haematoxylin and eosin are shown in FIG. 27A through 27C.

12.1.3 Method of Histological Evaluation-Proteoglycan and Collagen in Annular Tear Model

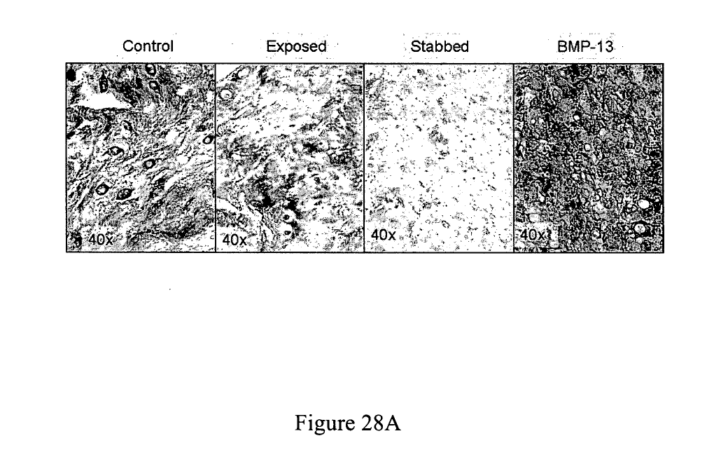

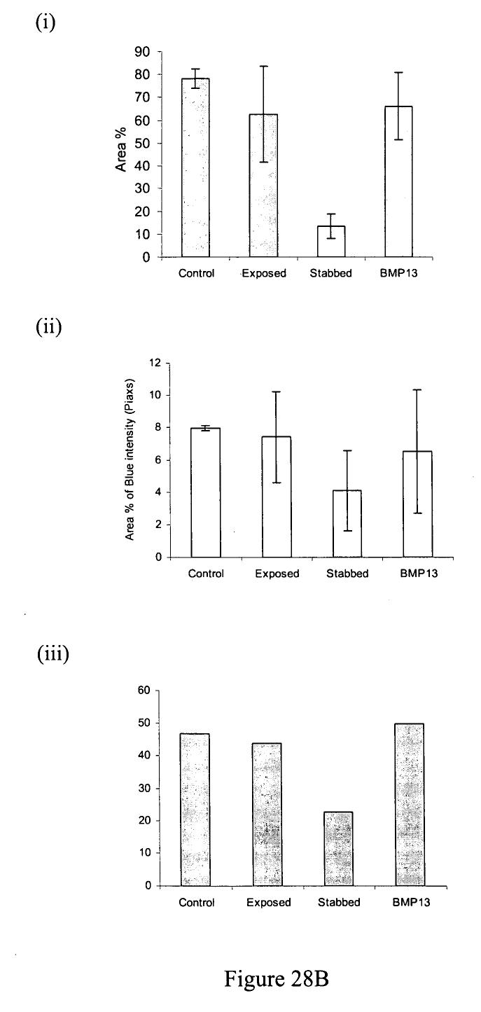

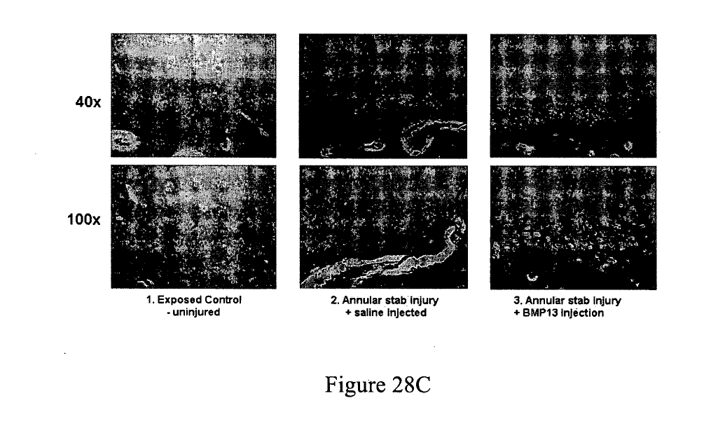

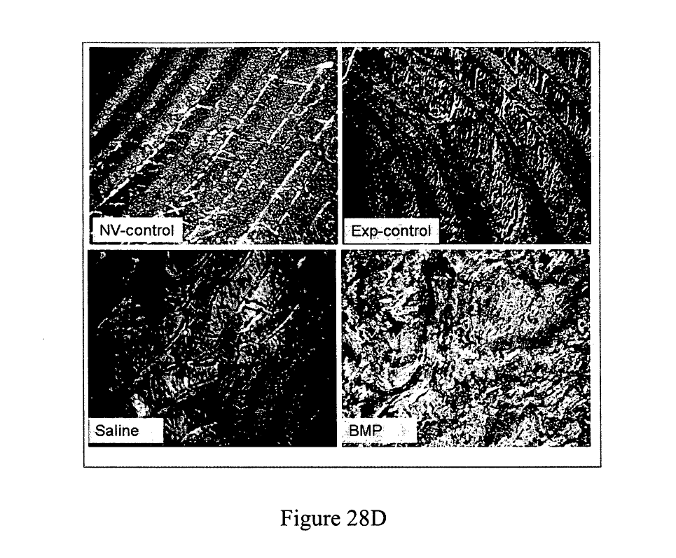

To evaluate the presence of proteoglycan and collagen in the sheep annular tear model, sheep (n=7) underwent left retroperitoneal exposure of their lumbar spine and three discs were exposed, injured and treated as described above. The sheep were sacrificed at 3 and 8 months, the spinal column was removed, fixed in formalin, and the disc tissue was embedded in paraffin, sectioned on a microtome and sections of 5 μm were mounted on slides. Sections were then stained with Alican Blue to detect proteoglycans, stained with Haematoxylin and Eosin as controls to compare tissue architecture and visualized under with polarized light to view collagen. Sections were analysed under an Olympus light microspcope at 40× and 100× magnification. Representative images stained with Alican Blue are shown in FIG. 28A and FIG. 28B. Representative images stained with Haematoxylin and Eosin are shown in FIG. 28C and representative images from viewing under polarized light are shown in FIG. 28D.

12.2 Results

12.2.1 Evaluation of Early Degeneration

At the 4 month mark early degenerative changes were appreciated by both observers. All uninjured controls were Good (1), saline controls were generally graded as very bad (4) and the majority of rhGDF-6 treated discs graded some what good (2). The MRI changes precede the gross degenerative changes (or disc height loss) in early degenerative phases. These results indicate that rhGDF-6 can reduce or delay IVD degeneration and/or enhance IVD regeneration in a sheep annular tear model as seen in the MRI images (FIG. 20 and FIG. 26).

12.2.2 Evaluation of Cellularity by Haematoxylin and Eosin Staining of Discs in the Annular Tear Model

The images in FIG. 27A show the endplate of a disc representing an exposed control without receiving any annular injury (un-injured control). Multiple layers of cells with the cells on the disc side of the endplate appearing to have matured and moved into the disc tissue in an active manner were observed. In particular, resting cells are seen at the base, with larger cells moving towards the centre of the disc. The bony side of the endplate does not show any appreciable vascularisation.

The images in FIG. 27B show the endplate of a disc that received an annular tear with saline injection (stab control). A reduction in the number of cells in the resting basal phase in the end plate region and a decrease in discharge of cells into the discal tissue was observed. There were numerous vascular channels in the bony side of endplate region was also observed, i.e., neovascularization, which is indicative of a rapidly progressive histological degenerative change.

The images in FIG. 27C show the endplate of a disc that are from a disc that received an annular tear and treated with GDF-6. An intense increase in cellularity at the basal layers of the endplate and also in the number of cells moving into the nucleus and annular layers of the disc was observed. This is indicative of an increased proliferative response of the resting or stem cells in the end plate. No vascular response or vascular channel building on the bony side of the end-plate was observed.

These findings demonstrate GDF-6 not only mobilizes and proliferates stem cells (Anabolic) but also suppresses the degeneration and neo-vascularization of end plates (Anti-catabolic) thereby having a two pronged beneficial effect on disc degeneration. Moreover, these findings indicate not only a protective effect of GDF-6 to injury-induced degeneration but also a regenerative response, that in later stages will augment disc height and function by way of restoring viscoelastic properties of the disc.

12.2.3 Histological Evaluation of Proteoglycan and Collagen in Annular Tear Model

At the 3 month mark, an increase in proteoglycan in injured discs which had received injected GDF-6 as compared to injured discs injected with saline, was observed. The presence of proteoglycan was evidenced by a greater intensity of blue staining on the slide, and detected in the Nucleus Pulposus (FIG. 28A and FIG. 28B) and in the Annular Fibrosis. Proteoglycan was markedly reduced in discs following injury and surgical exposure, when compared to untouched controls. In slides stained with haematoxylin and eosin (FIG. 28C), there was an increase in the density of cells originating in the end plate from injured discs that received injections of GDF-6, compared to those which received saline. In addition, slides demonstrated greater vascularisation of the bony-side of the end plate cartilage in injured discs that did not receive GDF-6, when compared to the GDF-6 treated discs. In slides visualized under polarized light (FIG. 28D) an increase in collagen in the annular fibrosis was observed when injured discs were injected with GDF-6. Collagen was reduced in discs following injury, and surgical exposure when compared to untouched controls.

These data indicate that GDF-6 stimulates increased proteoglycan production in the intervertebral disc and increased collagen synthesis in the annular fibrosis of the intervertebral disc. These data further confirm the results supra that GDF-6 stimulates increased numbers of disc cells migrating into the disc nucleus via the end-plate GDF-6 reduced injury-induced end plate vascularisation.

### EXAMPLE 13

### Treatment of Isolated IVD Cells with GDF-6 Induces Extracellular Matrix Production

Harvest and Maintenance of Disc Cell Cultures

Surgically-discarded human disc tissues were collected in sterile saline (0.9% NaCl; Baxter International Inc., Deerfield, Ill., USA) following patient consent. Tissues were repeatedly washed in sterile phosphate buffered saline (Invitrogen) until the solution was clear, then cut into approximately 1 mm² pieces prior to overnight digestion with 0.025% collagenase (Sigma-Aldrich, St Louis, Mo., USA). Following digestion with collagenase, cell suspensions were suspended in 0.02 M HEPES, 2% antibiotic-antimycotic (penicillin/streptomycin/fungizone (P/S/F)) and Hank's balanced salt solution (HBSS) (Invitrogen, Carlsbad, Calif., USA), in a shaking incubator at 37° C. The cells were then harvested with a 1000 rpm centrifugation step for 10 minutes, with the resultant cellular pellet resuspended into fresh culture media (10% fetal calf serum (FCS) (HyClone®, Tauranga, New Zealand) with 1% antibiotic-antimycotic (P/S/F) in DMEM) (Invitrogen) and cultured in monolayers, within a 37° C. cell culture incubator with 5% CO₂.

Trypsinisation and the Passaging of Cells

Cells were grown to confluency and passaged by trypsinisation and re-seeded at a lower density for further culturing or experimentation. Specifically, adherent cultures were washed twice with sterile, pre-warmed phosphate buffered saline for complete removal of existing culture media prior to incubation with trypsinisation solution (0.05% Trypsin-EDTA-4Na, 0.02 M HEPES in HBSS) (Invitrogen), completely covering the cellular surface, in the 37° C. cell culture incubator with 5% CO₂ until the cells were in suspension. Trypsin was then inactivated by addition 10% FCS containing culture media, with the entire contents subjected to a 5 minutes centrifugation step at 1000 rpm for the collection of trypsinised cells. The cells were then resuspended in fresh culture media, with an aliquot removed to determine the viable cellular density by visual-counting of non-trypan blue-stained cells, as described below, and subsequently seeded at an appropriate density into new culture flasks for continued culturing or experimentation.

Non-Trypan Blue Staining for Cell Viability Counting

Equal volumes of cell suspension to 0.4% trypan blue stain (Invitrogen) suspended in HBSS was mixed together and stained for 5 minutes at room temperature prior to applying a small sample under a freshly-placed coverslip on top of a hemacytometer (Bright-line® hemacytometer, Sigma-Aldrich) until the chamber was full. The counting chamber (3×3 mm grid) was visualized microscopically, with the four corner (1 mm) grid counted for the number of viable non-stained cells. The calculation used to determine cell numbers per milliliter and total cells in suspension, was as follows: cells/ml=average count per square (1 mm grid)×dilution factor×10⁴ and total cells=cells/ml×total original volume of cell suspension from which the sample was taken.

GDF-6 Stimulation Studies

Human disc cell cultures from either the annular, nuclear or endplate region were seeded into 60 mm² plates at a density of 1.6×10⁵ cells per plate. On day three post-seeding, cells were stimulated with either media alone or containing 200 ng of GDF-6 (Peprotec Asia, Israel) and left to culture for a period of seven days. Culture media containing the stimulant was changed every three days. Upon the seventh day, the cultures were harvested for either western blotting analysis or real-time RT-PCR analysis.

Western Blot Detection of Matrix Protein Expression

Confluent cells cultured in 75 cm² flasks were washed twice with PBS and 500 μl of homogenization buffer (50 mM Tris pH 7.4, 0.1 mM EDTA, Leupeptin 1 μg/ml, Pepstatin 5 μg/ml, AEBSF 200 μg/ml) (Sigma-Aldrich) was added directly to the cultures and incubated for 20 minutes on ice prior to the removal of cells with a cell scraper. Cellular lysates were then briefly sonicated and stored in −70° C. with individual aliquots for subsequent determination of protein concentration using the Micro BCA™ Protein Assay Kit (Pierce, Rockford, Ill., USA) as well as Western blotting procedures.

For Western blotting, thawed lysates were suspended in an equal volume of 2× sample buffer (4% SDS; 20% glycerol; 25% 0.5 M Tris-HCl pH 6.8; 2-5% of 2-β mercaptoethanol and 0.1% Bromophenol blue)(NuSep). Approximately 12.5 μg of protein extracts were loaded onto and separated by 8% SDS-polyacrylamide gels (LongLife Gels)(NuSep). Proteins were then transferred to PolyScreen® PVDF hybridization membranes (PerkinElmer). Membranes were then incubated in blocking solution (5% skim milk in TTBS) overnight in a 4° C. refrigerator. The TTBS solution consisted of TBS (20 mM Tris, 137 mM NaCl at pH 7.6) with 0.1% Tween20 and was always freshly prepared. For the detection of protein expression, membranes were probed with either goat anti-collagen 2 antibodies (Santa Cruz, Calif., USA), rabbit anti-collagen 1 antibodies (Research Diagnostic INC, NJ, USA), rabbit anti-Sox 9 (antibodies Santa Cruz, Calif., USA) as primary antibody with 15 μl of antibody resuspended in 3 ml of 1% BSA in TTBS for each membrane, placed at room temperature for an hour in a hybridization oven rotating at 7 rpm. The membrane was then washed three times in TTBS for 10 minutes each and labeled with an anti-goat or anti rabbit horseradish peroxidase-conjugated secondary antibody (1.5 μl of antibody in 3 ml of 1% BSA/TTBS) (Chemicon, Temecula, Calif.) for an hour, at room temperature within a hybridization oven rotating at 7 rpm. Three consecutive 15 minute washes in TTBS was then performed, followed by another two 10 minute washes in TBS. The complexes were then detected by the Super Signal Chemilumnescent Substrate system (Pierce) as per manufacturer's instructions.

Following the initial probing of the membrane blot for matrix proteins, antibodies bound to the membrane were stripped to permit subsequent detection of β-actin protein expression, for the normalization of protein loading in each lane. The membrane was submerged in 20 ml of stripping buffer (Pierce) for 20 minutes in a shaking 37° C. incubator at 3.5 rpm. This was followed by a 10 minute wash in TTBS, for a total of three washes, prior to incubation with the mouse monoclonal anti-β-actin primary antibody (Sigma-Aldrich) (1.5 μl of antibody with 10 ml of 1% BSA/TTBS) for 1 hour at room temperature in a hybridization oven, rotating at 7 rpm. The membrane was then washed three times in TTBS, at 10 minutes each and labeled with an anti-mouse horseradish peroxidase-conjugated secondary antibody (1 μl of antibody added to 4.999 ml of 1% BSA/TTBS) (Chemicon, Temecula, Calif.) for 30 minutes at room temperature, within a hybridization oven rotating at 7 rpm. Two consecutive 10 minute washes in TTBS was then performed, followed by another two 10 minute washes in TBS. The complexes were then detected by the Super Signal Chemilumnescent Substrate System (Pierce) as per manufacturer's instructions. Expression levels of collagen-1, collagen-2 or SOX9 were then normalized with respect to β-actin levels to permit comparison of expression levels between samples.

Dose-Response Stimulation of Human IVD Annulus Fibrosis Tissues Invitro

Intervertebral Disc tissue obtained from surgical procedures were separated into Annulus Fibrosus (AF), Nucleus pulposus and End Plate (EP) visually and cultured in vitro following collagenase digestion as described above. Cells were passaged until P2, then stimulated with 200-600ng/mL GDF-6 (E. coli produced, Peprotech) for 7 days. Responses to GDF-6 stimulation were measured by protein expression (western blot) as described above.

Results

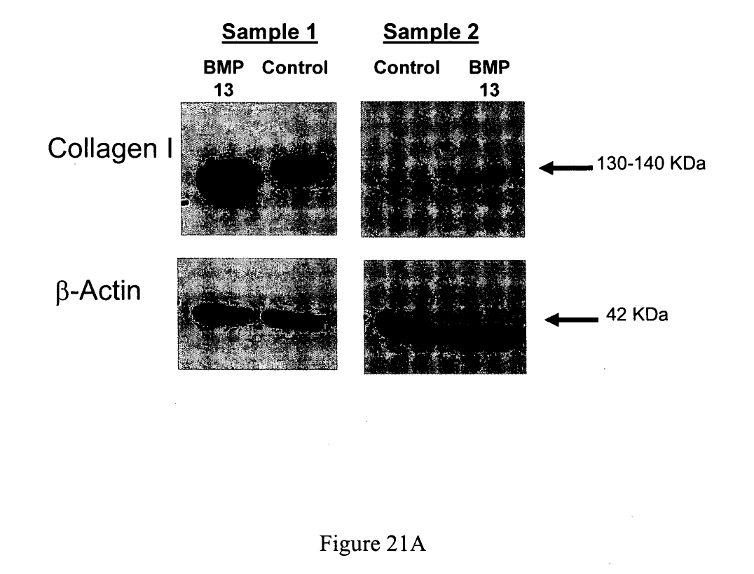

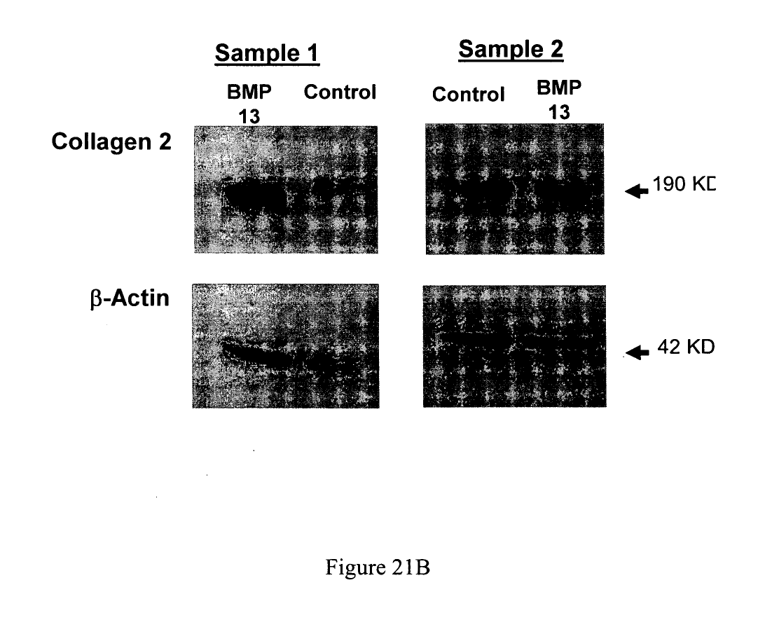

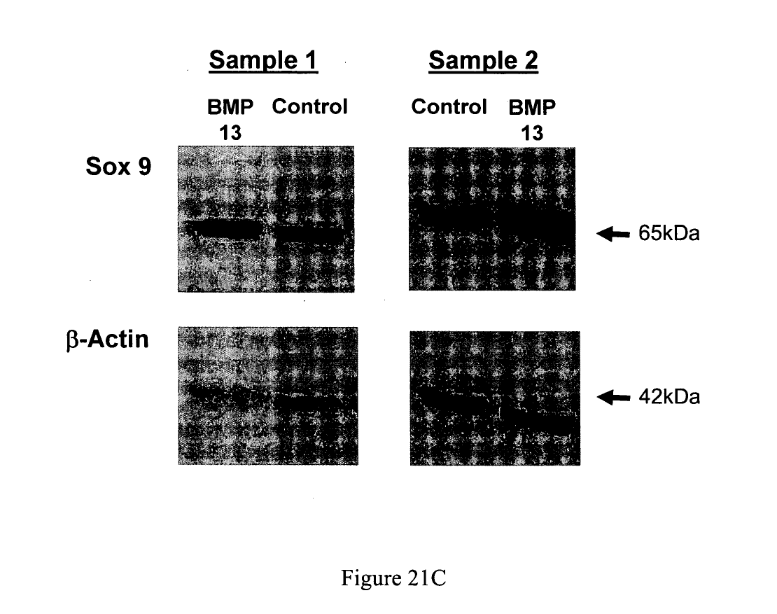

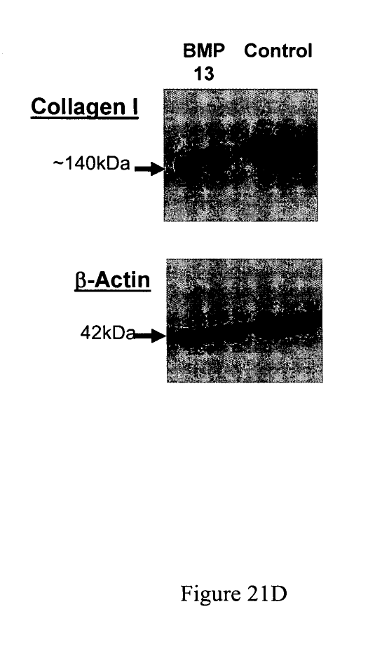

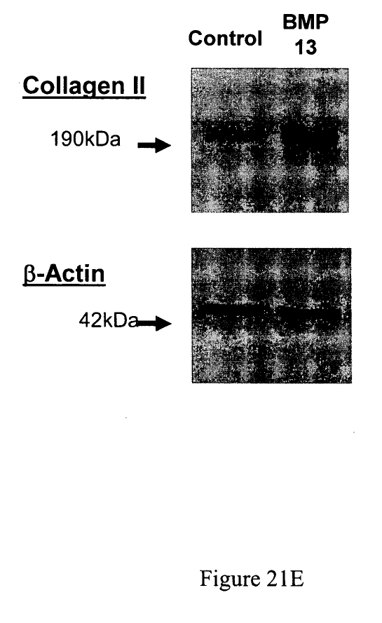

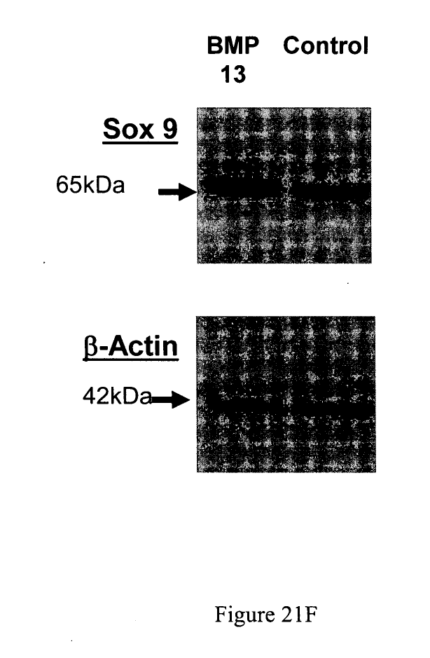

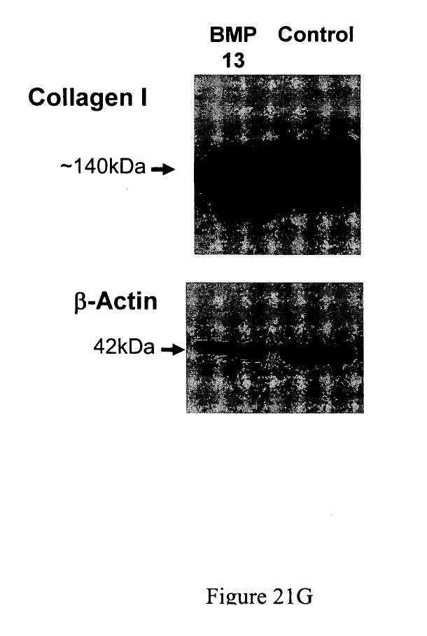

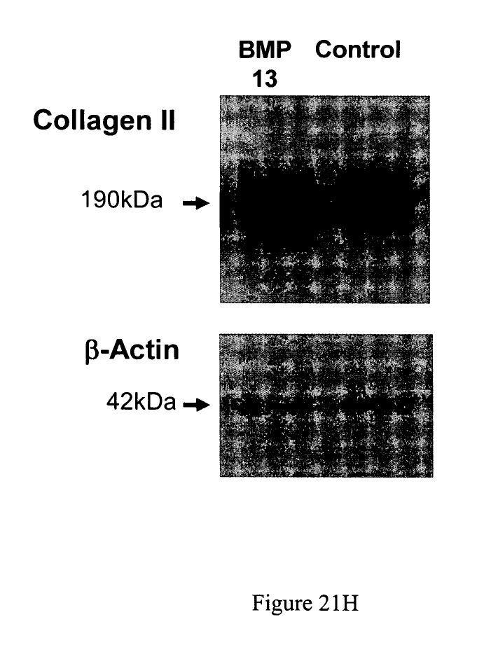

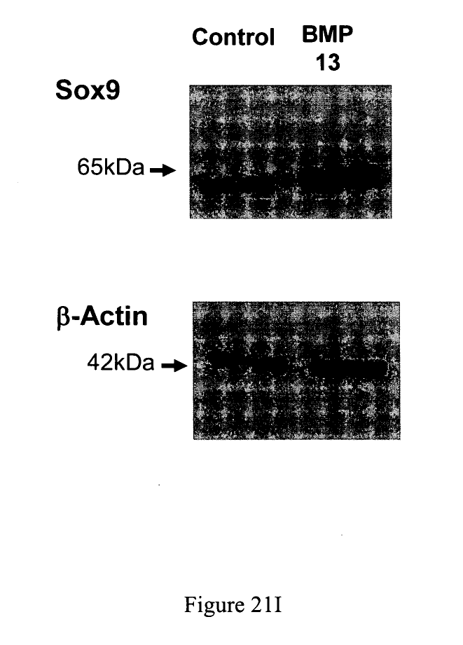

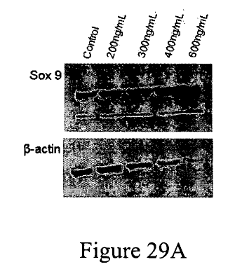

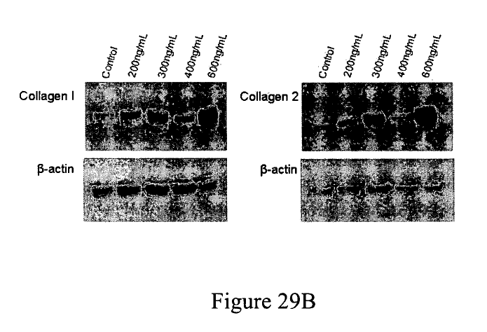

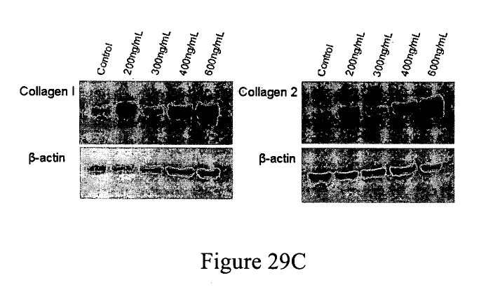

Western blotting of cultured annulus fibrosus cells with GDF-6 demonstrated an increase in the production of collagen I, collagen II and SOX9 proteins by Western blot compared to the level observed in unstimulated cells (as shown in FIGS. 21A-21C). Moreover, western blotting of cultured AF cells stimulated with increasing doses of GDF-6 (FIG. 29A) demonstrated a dose dependent increase in the expression of SOX9 transcription factor protein after 7 days stimulation with GDF-6 in annulus fibrosis cultures.

Accordingly, these results indicate that treatment of primary annulus fibrosus cells with GDF-6 increases expression of extracellular matrix proteins (e.g., collagen-1 and collagen-2) and a transcription factor that enhances expression of proteins involved in extracellular matrix synthesis (SOX9). In this respect, GDF-6 enhances production of the most common form of collagen in the annulus fibrosus, collagen-1.

As shown in FIGS. 21D-21F, GDF-6 enhances the level of expression of collagen-1, collagen-2 and SOX9 in primary nucleus pulposus cells. Accordingly, these results indicate that GDF-6 induces expression of extracellular matrix proteins, e.g., collagen-1 and collagen-2 and proteins involved in enhancing expression of extracellular matrix proteins, e.g., SOX9 in nucleus pulposus cells. As will be apparent from the foregoing description, the extracellular matrix of nucleus pulposus is reduced in a nucleus pulposus of a degenerating or degenerated IVD. Accordingly, these results indicate that GDF-6 is capable of inducing expression of proteins that can slow, reduce or prevent IVD degeneration and/or induce IVD regeneration.

FIGS. 21G-21I show the effect of GDF-6 on expression of collagen-1, collagen-2 or SOX9 in endplate cells (EP). As shown, GDF-6 increases expression of each of these proteins in endplate cells. Moreover, western blotting of cultured EP cells stimulated with increasing doses of GDF-6 in two tissue samples derived from different discs (FIG. 29B-culture 1; and FIG. 29C-culture 2) demonstrated a dose dependent increase in the expression of collagen I and collagen II after 7 days stimulation with GDF-6 in EP cultures.

The results described herein above and shown in FIGS. 21A-21I and 29A-29C demonstrate that GDF-6 increases expression of proteins involved in extracellular matrix production, which are also markers of intervertebral disc cells and chondrocytes.

### EXAMPLE 14

### Analysis of Expression of Extracellular Matrix Markers in Differentiated BM MSC Cells

Cells are produced essentially as described in Example 13, and mRNA expression levels of collagen-1, collagen-2 and Aggrecan assessed as follows:

RNA Extraction from Cultured Cells

At regular times throughout experiments aliquots of cells are pooled together for quantitative real time RT-PCR to detect the level of mRNA's encoding components of extracellular matrix, specifically, aggrecan, collagen type-1 and type-2. RNA extraction from these pooled aliquots is performed with RNeasy Mini Kit (Qiagen, Hilden, Germany), as per manufacturer's instructions. The RNA concentration is then measured with a spectrophotometer at 260 nm with the additional 260 nm/280 nm readings taken for an indication of RNA purity. The isolated RNA preparation is then concentrated in a vacuum pump for 1 hour, reducing the volume to approximately 10 μl for the generation of concentrated RNA stocks.

Generation of cDNA from Isolated RNA (Reverse Transcription)

To completely remove residual DNA from the isolated RNA preparation, 1 μg of RNA is digested with Deoxyribonuclease I, Amplification Grade (Invitrogen) in a 10 μl reaction mixture comprising 1 μl 10× DNase buffer, 1 μl DNase and water for 15 minutes at room temperature. The digestion is inactivated with 1 μl of 25 mM EDTA for 10 minutes at 65° C. Purified RNA preparation was then reverse transcribed (RT) to produce cDNA with the ImProm-II™ Reverse Transcription System (Promega), as per manufacturer's instructions. Briefly, DNase digested RNA mixture (11 μl RNA) is added to 1 μl of Oligo(dT)₁₅ primers and incubated for 70° C. for 5 minutes, with a further 5 minutes incubation on ice. The mixture is kept on ice with a brief spin down to collect any condensation prior to adding the freshly made reverse transcriptase master mix consisting of 3.8 μl MgCl₂, 1 μl dNTP, 1 μl RT and 4 μl 5× buffer. The reaction mixture is then placed in a standard PCR machine for the generation of cDNA with the following program: 25° C. for 5 minutes, 42° C. for 60 minutes, 70° C. for 15 minutes; with the subsequent tubes of cDNA stored in a 4° C. refrigerator until use.

Real-Time SYBER Green Polymerase Chain Reaction

For real-time polymerase chain reaction (PCR), each reaction mix consists of 1 μl forward primer, 1 μl reverse primer, 12.5 μl Platinum® Syber® Green qPCR SuperMix UDG (Invitrogen) and 6.5 μl water made up into a master mix for the total number of reactions performed. The cDNA stock is diluted 1:2 for use in real time PCR reactions, with 4 μl of cDNA (1:2) added to 21 μl of master mix for each reaction, whereby triplicate reactions are set up for every sample by the CAS-1200 robotic liquid handling system (Corbett Robotics, Queensland, Australia). The sequences of the primers used for each gene of interest are as follows: collagen-type 1 forward primer AGACATCCCACCAATCACCT (SEQ ID NO: 26) and reverse primer AGATCACGTCATCGCACAAC (SEQ ID NO: 27); collagen-type 2 forward primer GTGACAAAGGAGAGGCTGGA (SEQ ID NO: 28) and reverse primer ACCTCTAGGGCCAGAAGGAC (SEQ ID NO: 29); aggrecan forward primer TCAACAACAATGCCCAAGAC (SEQ ID NO: 30) and reverse primer AAAGTTGTCAGGCTGGTTGG (SEQ ID NO: 31); house keeping gene, GAPDH forward primer AATCCCATCACCATCTTCCA (SEQ ID NO; 32) and reverse primer TGGACTCCACGACGTACTCA (SEQ ID NO: 33). Primer stocks are all adjusted to 50 μM concentrations, for use in the real time PCR reactions as described above. The completed reaction mixtures are then placed in a Rotor-Gene Thermal cycler (Corbett Research, Sydney, Australia) and a touchdown-PCR program is performed comprising two initial hold steps at 50° C. and 95° C. held at 2 minutes each, followed by 40 cycles of the PCR program: denaturation at 95° C. for 15 seconds, annealing and elongation temperature of 60° C. for 30 seconds. The resultant data generated are visualized, and a threshold at the exponential phase of amplification was set for the collection of cycle times of each gene in every sample tested for subsequent quantitative analysis of gene expression.

Relative Quantification of Gene Expression

Gene expression of protein stimulated-cells relative to the unstimulated cells is analyzed using the relative expression software tool (REST®) Statistical significance is determined by the pair wise fixed reallocation randomization test provided with the software.

### EXAMPLE 15

### The Role of GDF-6 in Chondrogenic Differentiation of Bone Marrow Mesenchymal Stem Cells (BM MSCs)

Differentiation of BM MSCs

BM MSCs at Passage 3-4 were trypsinized using standard method for differentiation assays. For chondrogenic differentiation, MSCs at 1×106 cells/tube were centrifuged to form pellet or suspended in a solution of 1.2% (w/v) low viscosity sodium alginate in 150 mM NaCl, at the density of 5×10⁶/ml. Alginate beads were formed by pressing the cell suspension dropwise into 102 mM CaCl₂ solution through a syringe with a needle. The beads formed instantly and were placed in 12-well plates after washing with 150 mM NaCl solution. Embedded MSCs were differentiated using standard induction medium containing 10 ng/ml of recombinant human TGF-β3 or 300 ng/ml of GDF-6 individually or in combination (TGF-β3&GDF-6). For cell recovery, the cell beads were washed twice in PBS and incubated in 55 mM of Na-citrate solution, pH 7.4 at 37° C. until beads were solubilized and the alginate was removed by centrifugation. Undifferentiated MSCs were cultured in parallel in growth medium as negative control. Cells were kept at 37° C., 5% CO2 for up to 21 days and the media were changed twice weekly.

RNA Extraction from Cultured Cells

At regular times throughout experiments aliquots of cells were pooled together for quantitative real time RT-PCR to detect the level of mRNA's encoding components of extracellular matrix, specifically, aggrecan, collagen type-1 and type-2. RNA extraction from these pooled aliquots was performed with RNeasy Mini Kit (Qiagen, Hilden, Germany), as per manufacturer's instructions. The RNA concentration was then measured with a spectrophotometer at 260 nm with the additional 260 nm/280 nm readings taken for an indication of RNA purity. The isolated RNA preparation was then concentrated in a vacuum pump for 1 hour, reducing the volume to approximately 10 μl for the generation of concentrated RNA stocks.

Generation of cDNA from Isolated RNA (Reverse Transcription)

To completely remove residual DNA from the isolated RNA preparation, 1 μg of RNA was digested with Deoxyribonuclease I, Amplification Grade (Invitrogen) in a 10 μl reaction mixture consisting of 1 μl 10× DNase buffer, 1 μl DNase and water for 15 minutes at room temperature. The digestion was inactivated with 1 μl of 25 mM EDTA for 10 minutes at 65° C. Purified RNA preparation was then reverse transcribed (RT) to produce cDNA with the ImProm-II™ Reverse Transcription System (Promega), as per manufacturer's instructions. Briefly, DNase digested RNA mixture (11 μl RNA) was added to 1 μl of Oligo(dT)₁₅ primers and incubated for 70° C. for 5 minutes, with a further 5 minutes incubation on ice. The mixture was kept on ice with a brief spin down to collect any condensation prior to adding the freshly made reverse transcriptase master mix consisting of 3.8 μl MgCl₂, 1 μl dNTP, 1 μl RT and 4 μl 5× buffer. The reaction mixture was then placed in a standard PCR machine for the generation of cDNA with the following program: 25° C. for 5 minutes, 42° C. for 60 minutes, 70° C. for 15 minutes; with the subsequent tubes of cDNA stored in a 4° C. refrigerator until use.

Real-Time SYBER Green Polymerase Chain Reaction

For real-time polymerase chain reaction (PCR), each reaction mix consisted of 1 μl forward primer, 1 μl reverse primer, 12.5 μl Platinum® Syber® Green qPCR SuperMix UDG (Invitrogen) and 6.5 μl water made up into a master mix for the total number of reactions performed. The cDNA stock was diluted 1:2 for use in real time PCR reactions, with 4 μl of cDNA (1:2) added to 21 μl of master mix for each reaction, whereby triplicate reactions were set up for every sample by the CAS-1200 robotic liquid handling system (Corbett Robotics, Queensland, Australia). Primers were used to amplify cDNA produced from transcripts of markers of chondrogenic cells (collagen II, aggrecan and Sox9). Primer stocks were all adjusted to 50 μM concentrations, for use in the real time PCR reactions as described above. The completed reaction mixtures were then placed in a Rotor-Gene Thermal cycler (Corbett Research, Sydney, Australia) and a touchdown-PCR program was performed consisting of two initial hold steps at 50° C. and 95° C. held at 2 minutes each, followed by 40 cycles of the PCR program: denaturation at 95° C. for 15 seconds, annealing and elongation temperature of 60° C. for 30 seconds. The resultant data generated were visualized, and a threshold at the exponential phase of amplification was set for the collection of cycle times of each gene in every sample tested for subsequent quantitative analysis of gene expression.

Relative Quantification of Gene Expression

Gene expression of protein stimulated-cells relative to the unstimulated cells was analyzed using the relative expression software tool (REST®) Statistical significance was determined by the pair wise fixed reallocation randomization test provided with the software.

Results

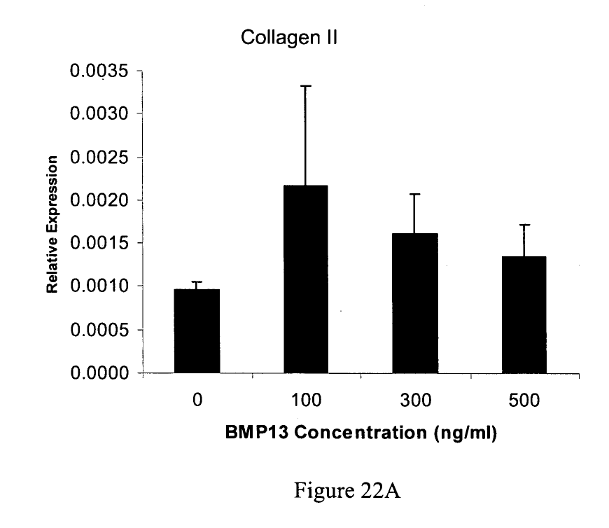

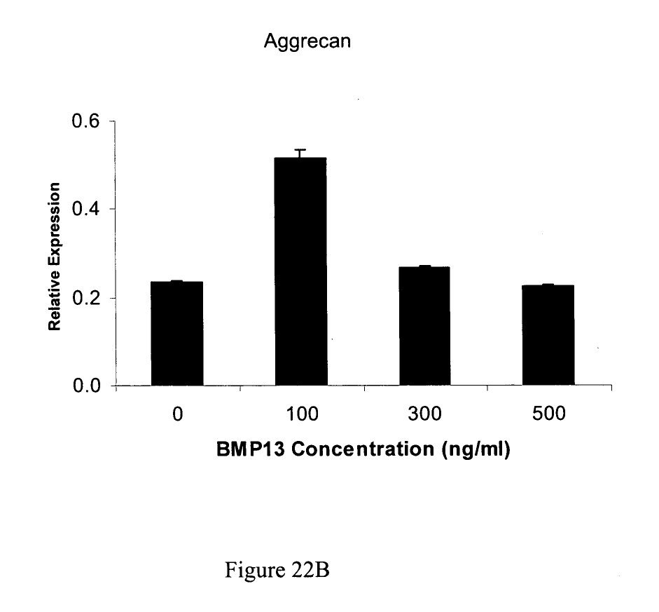

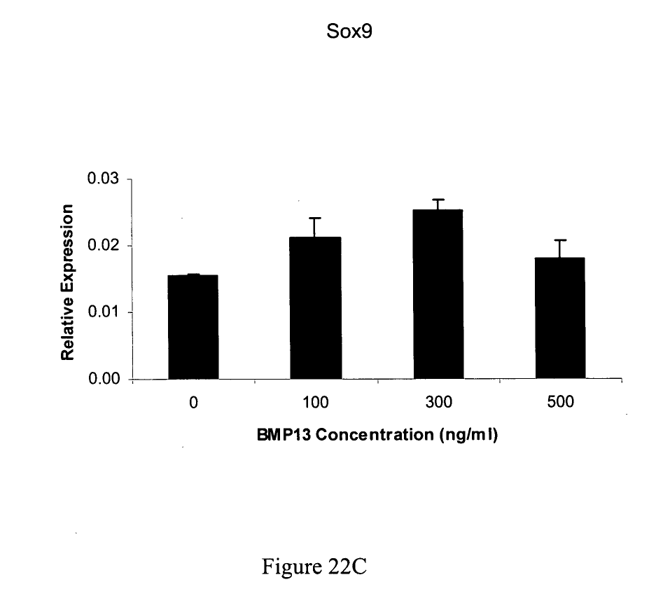

As shown in FIGS. 22A-22C GDF-6 induces expression of chondrogenic genes collagen II, Aggrecan and Sox9. These data indicate that GDF-6 is capable of inducing BM MSCs to differentiate into chondrogenic-like cells. For example, a concentration of 100 ng/ml of GDF-6 (GDF-6)increases expression of Aggrecan, and a concentration of at least 100 ng/ml increases expression of Sox9.

### EXAMPLE 16

### Effect of GDF-6 on Migration and Growth of Human Bone Marrow Mesenchymal Stem Cells (BM-MSC) In Vitro

Gene Expression Analysis

BM MSC were cultured in expansion media and harvested at passage 2 and mRNA prepared for analysis of gene expression levels using real-time PCR. BMP2, 7 and 13 were detected at day 1, 3, 5, and 7 of culture and expressed relative to the house-keeping genes GAPDH and HPRT.

Cell Migration Assays

Cultured human BM-MSC were harvested from flasks by trypsin digestion and resuspended in DMEM/0.1% FBS before seeding (2×104 cells/well) in collagen IV-(Sigma) coated transwells (Costor 3422). Cells were allowed to settle (incubation 30 minutes) then 600 μl of media containing 0, 100, 300 or 500 ng/mL rhGDF-6 (Peprotech) was added to the lower chambers of the transwells. Following overnight (12-16 h) incubation, transwell membranes were washed, cells were fixed and stained, and the total number of migrated cells (on the bottom face of the membrane) was determined.

Cell Growth Assays

Human BM MSCs were seeded in culture flasks (duplicates) at 2000 cells/cm2 and treated with 0, 100, 300 and 500 ng/ml of recombinant human GDF-6 for 3 or 6 days. Following treatment, cells were trypsinized, collected at each time point and counted by trypan blue exclusion method under a hemocytometer. The total cell number was used for comparison between GDF-6 treated and untreated BM MSCs.

Results

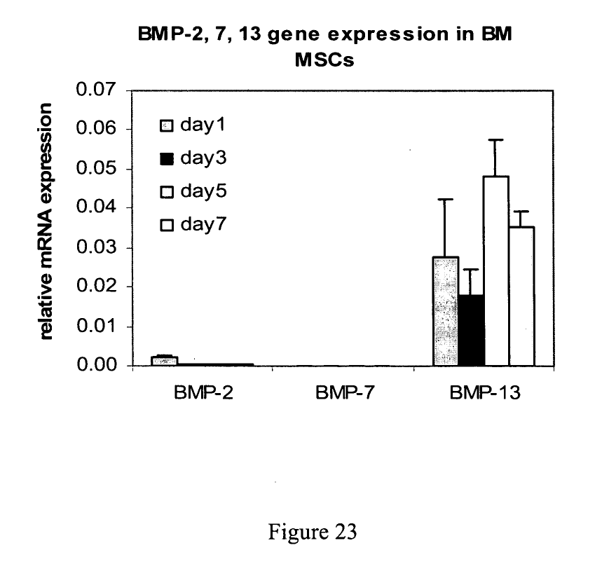

Expression of BMPs 2 and 7 and GDF-6 genes were detected in cultured human BM MSC at day 1, 3, 5 and 7 (as shown in FIG. 23). At all time points GDF-6 was expressed at higher levels than the other BMPs, peaking at day 5.

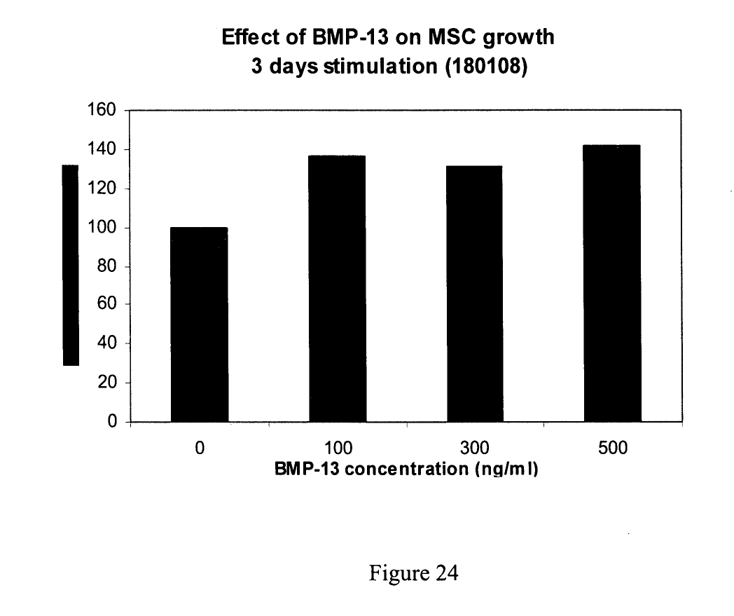

Culture of MSC in the presence of human GDF-6 at all concentrations tested resulted in greater cell numbers than media alone (FIG. 24). Cell numbers did not appear to be greater with increasing dose in the range 100-500 ng/mL. Thus GDF-6 can stimulate increased growth of mesenchymal progenitor cells.

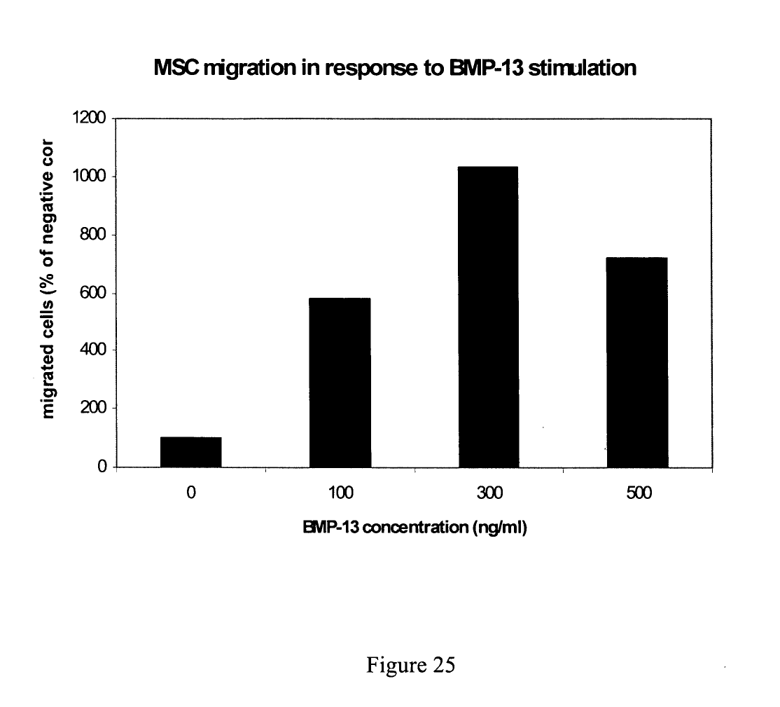

The presence of GDF-6 in the lower chamber of BM MSC transwell cultures induced the migration of MSC towards the source of the GDF-6 (FIG. 25). This indicates that GDF-6 acts as a chemoattractive agent for BM-MSC cells. The number of cells migrating towards the GDF-6 appear to follow a dose response, with 300 ng/mL inducing maximum migration.

These results indicate that GDF-6 is expressed in BM MSC cultures at a higher level than BMP2 or BMP7, perhaps indicating increased importance in progenitor cell function. GDF-6 also appears to stimulate cell growth in BM MSC cultures at 100 ng/mL and to act as a chemottractive agent for BM-MSC cells. Chemoattraction was dose dependent, peaking at 300 ng/mL.

### EXAMPLE 17

### Expression of MSX1 and/or MSX2 in IVD Cells

Adenovirus carrying expression constructs encoding MSX1 or MSX2 Adenovirus carrying a cDNA encoding MSC1 or MSX 2 are produced by Applied Biological Materials (ABM) Inc.

Isolation and Transduction of IVD Cells

Surgically discarded human disc tissues are categorized by the grade of degeneration as well as the age of the patent. Cells are visually separated into annulus fibrosus or nucleus puplosus cells.

Harvested cells are maintained and transduced with adenovirus carrying the expression construct encoding MSX1 and/or MSX2 in both monolayer and 3D-alginate cultures to obtain more comprehensive data regarding cells under active proliferation (monolayer) and in more physiological settings (3D-alginate).

Post-transfection, cells are harvested at 24, 48 and 72 hours, 1, 2 and 4 week time points to enable the analysis of the beneficial effects MSX 1 or 2 has on discal cells in vitro using the assays described below.

Cell Proliferation Studies

To determine the effect of MSX 1/2 on cellular proliferative capacity, periodic cell counts are performed at each cellular passage between transfected and non-transfected controls. A graphical representation of cell numbers depicts an accumulated growth curve over time for each of the groups. Additionally, DNA synthesis is assessed as a measure of mitotic activity using Cell Proliferation ELISA, BrdU (Chemiluminescence) kit, Roche Applied Science (Australia).

Cell Viability Studies

To ensure Ad-MSX 1/2 has no cellular toxicity effects in vitro viability assays are performed with Celltiter 96 Aqueous One Solution, Cell Proliferation assay, Promega.

Secondly, anti-apoptotic ability of Ad-MSX-1/2 is tested by TNF-α- or IL-1-induced apoptosis of cells prior to Ad-MSX 1/2 transfection with the subsequent measurements of cellular viability (as described above) and apoptosis (In Situ Cell Death Detection Kit, Apoptotic DNA Ladder Kit and Annexin-V-FLUOS Staining Kit, Roche).

In vivo discal cells are often in hypoxic environments whereby low oxygen content is common even in normal discs. To detect the extent of protection by Ad-MSX-1/2 under hypoxic conditions transfected cells are incubated in 2-5% O₂ levels, which is representative of physiological oxygen levels in normal to degenerated discs. The level of cellular apoptosis is then determined as described supra. The level of extracellular matrix synthesis is also determined as described infra.

Cell Synthetic Activities

The effects of Ad-MSX-1/2 on cellular synthetic activity is detected at both the mRNA and protein levels. TaqMan™ real-time RT-PCR is performed with primers and probes for Aggrecan, Collagen 1 and Collagen 2 specifically designed by Applied Biosystems. The protein levels are detected by aggrecan, collagen-1 and collagen-2 antibody detection through flow cytometry and immunohistochemistry.

### EXAMPLE 18

### Differentiation of BM MSCs into Nucleus Pulposus-Like Cells Using GDF-6

Tissue Samples

Human bone marrow was collected from iliac crest of 6 haematologically normal donors. Human IVD tissue was collected from 8 patients undergoing lumbar disc replacement. The nucleus pulposus tissue was immediately separated from annulus fibrosus after surgery. Half of the nucleus pulposus tissue was used for RNA extraction and the other half for nucleus pulposus cell isolation.

Cell Isolation and Cultivation

BM MSCs were isolated by immunodepletion, Ficoll-Paque density gradient centrifugation and plastic adhesion essentially as described in Tao et al., Dev. Growth Differ., 47: 423-433, 2005. Briefly, fresh bone marrow specimens were incubated for 20 min with an antibody cocktail available from StemCell Technologies (Vancouver, Canada) to remove mature lineage-committed cells. Ficoll-Paque (GE Healthcare, Uppsala, Sweden) density gradient centrifugation was then performed to separate mononuclear cells from antibody cross-linked cells and enriched cells from the interface were seeded in plastic culture ware. The cells were cultured in growth medium (essentially as described in Tao et al., supra) comprising of about 51% Dulbecco's Modified Eagle's Medium-low glucose (DMEM-LG), 10% fetal bovine serum (FBS; Invitrogen, Carlsbad, Calif., USA), about 34% MCDB-201 medium, 1% insulin transferrin selenium (ITS), 1% linoleic acid/bovine serum albumin (BSA), 1 nM dexamethasone, 32 μg/ml ascorbic acid 2-phosphate (Sigma-Aldrich, St. Louis, Mo., USA) and incubated at 37° C. with 5% CO₂. After about 3 days, non-adherent cells were discarded and adherent BM MSCs were cultured to about 80% confluence with medium changed twice weekly.

Nucleus pulposus cells were isolated by overnight digestion with 0.025% collagenase solution and collected by centrifugation. Nucleus pulposus cells were cultured in DMEM-LG medium containing about 32 μg/ml ascorbic acid 2-phosphate and 10% FBS. Passage 0 cells were used as positive control cells in the experiments described below.

Flow Cytometry Analysis

MSCs are trypsinized and washed with PBS containing 10% FBS and incubated with human AB plasma at 4° C. for 30 min. After washing with FACS buffer (PBS containing 13.6 mM Tri-sodium citrate and 1% BSA), MSCs (1×105 per tube) are resuspended in 50 μl FACS buffer and labeled with 5 μl of fluorescein isothiocyanate (FITC), phycoerythrin (PE) or peridinin chlorophyll protein (PerCP) conjugated monoclonal antibodies in dark at 4° C. for 30 min. Antibodies used include anti-CD29, anti-CD73, anti-CD45, anti-CD14, anti-CD34, anti-CD166, anti-HLA Class I, anti-HLA Class II (BD Biosciences Pharmingen, San Jose, Calif., USA), anti-CD44 and anti-CD105 (Chemicon, Temecula, Calif., USA). The cells are analyzed on a FACSCalibur flow cytometer (BD Biosciences).

Chondrogenic Differentiation in Alginate Bead 3D Culture

MSCs expanded in vitro were encapsulated in alginate beads. Briefly, cells were trypsinized and suspended in a solution of about 1.2% (w/v) low viscosity sodium alginate in 150 mM NaCl, pH 7.4, at the density of 5×10⁶/ml for differentiation and 1×10⁶/ml for undifferentiated control. Alginate beads were produced by gently pressing the cell suspension dropwise into 102 mM CaCl₂ solution through a syringe with a 19 G needle. The hydrogel beads formed instantly and were placed in 12-well plates after washing 3 times with 150 mM NaCl solution.

NP chondrogenic differentiation was performed using medium induced by adding serum-free media containing DMEM-high glucose supplemented with 100 nM dexamethasone, 50 μg/ml ascorbate 2-phosphate, 40 μg/ml L-proline, 1.25 mg/ml BSA, 5.35 μg/ml linoleic acid, 1% ITS solution and recombinant GDF-6 or an active fragment thereof produced as described in Example 10 optionally combined with recombinant human (rh) TGF-β3 or combined with rhTGF-β3 and rhBMP-2 (TGF-62 3&BMP-2, R&D Systems, MN, USA), or cells were incubated with rhTGF-β3 and rhBMP-2 in the absence of GDF-6 or the active fragment. Undifferentiated MSCs were cultured in parallel in growth medium. Cells were kept at 37° C., 5% CO2 for up to 21 days. The media was changed twice weekly. For cell recovery, the cell beads were washed twice in PBS and incubated in 55 mM of Na-citrate solution, pH 7.4 at 37° C. for 10 min. The solubilized alginate was removed by centrifugation and the cell pellet was washed with PBS.

RNA Extraction, cDNA Synthesis and Real-Time PCR

Total RNA was isolated from MSCs, nucleus pulposus tissue and cultured nucleus pulposus cells using TRIzol reagent (Invitrogen) and RNeasy kit (Qiagen, Dusseldorf; Germany) essentially according to manufacturers' instructions. Copy DNA (cDNA) is prepared using SuperScript III first-strand synthesis system (Invitrogen) essentially according to manufacturer's instructions. Briefly, total RNA (1 μg) is reverse transcribed in a final volume of 20 μl using M-MLV reverse transcriptase (200 units) and a mixture of random hexamers (50 ng) and Oligo(dT)20 (50 pmol) as primers. Samples are incubated at 25° C. for 10 min, 50° C. for 50 min and then heated to 85° C. for 5 min. A dilution of the resulting cDNA is used in 20 μl-reactions for real-time PCR analysis in a Rotor-Gene RG3000 system (Corbett Life Science, Sydney). Primers to amplify transcripts from genes encoding collagen-2, aggrecan and Sox-9, which are markers of chondrocytic cells are designed using published mRNA sequences. To exclude possible genomic DNA contamination, the RNA is treated with DNase and primers are designed to be intron-spanning. The thermal profile for all reactions was as follows: 5 min at 95° C., followed by 40 amplification cycles of 15 sec at 95° C., 30 sec at 60° C. and 30 sec at 72° C. Relative expression levels are calculated as a ratio to the average value of house-keeping genes, glyceraldehyde-3-phosphate dehydrogenase (GAPDH) and hypoxanthine phosphoribosyltransferase 1 (HPRT1).

Histology and Immunohistochemistry

Alginate beads encapsulated with MSCs were fixed in 10% neutral buffered formalin for 1 h and embedded in paraffin. Sections of 4 μm thickness were cut and mounted on Super Plus slides (Lomb Scientific, Australia). Sections were dewaxed in xylene and hydrated with graded ethanol before staining. Hematoxylin-eosin (H&E) staining was carried out for general histological examinations. For Alcian blue staining, slides were stained in 1% Alcian blue solution for 15 min and nuclei are counterstained with 0.1% nuclear fast-red solution. For immunohistochemical staining, slides are equilibrated in Tris-HCl (pH 7.6) buffer. Endogenous peroxidases are scavenged with 3% (v/v) H₂O₂ and non-specific binding is blocked by incubation in 10% skim milk in Tris-HCl buffer. Sections are incubated with primary goat anti-human type II collagen polyclonal antibodies or goat anti-human SOX9 polyclonal antibodies (Santa Cruz Biotechnology, CA, USA) or mouse-anti-human collagen-2 monoclonal antibody for 1 h at room temperature. Slides are treated with MULTILINK solution (DAKO, Australia) followed by streptavidin-conjugated peroxidase incubation. The sections are visualized with 3,3′-diaminobenzidine hydrochloride solution and counterstained with Haematoxylin. The primary antibody is omitted for the negative controls.

Western Blot Analysis

Cells are rinsed with cold PBS and lysed in CelLytic-M solution containing protease inhibitors (Sigma-Aldrich). Equal amount of proteins are electrophoresed on 8-12% gradient SDS-polyacrylamide gels (Invitrogen). Proteins are transferred by electroblotting to PVDF membranes, which are then blocked with 5% skim milk in Tris-HCl buffered saline (TBS; 20 mM Tris, pH 7.6, 0.15 M NaCl) overnight at 4° C. Membranes are incubated with goat anti-human type II collagen or rabbit anti-human SOX9 polyclonal antibodies (Santa Cruz Biotechnology) in TBS buffer containing 0.1% Tween-20 (TTBS) for 2 h at room temperature. Alpha-tubulin or β-actin is detected as reference protein. After washing and incubation with fluorescent dye-conjugated secondary antibodies, immunolabeling is detected using the Odyssey infrared imaging system (LI COR Biosciences, Nebraska, USA). Alternatively, bound antibodies are labeled with an anti-goat horseradish peroxidase-conjugated secondary antibody. Following washing the resulting antibody complexes are detected using the Super Signal Chemilumnescent Substrate System (Pierce) as per manufacturer's instructions.

35S-Sulfate Incorporation

The cell function of differentiated MSCs is investigated in vitro by detecting the biosynthesis of proteoglycans using 35S-sulfate incorporation assay essentially as described in Collier et al., Ann. Rheum. Dis., 48: 37-381, 1989 . Briefly, the alginate beads containing differentiated MSCs are incubated with 20 μCi/well of ³⁵S-sulfate (GE Healthcare) at 37° C. for 24 h. Following release from alginate beads, the cells are harvested and resuspended in papain digestion buffer containing 2 μl of papain suspension per 1 ml of PBS, pH 6.2, 5 mM L-cysteine and 10 mM EDTA at 60° C. for 3 h to release glycosaminoglycans. An aliquot is separated for DNA determination. Newly synthesized ³⁵sulfated glycosaminoglycans are separated from free ³⁵SO₄ by a precipitation procedure. Samples are then counted in an automated Scintillation Analyzer and normalized by DNA concentration. The fold change of relative counts represents the change in proteoglycan synthesis.

Results

Results showing that cells incubated in the presence of GDF-6 or an active fragment thereof express increased levels of collagen-2 and aggrecan and Sox9 compared to undifferentiated MSCs indicate that GDF-6 or the active fragment thereof induce MSCs to differentiate into a nucleus pulposus chondrocytic lineage.

Results showing increased production of proteoglycans, e.g., increased ³⁵SO₄ production and/or increased collagen-2 and/or Sox9 protein expression indicate that GDF-6 or the active fragment thereof induce MSCs to differentiate into a nucleus pulposus chondrocytic lineage.

Moreover, results indicating similar levels of expression of transcripts in isolated nucleus pulposus cells and in treated cells indicate that GDF-6 or the active fragment thereof induce MSCs to differentiate into a nucleus pulposus chondrocytic lineage.

MSCs expanded in vitro were encapsulated in alginate beads and NP chondrogenic differentiation was performed using medium with or without BMP-13. Cells were harvested after 7, 14 and 21 days for gene expression and histological analysis. Human NP tissue was used as a positive control.

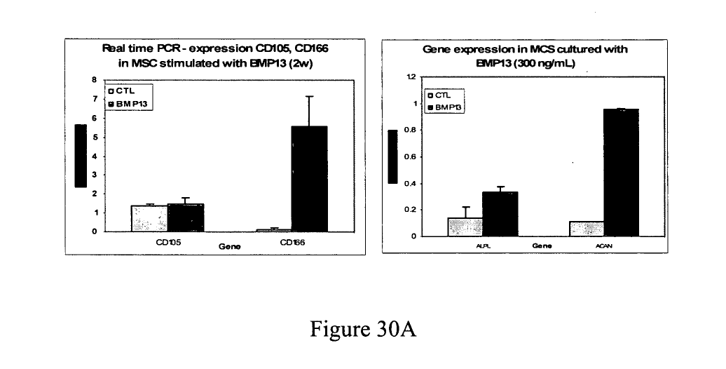

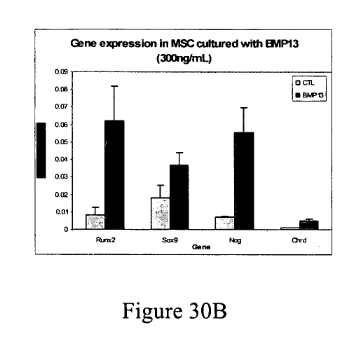

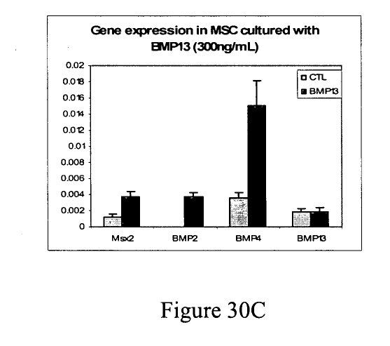

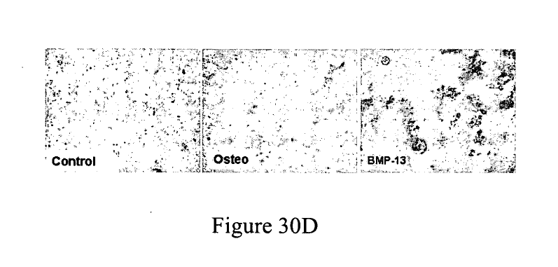

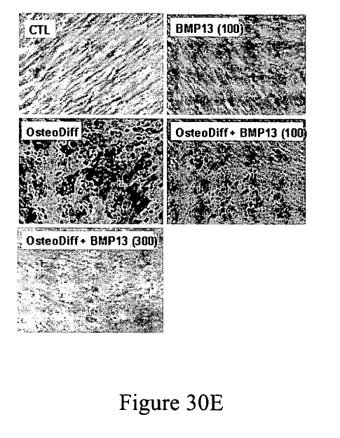

FIGS. 30A-30C show BM MSC cultured in the presence of GDF-6 (300 ng/mL) over a 2 week period and expression of selected genes in the osteogenic, chondrogenic, and adipogenic pathways of differentiation as measured by real-time PCR. The data shows that expression of aggrecan was increased in comparison to Alkaline Phosphatase; and expression of CD166 was increased compared to CD105. Both CD166 and CD105 are markers of stem cell maturation into disc cells. GDF-6 increased expression levels of Sox9, Noggin and Runx2, and to a lesser degree BMP antagonist Chordin. GDF-6 treatment of BM MSC in culture also resulted in increased expression of BMPs 2 and 4 a swell as transcription factor Msx 2.

FIGS. 30D-30E show histochemical analysis of BM MSC cells. FIG. 30D shows Alcian Blue staining of BM MSC cultures treated with control media, osteo-differentiation media , or media with GDF-6 (300 ng/mL), over a two week period and These data demonstrate that GDF-6 stimulated increased proteoglycan production in stem cells, and promotes differentiation into a more disc-like cell. FIG. 30E shows Alizarin red staining of BM MSC cultures treated control media, control media+GDF-6 (BMP13), osteo-differentiation media, or osteo-diff media+GDF-6 (BMP13), over a two week period and varying concentrations of GDF-6. Staining of mesenchymal stem cells with Alizarin Red identifies calcium deposits, an indicator of mineralizing bone tissue. Stem cells cultured in osteo-differentiation media which promotes the formation of bone tissue, demonstrated significant calcium deposition, after 2 weeks in culture. In the presence of GDF-6 alone, these deposits did not form. GDF-6 inhibited the formation of calcium deposits in stem cells cultured in osteo-differentiation media, in a dose dependent manner. These data indicate that GDF-6 inhibits osteodifferentiation of mesenchymal stem cells in a dose dependent manner.

Human intervertebral disc (IVD) degeneration is characterised by a loss of Nucleus Pulposus (NP) tissue structure and function, and by a reduction in disc cell biosynthesis of IVD matrix proteins. The stimulation of pluripotent progenitor cells into mature disc cells producing IVD components provides a therapeutic solution for patients with chronic disc degenerative disease. GDF-6 was studied and as demonstrated in this example, initiates this differentiation in vitro.

### EXAMPLE 19

### Treatment of a Sheep Model of IVD Degeneration with Stem Cells

Sheep are purchased from the University farm, Arthursleigh, Australia, and transported to a veterinary centre a minimum of 2 weeks prior to first experimental procedure and housed in a paddock. Each sheep is premedicated with 0.3 mg/kg diazepam and individually taken to the anesthesia induction area immediately prior to the surgical procedure. The jugular vein is catheterized using a 16 G×3.25 cm catheter after local anesthetic is placed under skin using a syringe and 25 gauge needle. Sheep are anaesthetized with 10 mg/kg ketamine given to effect. The sheep are placed in right lateral recumbency and the right caudal quarter of the sheep clipped and aseptically prepared. A straight incision a few fingerbreadths below the costal margin and parallel to lateral border of the erector spinae muscles is made to allow exposure of the lower lumbar vertebrae (L2 to L6). The approach is made retroperitoneally using electrocautery to divide the subcutaneous tissue, fascia, and thoracolumbar aponeurosis and transversalis fascia in line with the skin incision, the peritoneum is protected and reflected anteriorly by blunt dissection. A retractor is placed between rostral and the iliac crest to aid exposure. The vertebral bodies from L3 to L5 are identified and, with a Deaver retractor, the vessels lying anterior to the spine are protected. Once the appropriate involved vertebra is identified, the psoas muscle is elevated bluntly off the lumbar vertebrae and retracted laterally to the level of the transverse process with a Richardson retractor. Bipolar coagulation of vessels around the vertebrae is also performed. The fibrosus annuli of anterolateral discs of L2 to L5 are identified. Annular fibrosus of two non-contiguous lumbar discs per animal. The incision of fibrous annulus is made to a 6 mm depth using a #9 B-P knife blade. The IVD located between the two punctured IVDs is used as a control. A 27 mm×10 mm titanium screw is implanted into the vertebral body at one level for later identification of levels. One of the punctured levels is treated with stem cells produced according to Example 18, optionally transfected with a nucleic acid encoding full-length GDF-6 or an active domain thereof produced essentially as described in Example 10, and the other punctured disc treated with saline control. In both cases the treatment is injected into the nucleus pulposus of punctured discs. After completion of procedure, the wound is closed in layers.

Radiographs (lateral only) are taken prior to waking of animals. Two weeks after surgery and monthly thereafter, radiography are performed on the assigned sheep using 0.3 mg/kg diazepam and 0.2 mg/kg butorphanol intravenously for sedation. The remaining radiographs and CT scans are performed after euthanasia on the assigned days (3 month, 6 months and 18 months). Disc height is also raidographically determined on a monthly basis (lateral view only) and following euthanasia. IVD height is expressed as a disc height index (DHI). The level of degeneration based on the Thompson grade (1=normal, 4=severely degenerated) is also assessed using MRI.

Following three, six or eighteen months, sheep are euthanized using an overdose of pentobarbitone administered intravenously. After euthanasia the sheep undergo a post-mortem examination and IVDs.

Lumbar vertebral joints are biomechanically tested using an Instron 8874 in 4 modes. Range of motion, constraint to motion, and hysteresis is quantified for the treated joints and compared to controls. Annular tissue samples from treated joint levels and controls are be isolated and tested in tension to determine ultimate strength and tensile modulus.

Disc tissue collected post-mortem is subject to histological analysis to assess the level of disc degeneration. Spines are removed surgically and muscle tissue removed before being submerged in working formalin solution (10% in 0.1M Phosphate buffer). Spines are labeled according to head/tail orientation. After initial fixation, spines are segmented into individual discs labeled +2, +1, −1, −2 in relation to the position of the titanium screw inserted at surgery, and thus identifiable in respect of the treatment administered. Discs are then submerged in de-calcifying solution overnight with agitation to soften bone tissue. Individual discs are sectioned into pieces of tissue no more than 5 mm thick, placed in cassettes for paraffin embedding and thin sectioning. Tissues are then stained with haematoxylin/eosin for tissue architecture analysis.

1. A method for preventing or delaying or treating a spinal disorder and/or spinal pain in a subject, said method comprising administering a modulator of GDF-6 signaling or composition comprising a modulator of GDF-6 signaling to a subject suffering from a spinal disorder and/or spinal pain for a time and under conditions sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject.

2. The method according to claim 1, wherein cells adjacent an end-plate are in a region of ring apophysis.

3. The method according to claim 1, wherein cells adjacent an end-plate are in a region of sub-chondral bone or other bone-comprising surface adjacent an end-plate.

4. The method according to claim 1, wherein the cells in and/or adjacent an end-plate are resting or quiescent in the absence of the administered GDF-6.

5. The method according to claim 1, wherein the cells in and/or adjacent an end-plate are self-renewing in the absence of the administered GDF-6.

6. The method according to claim 1, wherein the cells in and/or adjacent an end-plate are uncommitted in the absence of the administered GDF-6.

7. The method according to claim 1, wherein the cells in and/or adjacent an end-plate are stem cells.

8. The method according to claim 7, wherein the stem cells are resting or quiescent in the absence of the administered GDF-6.

9. The method according to claim 1, wherein the mobilized, activated, or proliferating cells are incorporated into IVD.

10. The method according to claim 1, wherein mobilization, activation, proliferation, enhanced mobilization, enhanced activation or enhanced proliferation of cells in and/or adjacent an end-plate stimulates or enhances chondrogenesis of the cells.

11. The method according to claim 10, wherein the mobilized, activated, or proliferating cells are incorporated into IVD.

12. The method according to claim 1, wherein mobilization, activation, proliferation, enhanced mobilization, enhanced activation or enhanced proliferation of cells in and/or adjacent an end-plate stimulates or enhances proteoglycan production by the cells.

13. The method according to claim 12, wherein the mobilized, activated, or proliferating cells are incorporated into IVD.

14. The method according to claim 1, wherein mobilization, activation, proliferation, enhanced mobilization, enhanced activation or enhanced proliferation of cells in and/or adjacent an end-plate stimulates or enhances collagen production by the cells.

15. The method according to claim 14, wherein the mobilized, activated, or proliferating cells are incorporated into IVD.

16. The method according to claim 1 further comprising monitoring efficacy of therapy.

17. The method according to claim 16 comprising monitoring efficacy of therapy by determining one or more markers associated with chondrogenesis, wherein an increased level of said one or more markers in an IVD is indicative of effective therapy.

18. The method according to claim 17 wherein a marker associated with chondrogenesis is a protein of the chondrogenic pathway selected from the group consisting of: Runx2, Sox9, Noggin, chordin, Msx-1, Msx-2, BMP-2 and BMP-4 and combinations thereof.

19. The method according to claim 18 wherein a marker associated with chondrogenesis is nucleic acid encoding a protein of the chondrogenic pathway selected from the group consisting of: Runx2, Sox9, Noggin, chordin, Msx-1, Msx-2, BMP-2 and BMP-4 and combinations thereof.

20. The method according to claim 16 comprising monitoring efficacy of therapy by determining neovascularization in and/or adjacent the end-plate, wherein absence of neovascularization or absence of enhanced neovascularization an and/or adjacent the end-plate is indicative of effective therapy.

21. The method according to claim 16 comprising monitoring efficacy of therapy by determining mobilization, activation, proliferation or enhanced mobilization, enhanced activation or enhanced proliferation of cells in and/or adjacent the end-plate is indicative of effective therapy.

22. The method according to claim 16 comprising monitoring efficacy of therapy by determining mobilization, activation, proliferation, enhanced mobilization, enhanced activation or enhanced proliferation of cells in and/or adjacent the end-plate, wherein mobilization, activation, proliferation, enhanced mobilization, enhanced activation or enhanced proliferation of cells is indicative of effective therapy.

23. The method according to claim 16 comprising monitoring efficacy of therapy by determining increased expression of one or more proteins regulated by GDF-6 in an IVD, wherein an increased level of said expression is indicative of effective therapy.

24. The method according to claim 23 wherein a protein regulated by GDF-6 is selected from the group consisting of: Runx2, Sox9, Noggin, chordin, Msx-1, Msx-2, BMP-2 and BMP-4 and combinations thereof.

25. The method according to claim 16 comprising monitoring efficacy of therapy by determining increased expression of one or more genes regulated by GDF-6 in an IVD, wherein an increased level of said expression is indicative of effective therapy.

26. The method according to claim 25 wherein a gene regulated by GDF-6 encodes a protein selected from the group consisting of: Runx2, Sox9, Noggin, chordin, Msx-1, Msx-2, BMP-2 and BMP-4 and combinations thereof.

27. The method according to claim 24 comprising determining increased expression of Sox9 in annulus fibrosis cells.

28. The method according to claim 16 comprising monitoring efficacy of therapy by determining proteoglycan in an IVD, wherein an increased level of proteoglycan in an IVD is indicative of effective therapy.

29. The method according to claim 16 comprising monitoring efficacy of therapy by determining collagen I and/or collagen II in an IVD, wherein an increased level of collagen I and/or collagen II in an IVD is indicative of effective therapy.

30. The method according to claim 29, wherein increased collagen I and/or collagen II is in end-plate cells.

31. The method according to claim 1, comprising administering the modulator or composition to a plurality of sites within an IVD and/or a plurality of sites within a nucleus pulposus and/or a plurality of sites adjacent to at least a portion of a nucleus pulposus and/or a plurality of sites within a region of an IVD defined by the internal wall of an annulus fibrosus.

32. The method according to claim 31, wherein the modulator or composition is administered to the plurality of sites in a single administration.

33. The method according to claim 31, wherein the modulator or composition is administered in a patterned manner.

34. The method according to claim 31, comprising administering the modulator or composition to a plurality of sites or in a patterned manner so as to permit said modulator or composition to disperse or distribute evenly throughout the nucleus pulposus.

35. The method according to claim 1, comprising administering the modulator or composition via a medical device comprising a delivery conduit having a proximal end attachable to a source of the modulator of GDF-6 signaling or the composition and an emitter structure at a distal end of the delivery conduit, wherein the emitter structure defines a plurality of spaced discharge apertures through which the modulator or composition is delivered.

36. The method according to claim 1, comprising administering the modulator or composition by injection through one or more sites in bone and in sufficient proximity to an end-plate in or adjacent an IVD in need of treatment such that the modulator or composition is capable of mobilizing, activating, proliferating, enhancing mobilization, enhancing activation or enhancing proliferation of cells in and/or adjacent the end-plate of the IVD in need of treatment.

37. The method according to claim 36, comprising administering the modulator or composition by injection to a single site below the end-plate in or adjacent an IVD in need of treatment.

38. The method according to claim 36, comprising administering the modulator or composition to a plurality of sites.

39. The method according to claim 38, comprising administering the modulator or composition by injection through a plurality of sites in bone wherein each of said sites is in sufficient proximity to an end-plate in or adjacent an IVD in need of treatment such that the modulator or composition is capable of mobilizing, activating, proliferating, enhancing mobilization, enhancing activation or enhancing proliferation of cells in and/or adjacent an end-plate of each IVD in need of treatment.

40. The method according to claim 38, comprising administering the modulator or composition by injection through one or a plurality of sites in bone using a medical device comprising a delivery conduit having a proximal end attachable to a source of the modulator or composition and an emitter structure at a distal end of the delivery conduit, wherein the emitter structure defines a plurality of spaced discharge apertures through which the modulator or composition is delivered, such that the number of injection sites in bone is less than the number of IVDs in need of treatment.

41. The method according to claim 40, comprising administering the modulator or composition by injection through a single site in bone and dispering the modulator or composition to a plurality of IVDs in need of treatment.

42. A method for preventing or delaying or treating a spinal disorder and/or spinal pain in a subject, said method comprising administering a modulator of GDF-6 signaling or composition comprising a modulator of GDF-6 signaling to a subject suffering from a spinal disorder and/or spinal pain for a time and under conditions sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject, wherein said administration comprises:
(i) accessing a region of an IVD by surgical intervention or injection;
(ii) providing or obtaining a medical device comprising the modulator or composition wherein the medical device comprises a delivery conduit having a proximal end attachable to a source of the modulator of GDF-6 signaling or the composition and an emitter structure at a distal end of the delivery conduit, wherein the emitter structure defines a plurality of spaced discharge apertures through which the modulator or composition is delivered;
(iii) inserting the emitter structure of the medical device at least partially into the accessed region of the IVD;
(iv) manipulating the emitter structure so that the emitter structure is positioned within the IVD and/or at least partially surrounds or is positioned within the nucleus pulposus and/or a region of the IVD defined by an internal wall of the annulus fibrosus; and
(v) discharging the modulator or composition through the apertures of the device so as to administer said modulator or composition to a plurality of sites within the IVD in a single administration and/or at least partially surrounds or is positioned within the nucleus pulposus and/or a region of the IVD defined by an internal wall of the annulus fibrosus, thereby administering the modulator or composition to the subject.

43. A method for preventing or delaying or treating a spinal disorder and/or spinal pain in a subject, said method comprising administering a modulator of GDF-6 signaling or composition comprising a modulator of GDF-6 signaling to a subject suffering from a spinal disorder and/or spinal pain for a time and under conditions sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject, wherein said administration comprises providing or obtaining an agent delivery system that comprises:
(i) a dispenser defining a reservoir and an outlet port in communication with the reservoir;
(ii) a high density, immiscible, non-reactive, biocompatible displacement fluid comprising the modulator or composition, said fluid being contained within the reservoir ; and
(iii) a displacement device arranged in the reservoir for displacing the fluid through the outlet port of the dispenser.

44. The method according to claim 43, wherein the agent delivery system comprises a receptacle for the fluid, the receptacle having a mounting formation for mounting the receptacle to the dispenser so that an interior of the receptacle is in communication with the outlet port of the dispenser.

45. The method according to claim 44, wherein the receptacle comprises a cannula with at least one discharge opening.

46. The method according to claim 45, wherein the cannula is elongate having a side wall defining a plurality of axially spaced discharge openings.

47. The method according to claim 46, wherein each discharge opening includes an occluding device for inhibiting back flow of the fluid into the interior of the cannula.

48. The method according to claim 46, wherein each of at least some of the openings open out into a recessed region of the side wall of the cannula.

49. The method according to claim 45, wherein the cannula is shaped and dimensioned to access a plurality of sites simultaneously.

50. The method according to claim 45, wherein the cannula is flexible to be able to be directed to a desired location in a patient's body.

51. The method according to claim 44, wherein the agent delivery system comprises a reaming tool for forming a passage through bone at a site in the patient's body into which the receptacle is to be inserted.

52. The method according to claim 51, wherein the reaming tool is steerable.

53. A method for preventing or delaying or treating a spinal disorder and/or spinal pain in a subject, said method comprising administering a modulator of GDF-6 signaling or composition comprising a modulator of GDF-6 signaling to a subject suffering from a spinal disorder and/or spinal pain for a time and under conditions sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject, wherein said administration comprises providing or obtaining an agent delivery system that comprises:
(i) an elongate body defining a lumen;
(ii) at least one opening defined in the body through which the modulator or composition can be discharged; and
(iii) an occluding device contained in a receptacle in register with at least one of said openings, said occluding device being for closing off the opening(s) to thereby inhibit back flow of the modulator or composition into the lumen of the body after being discharged through the opening(s).

54. The method according to claim 53, wherein the body has a mounting formation for mounting to a dispenser so that an interior of the body is in communication with an outlet port of the dispenser.

55. The method according to claim 54, wherein the body comprises a cannula having a side wall defining a plurality of axially spaced discharge openings.

56. The method according to claim 55, wherein a proportion of said plurality of openings open out into a recessed region of the side wall of the cannula.

57. The method according to claim 55, wherein the cannula is shaped and dimensioned to access a plurality of sites simultaneously.

58. The method according to claim 55, wherein the cannula is flexible to be able to be directed to a desired location in a patient's body.

59. A method for preventing or delaying or treating a spinal disorder and/or spinal pain in a subject, said method comprising administering a modulator of GDF-6 signaling or composition comprising a modulator of GDF-6 signaling to a subject suffering from a spinal disorder and/or spinal pain for a time and under conditions sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject, wherein said administration comprises providing or obtaining a reaming tool for forming a passage in bone in a patient's body, the reaming tool comprising:
(i) a reaming head;
(ii) a pivot to which the reaming head is pivotally mounted; and
(iii) a steering mechanism for steering the reaming head through body tissue and bone.

60. The method according to claim 59, wherein the reaming head is omni-directionally pivotally mounted relative to the pivot.

61. A method for preventing or delaying or treating a spinal disorder and/or spinal pain in a subject, said method comprising administering a modulator of GDF-6 signaling or composition comprising a modulator of GDF-6 signaling to a subject suffering from a spinal disorder and/or spinal pain for a time and under conditions sufficient to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject, wherein said administration comprises:
(i) inserting a cannula comprising the modulator or composition into a site in the vertebral column of the subject, wherein the cannula is mounted on a dispensing device; and
(ii) using a high density, immiscible, non-reactive, biocompatible displacement fluid contained within a reservoir of the dispensing device to discharge the modulator or composition from the cannula.

62. The method according to claim 61, comprising inserting the cannula into the patient's body percutaneously to thereby access the site of insertion into the vertebral column of the subject.

63. The method according to claim 61 comprising forming a passage through tissue and bone.

64. The method according to claim 61 comprising forming a passage through one or more vertebrae on at least one side of an IVD to be treated and delivering the modulator or composition such that it is capable of mobilizing, activating, proliferating, enhancing mobilization, enhancing activation or enhancing proliferation of cells in and/or adjacent an end-plate.

65. The method according to claim 64, comprising delivering the the modulator or composition by injection through a number of vertebrae simultaneously.

66. The method according to claim 61, comprising inserting the cannula into the patient's body trans-sacrally.

67. The method according to claim 61, comprising inserting the cannula into the patient's body peri-annularly adjacent an IVD in need of treatment.

68. The method according to claim 67, wherein peri-annular insertion of the cannula comprises a mode of insertion selected from the group consisting of: trans-sacral epidural insertion, transforaminal epidural insertion and interlaminar periannular insertion.

69. The method according to claim 67, wherein peri-annular insertion of the cannula comprises negotiating the cannula through the epidural space from one side to a contralateral side within the spinal canal close to an annulus of an IVD and negotiating the cannula in extra-canal space in the periannular area.

70. The method according to claim 61 comprising manipulating the cannula about cartilaginous tissue in the patient's body.

71. The method according to claim 1, wherein the modulator of GDF-6 signaling modulates the activity and/or expression of a molecule selected from the group consisting of GDF-6, MSX-1, MSX-2, BMPR-1A, BMPR-IB, BMPR-II, Smad-1, Smad-5, Smad-8, Smad-4 and mixtures thereof.

72. The method according to claim 1, wherein the modulator of GDF-6 signaling is a peptide or polypeptide.

73. The method according to claim 72, wherein the peptide or polypeptide comprises GDF-6 or an active fragment thereof or an analog thereof or a derivative thereof.

74. The method according to claim 72, wherein the peptide or polypeptide comprises MSX-1 or an active fragment thereof or an analog thereof or a derivative thereof.

75. The method according to claim 72, wherein the peptide or polypeptide comprises MSX-2 or an active fragment thereof or an analog thereof or a derivative thereof.

76. The method according to claim 1, comprising administering a cell comprising and/or expressing the modulator of GDF-6 signaling.

77. The method according to claim 76, wherein the cell is a stem cell.

78.-85. (canceled)

86. A composition for modulating GDF-6 signaling in an intervertebral disc or a cell or tissue thereof sufficient to reduce, delay or prevent intervertebral disc degeneration in a subject and/or to induce and/or enhance intervertebral disc regeneration in a subject, said composition comprising (i) an amount of a modulator of GDF-6 signaling sufficient to to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject; (ii) a suitable carrier or excipient; and (iii) instructions for administering the composition to an intevertebral disc of a subject.

87. The composition according to claim 86, wherein said composition comprises a stem cell comprising or expressing a modulator of GDF-6 signaling.

88. The composition according to claim 86, wherein said composition comprises an amount of a polypeptide modulator of GDF-6 signaling.

89. The composition according to claim 86, wherein the composition is a slow release composition.

90. The composition according to claim 86, wherein the composition has a viscosity that permits it to disperse or distribute evenly throughout the nucleus pulposus of a subject.

91. A method for producing a composition for modulating GDF-6 signaling in an intervertebral disc or a cell or tissue thereof to thereby reduce, delay or prevent intervertebral disc degeneration in a subject and/or to induce and/or enhance intervertebral disc regeneration in a subject, said method comprising mixing or otherwise combining an amount of a modulator of GDF-6 signaling sufficient to to mobilize, activate or proliferate cells in and/or adjacent an end-plate or enhance mobilization, activation or proliferation of said cells to thereby reduce, delay or prevent intervertebral disc (IVD) degeneration in the subject and/or to induce and/or enhance IVD regeneration in the subject and a suitable carrier or excipient and optionally, providing instructions for administering the combination to an intevertebral disc of a subject.

92. The method of claim 91, wherein the carrier or excipient has a viscosity that permits the composition to disperse or distribute evenly throughout the nucleus pulposus of a subject.
